(19) United States
(12) Patent Application Publication
(10) Pub. No.: US 2009/0029370 A1
(43) Pub. Date: Jan. 29, 2009

(54) REAL TIME NUCLEIC ACID DETECTION IN VIVO USING PROTEIN COMPLEMENTATION

(75) Inventors: Natalia E. Broude, Natick, MA (US); Charles R. Cantor, Del Mar, CA (US); Maria A. Burton, Waltham, MA (US); Vadim V. Demidov, Boston, MA (US)

Correspondence Address: RONALD I. EISENSTEIN, 100 SUMMER STREET, NIXON PEABODY LLP, BOSTON, MA, 02110, US

(73) Assignee: TRUSTEES OF BOSTON UNIVERSITY, Boston, MA (US)

(21) Appl. No.: 12/091,682

(22) PCT Filed: Oct. 27, 2006
(86) PCT No.: PCT/US2006/042210

§ 371 Date: Apr. 25, 2008

Related U.S. Application Data
(60) Provisional application No. 60/730,746, filed on Oct. 27, 2005.

(51) Int. Cl. C12Q 1/68; C07H 21/04; C12N 5/10; C12N 1/19; C12N 1/21

(52) U.S. Cl. 435/6; 536/23.1; 435/354; 435/325; 435/348; 435/254.2; 435/252.3

(57) ABSTRACT

The present invention relates to a method to detect nucleic acid molecules, such as RNA molecules in vivo using real time protein complementation methods. The invention further relates to methods for detecting nucleic acids, for example RNA in real-time in living cells with a high sensitivity, using a novel split biomolecular conjugate of the invention.

### CROSS REFERENCE TO RELATED APPLICATIONS

This application claims the benefit under 35 U.S.C. 119(e) of U.S. Provisional Patent Application Ser. No. 60/730,746, filed on Oct. 27, 2005, the contents of which are herein incorporated by reference in their entirety.

### FIELD

The present invention is directed to compositions and methods for the in vivo detection of nucleic acids. More preferably, the compositions and methods allow for the sensitive and real time detection of RNA in vivo.

### BACKGROUND

RNA is an active participant in a multi-step process broadly determined as gene expression, which includes transcription and processing of RNA within nucleus, export from the nucleus, transport through cytoplasm and translation within ribosomes. Additionally, non-coding RNAs, an ever growing class of RNA molecules, participate in a variety of post-transcriptional and post-translational events concerning all cellular macromolecules, proteins, DNA and RNA: RNA editing, RNA modifications, DNA methylation and protein modifications (Kiss, 2002, Mattick, 2004; Huttenhofer et al., 2005). To perform these multiple functions, RNA must be in the correct cellular location at the correct time. In other words, spatial and temporal localization of RNA molecules within the cell has recently emerged as an important mechanism of cellular biology.

It has been realized that localization of RNA not only regulates protein synthesis, but also creates gradient of morphogens and determines formation of cell lineages and cell organelles (for review, see Kloc et al., 2002). If RNA is not functioning properly, it may result in various forms of pathology. The dynamic and unstable nature of RNA and its important role in multiple functions linked to its movements and localization, has created both an interest and challenge.

One example of the difficulties currently faced in the field of RNA localization in vivo is the lack of adequate methods to estimate the diffusion coefficient of poly(A)+-mRNA within the nucleus. Values have been reported that differ by two orders of magnitude, namely, 0.03-0.6 μ²/sec and 9 μ²/sec (Politz et al., 1998, Politz et al., 1999, Molenaar et al., 2004). Thus, it is evident that to better understand functions and behavior of various RNAs in vivo, new methods of studying their localization and movements within living cells are urgently needed.

In order to visualize RNA within the cell, it should be selectively labeled. Different strategies for in vivo RNA labeling have been suggested and used (for a review, see Pederson, 2001). Most of them use different RNA-specific hybridization probes and microinjection or lipofection for probe delivery to the nucleus or cytoplasm. Another possibility is to label RNA beforehand and inject it into the cell. Self-ligating quenching probes were successfully used by Kool and co-workers for RNA hybridization within bacterial cells (Sando & Kool, 2002). Fluorescent 2′-O-methyl-RNA probes have been shown to be more stable than unmethylated oligonucleotides and were used by several groups (Carmo-Fonseca et al, 1999; Molenaar et al., 2001; Molenaar et al., 2004). Molecular beacons and caged-fluorescein labeled antisense oligonucleotides present advanced variants of RNA hybridizing probes (for reviews see Politz, 1999, Pederson, 2001). The advantage of molecular beacons and caged probes is that they do not produce signal unless they hybridize to the target or are illuminated, respectively; this allows lower background and consequently better sensitivity (Sokol et al., 1998; Perlette & Tan, 2001; Matsuo, 1998; Tsuji et al, 2001; Sei-Iida et al., 2000).

The most severe limitation of this hybridization strategy is the low sensitivity of hybridization due to the low concentration of RNA within the cell. In most cases only highly abundant RNA species can be detected (β-actin mRNA, c-fos mRNA, basic fibroblast growth factor RNA, or total poly (A)-RNA). Another obstacle of using oligonucleotide probes for RNA detection in vivo is their fast accumulation in the nucleus (Tsuji et al., 2000; Molenaar et al., 2001).

Another strategy to study RNA in vivo uses fusions between RNA-binding proteins and fluorescent proteins and introduction of the protein-binding tags within RNA-target. Two groups used systems based on interaction of the MS2 coat protein and corresponding RNA motif (Bertrand et al, 1998; Beach et al., 1999; Beach & Bloom, 2001) and another group used a system based on U1A splicing protein (Takizawa & Vale, 2000). It was hoped that this approach would allow for monitoring real-time movements of specific RNAs in yeast cells (Bertrand et al, 1998; Beach et al., 1999; Corral-Debrinski et al., 2000; Beach & Bloom, 2001), and neurons (Rook et al., 2000). This technique represents a first attempt to marry fluorescent proteins with RNA studies within the cells. However, a substantial limitation is the high background signal of the fully functional fluorescent protein fused to the RNA-binding protein making actual monitoring difficult.

Further, analysis of RNA in vivo mostly relies on fluorescent detection methods, where RNA-specific fluorescent probes are either delivered to (e.g., molecular beacons) or are synthesized inside the cell (e.g., the enhanced green fluorescent protein, EGFP, fused to an RNA-binding protein, e.g. MS2 coat protein) [see (14,15) for reviews]. However, these methods have severe drawbacks and limitations. Pre-labeled oligonucleotide detector probes must be modified to be nuclease-resistant and must be delivered into the cell using invasive techniques (16). The second approach using RNA-binding proteins involves modifying the RNA of interest with multiple copies (up to 96) of the RNA-binding protein recognition sequence in tandem, while the RNA-binding protein is fused to a full-size fluorescent protein. As a result, a large multimeric complex of fusion proteins gets assembled on the RNA of interest (17-20), which may impair normal movement and migration of RNA in the cell. In bacteria there is also a high background fluorescence caused by the full-length fluorescent protein, which tends to aggregate and the aggregates can be confused with the RNA-protein complexes. In eukaryotes, separation of the fluorescent protein and RNP complexes in different compartments helps in some cases (21), but generally high fluorescent background attributed to the full-length fluorescent protein limits sensitivity of this approach. Thus, all current methods of RNA labeling are suffering from the high non-specific background and lack of signal amplification; therefore most of them are limited by detection of abundant RNA species. In addition, there is a great need to detect RNA in real time.

### SUMMARY OF THE INVENTION

The inventors of the present invention discovered a method to detect nucleic acid molecules, such as RNA molecules in vivo using real time protein complementation methods. In particular, the invention provides methods for detecting nucleic acids, for example RNA, in vivo with a high signal:background ratio, thus enabling detection of RNA with high sensitivity. Further, the methods of the invention provide methods and components for the detection of DNA and RNA within a live cell using non-invasive methods. More specifically, the compositions of the present invention comprise a detector molecule which comprises a detector protein that is split into two or more polypeptide fragments which are attached to nucleic acid binding motifs, which may function independently or cooperate to bind a single site. Re-association of the detector polypeptide protein fragments into a functional active detector protein will only occur in the presence of a target nucleic acid, such as RNA or DNA, which as been modified to comprise a reporter nucleic acid binding sequence (for example an aptamer), allowing the interaction of the nucleic acid-binding motifs with their cognate binding site(s) on the reporter nucleic acid sequence. This interaction brings together the fragments of the detector protein, allowing for immediate signal detection. The polypeptides of the detector molecule, in particular the detector protein components, are in the active configuration but are not active alone, but on reconstitution they immediately form the active protein, therefore the production of signal occurs in real time in the presence of target nucleic acid. In one embodiment, the target nucleic acid is RNA. Also, one embodiment encompasses a RNA-binding protein or peptide as the nucleic acid binding motif component of the detector molecule.

As described herein, a “detector construct” refers to the nucleic acid sequence encoding a detector molecule comprising the above described detector protein (either fluorescent or enzymatic) that is split into two or more activated polypeptide fragments, each fragment conjugated with an nucleic acid-binding motif. The polypeptide fragments, when brought together by binding of the conjugated nucleic acid binding motif to a target nucleic acid, reconstitute the fully active protein, and can be immediately detected.

As described herein, a “reporter construct” refers to the nucleic acid sequence encoding a reporter molecule, comprising above described nucleic acid for example a RNA or DNA encoding a gene of interest, and a target nucleic acid binding sequence, for example an aptamer. The nucleic acid binding sequence is recognizable by the nucleic acid-binding motif of the detector construct. The nucleic acid binding sequence may be a nucleic acid, protein, aptamer, or aptamer tag.

In one embodiment, the detector protein is a fluorescent molecule, such as, for example EGFP. In one embodiment of the present invention, the fluorescent reporter is EGFP that is split into an alpha fragment (approximately amino acids 1-158) and a beta fragment (approximately amino acids 159-239). The alpha fragment contains mature fully formed chromophore, which does not fluoresce alone, but is primed to fluoresce and when paired with the beta fragment, immediately fluoresces. The immediacy of the fluorescence allows for the real-time detection of RNA in vivo which is currently unavailable in the art. Importantly, the alpha and the beta fragments do not reassociate or fluoresce in the absence of target nucleic acid, for example target RNA.

In another embodiment, the fluorescent protein is green fluorescent protein (GFP) or enhanced green fluorescent protein (EGFP). In alternative embodiments, the fluorescent protein is yellow fluorescent protein (YFP), an enhanced yellow fluorescent protein (EYFP), a blue fluorescent protein (BFP), an enhanced blue fluorescent protein (EBFP), a cyan fluorescent protein (CFP), an enhanced cyan fluorescent protein (ECFP) or a red fluorescent protein (dsRED) or any other natural or genetically engineered fluorescent protein of those listed above. In yet further embodiments, the reconstituted fluorescent proteins may comprise of a mixture of fragments from the same or a combination any of the above listed fluorescent proteins.

Alternatively, the detector protein is an enzyme that allows for signal amplification. The enzyme may be, for example, beta-galactosidase, beta-lactamase, beta-glucosidase, beta-glucuronidase, chloramphenicol acetyl transferase. In specific embodiments the detector enzyme is beta-lactamase.

In an important embodiment of the present invention, the compositions and methods provide a reporter assay which allows for the real time detection of RNA in vivo. In one embodiment, cells of interest are created to stably express a detector construct. The detector construct expresses a detector molecule which does not produce a signal from the detector protein in the absence of target RNA and thus does not fluoresce or show activity. The cells can also express a reporter construct encoding a reporter molecule comprising a binding site for the detector molecule's nucleic acid binding motif(s) and a gene of interest. The expression of such a reporter construct can be induced. When such a reporter molecule is expressed within a cell, the detector molecule will bind the target RNA and facilitate detector protein reconstitution and production of an active protein and signal. This allows for the real time detection of RNA expression in vivo and is exemplified in FIGS. 10-11, and 18-19 and Examples 9 to 11.

In an alternative embodiment, the cells of interest can express a bicistronic nucleic acid sequence comprising the reporter molecule where the detector construct is constitutively expressed and the expression of the reporter construct is operatively linked to a promoter of interest. In a related embodiment, the reporter construct (operatively linked to a promoter of interest) and detector construct are on separate nucleic acid sequences. When the promoter is activated within the cell by certain stimuli, the binding site(s) for the target nucleic acid sequence becomes available and binding of the nucleic acid binding protein of the detector molecule facilitates the association of the detector polypeptide fragments and formation of the active detector protein which can be immediately detected. This embodiment allows for promoter function to be studied in real time in vivo. For example, the regulation of a gene can be studied in real time by transfecting a plasmid expressing a promoter driving an RNA binding site and analyzing any baseline detector construct activity. In a related embodiment, the cells are contacted with i) a compound or compounds; or ii) subjected to a variety of conditions that may alter the activity of the promoter, thereby increasing, decreasing or causing no change in the transcription of the RNA. This alteration is immediately detected by the detector construct.

In a similar embodiment, a “tet-on” or “tet-off” system may be utilized in the methods of the present invention. For example, cells of interest may be created to stably express the detector construct of the present invention. In addition, the cells may also stably express a tet-on or tet-off regulatable reporter construct enabling controlled transcription of the target binding site(s) and gene of interest for the detector molecules nucleic acid binding motifs). For example, in a tet-off system, the cells would immediately express the gene of interest and the reporter construct would be active and immediately detectable. Upon the addition of tet, the transcription of the reporter construct (comprising the gene of interest and target nucleic acid binding site) is shut off and only the transcripts that have been already transcribed can be analyzed in real time. In this tet-off example one could analyze the stability of RNA, such as for example, its half-life, in vivo in real time by detecting the diminishing reporter fluorescence or activity.

In an alternative embodiment, the use of a “tet-on” construct allows for the stable expression of a gene of interest in a living cell, whereby the addition of tet to the system turns on transcription of the reporter construct. The detector molecule of the present invention is then able to bind its target RNA and is immediately detectable. In this embodiment, RNA localization may be studied in real time as dictated by the person conducting the experiment.

In yet another embodiment of the present invention, the reporter assay is in vivo in an organism. For example but not limited to, animals, non-human animals, mouse, zebrafish, C. elegans, or frog may be created to express both a detector construct and a gene of interest with an RNA binding site that will bind to the detector construct. The expression of both constructs within the organism allows for a transcript of interest to be analyzed throughout development by talking samples from Day 1 embryo throughout adulthood. In one embodiment, the organism is a mammal. In one embodiment the mammal is a mouse, and in another embodiment the organism is a transgenic organism, for example but not limited to a transgenic mouse.

Alternatively, the compositions and methods of the present invention provide for the real time detection of RNA in vitro similar to in situ hybridization. In such an embodiment, a cell expressing a reporter construct comprising a RNA of interest with a RNA binding sequence that will bind to the nucleic acid binding motif of the detector molecule is permeablized, and the detector constructs of the invention are administered to the cell. If the RNA of interest is expressed, the detector construct will bind to the RNA binding sequences and the localization and abundance of RNA can be determined. The advantages of this system over traditional in situ hybridization techniques are the ability to detect low abundance RNAs (due to the signal amplification attainable by a detector construct which utilizes an enzyme reporter) and the ability to finish the assay quickly and without radioactivity. The immediacy of the detector construct detection is highly desirable. Currently, traditional methods of performing in situ hybridization (e.g. with radioactivity) can take weeks to months before even knowing if the experiment worked or not. In addition, the RNA probes used in traditional in situ hybridization are difficult to prepare due to the ease of RNA degradation and the use of radioactivity. In the methods of the present invention there is no need for radioactivity or RNA probes. The detector constructs are easily prepared, for example, from bacteria, as is known to those of skill in the art. Such an assay is similar to immunohisto- or immunocyto-chemistry techniques whereby antibodies are used to detect protein in vitro. Here, RNA is detected.

In an alternative embodiment, the compositions and methods of the present invention provide for the real time detection of nucleic acid, in particular RNA and DNA in vivo, which relates to use of the invention when both the RNA and protein constructs are manipulated in cells out side the body, for example within a test tube, as a non-limiting example, detection of RNA in cells that are ex vivo.

Furthermore, the compositions and methods of the present invention may be used in embodiments to detect DNA in vivo in real time. For example, the detector construct of the present invention may comprise a detector protein (either fluorescent or enzymatic) that is split into at least two inactivated polypeptide fragments, each fragment associated with a nucleic acid-binding motif. The polypeptide fragments, when brought together by the presence of target nucleic acid, for example the aptamer, form a fully active detector protein, and can be immediately detected. For example, the methods of the present invention allow for the real time detection of replication of various loci within the genome. The nucleic acid-binding motifs associated with the detector proteins in the detector construct are specific for various regions of the RNA or DNA and the replication of those loci can be detected in real time in vivo.

Similarly, one could detect chromosomes in real time in vivo using the methods and compositions of the present invention. For example, the detector construct may comprise a detector protein associated with telomerase binding proteins and expressed within cells. In this way, teleomeres can be detected in real time over the lifespan of the cell. The advantage of the compositions and methods presented herein is that the specificity is increased by providing a telomerase binding protein that is dissected into two halves, each half associated with one half of the detector protein. The coordinated binding of the two halves of the telomerase binding proteins to a single binding site ensures that there is little to no detector activity prior to target recognition.

In another embodiment, the present invention provides kits suitable for the method of the invention, in particular detecting the RNA in vivo. In one embodiment, the kits comprise the detector construct and the inducible reporter construct. In one embodiment the reporter construct and detector construct are on separate nucleic acid molecules. In another embodiment, they are on both part of bicistronic nucleic acid molecule. In another embodiment, the reporter molecule can be modified to add the practitioner's gene of interest, and in another embodiment, the expression of the reporter molecule is operatively linked to a tet-on or tet-off promoter. In another variant embodiment, the reporter construct, comprising the target nucleic acid sequence can be modified by the practitioner to be regulated by the practitioner's promoter of interest. In each of these embodiments, the kits comprise a choice of split polypeptides for the detector protein in the detector molecule, and also comprise instructions and reagents for detecting a signal from the detector protein. In some embodiments, the kits also comprise reagents suitable for capturing and/or detecting the present or amount of target nucleic acid in a sample. The reagents for detecting the present and/or amount of target nucleic acid can include enzymatic activity reagents or an antibody specific for the assembled protein. The antibody can be labeled.

The combination of protein complementation and an enzymatic reporter allows for a substantially reduced background and signal amplification. This results in an in vivo RNA detection technique capable of analyzing nucleic acids of moderate abundance.

In a further embodiment, the invention provides for kits that comprise means for detecting RNA in vivo.

Other aspects of the invention are disclosed infra.

### BRIEF DESCRIPTION OF THE DRAWINGS

FIG. 1: Transcriptional system to study the effect of the aptamer on gene expression in Saccharomyces cervisiae (Blake et al., 2003).

FIG. 2: Insertion of the aptamer at the 3′ end of the marker gene does not affect gene expression in yeast. An aptamer tag was inserted at the 3′ end of EGFP (see FIG. 1 for plasmids description). Cultures were grown to a logarithmic phase in the presence of 0.2% galactose and 40 ng/ml ATc. Numbers 1-4 correspond to four different chromosomal integrations of the inducible construct. 50,000 cells were assayed by FACS in each culture.

FIG. 3: Outline of protein fusions containing fragments of EGFP and eIF4A used in this study. Eukaryotic initiation factor was split into two domains, and each domain was fused to the two fragments of EGFP to generate EGFP(A)-eIF4A(F1) and EGFP(B)-eIF4A(F1). Numbers indicate the amino acid range included in each fragment. (N,N-terminus; C, C-terminus).

FIG. 4: Expression of two chimeric proteins in bacterial cells in the absence of aptamer-tag does not reconstitute fluorescence. EGFP(A)-eIF4A(F1) and EGFP(B)-eIF4A(F1) were co-expressed in BL21 (DE3) cells. Cultures were induced with 1 mM IPTG for 3 hours at 37° C. and complex formation was monitored by fluorescence flow cytometry. BL21 (DE3) cells expressing full-length EGFP were used as a positive control for induction (bEGFP), and expression of A-F1 and B-F1 was considered as a negative control.

FIG. 5: Schematics of protein complementation in vivo with two adjacent aptamer-tags and two peptides independently interacting with cognate aptamer. The split marker protein is in grey, and the RNA-binding peptides (probe protein) are in orange. (FIG. 5B) Three aptamers found by in vitro selection against CQ peptide of HIV Tat protein (SEQ ID NO.2); HIV rev peptide (SEQ ID NO.1) and HTLV Rex peptide (SEQ ID NO.3), respectively (see Table 1 for Kd and refs). These or similar structures can be used in our project as RNA tags in an alternative design (as opposed to split-protein approach).

FIG. 6: Outline of the protein complementation assay supported by nucleic acids interactions for RNA studies in vivo. A protein with enzymatic activity or fluorogenic properties is split into two inactive parts (termed alpha- and beta-subunits). These parts are expressed in vivo as chimeras with another protein, which has an RNA-binding domain. An ideal RNA-binding protein would consist of two parts, which are inactive by itself but are active together. In the presence of an RNA, which contains a motif recognizable by the RNA-binding protein, the activity of the marker protein is restored. In case the protein is an enzyme, the signal is amplified. In case the protein is fluorogenic, there is no signal amplification, but there is also no background, because fluorescence is completely dependent on the presence of target RNA.

FIG. 7: Schematics of RNA detection in vivo. Target RNA is synthesized in vivo or injected into the cell. Two protein chimeras are synthesized in vivo or transfected into a cell. Each chimera contains a part of a marker protein and a domain, which specifically binds special secondary structure (or motif) within the target RNA. In the presence of target RNA which carries a tag, specifically recognized by the RNA-binding protein, a protein marker is assembled, which is detected by its activity (fluorescence or enzymatic activity).

FIG. 8: Schematic of one embodiment of the invention whereby RNA expression is analyzed in vivo in real time. A gene of interest and a RNA binding site is encoded on a plasmid and transfected into cells that have the reporter construct of the present invention stably integrated. When the gene (and RNA binding site) is expressed, the reporter construct is activated.

FIG. 9: Protein complementation of split EGFP detects RNA in live bacterial cells. (a) Expression of two protein fusions each containing fragment of a split eIF4A and a split EGFP does not result in a fluorescent signal. (b) Expression of full-length EGFP results in uniformly fluorescent E. coli cells. (c) Co-expression of two protein fusions and the RNA transcript with aptamer results in a fluorescent signal often localized to the cell poles. Top row: Molecular constructs expressed in E. coli. Second row: Plasmids expressing components of the complementation complex. Third row: Fluorescence distributions of cells expressing EGFP complementing complexes obtained by FACS; black, before IPTG induction; red, after IPTG induction. Bottom row: Fluorescent micrograph of E. coli cells expressing corresponding components of the complementing complexes.

FIG. 10: Dynamics of transcription within a single bacterium. (a) Time-lapse fluorescent micrographs of E. coli cells expressing RNA aptamer-eIF4A complementing complex at 30 min intervals. Phase-contrast micrographs did not change during the experiment.

FIG. 11: The kinetics of fluorescence facilitated by RNA detection in living cells. (b) Kinetics of fluorescence changes in different single cells (c) Kinetics of total fluorescence changes of all cells in the field. (d) Changes in fluorescence distributions in real-time in a single bacterial cell measured along the long axis of the cell.

FIG. 12: Cells expressing RNA without aptamer sequence do not display high fluorescence Fluorescence distributions of the cells expressing protein components of the complementation complex in the presence of the lacZ transcript obtained by FACS. Black, before IPTG induction; red, after IPTG induction. For comparison, fluorescence distribution of the cells expressing RNA containing aptamer sequence and complementing proteins is shown in blue.

FIG. 13: Cells expressing aptamer-containing target RNA from a higher copy vector (˜100 copies) display higher fluorescence. (a) Schematics of plasmids expressing protein fusions and RNA transcript containing aptamer sequence. (b) Fluorescence distributions of the cells expressing EGFP complementing complexes obtained by FACS. Black, before IPTG induction; red, after IPTG induction. For comparison, fluorescence distribution of the cells with aptamer expressed from the lower copy number (˜40) plasmid is shown in blue.

FIG. 14: Fluorescence spectra of the cells expressing full-length EGFP (a) and of the reconstructed nucleoprotein complex containing split EGFP (b) are different. The cell suspensions were diluted to an optical density of 0.5 (OD₆₀₀=0.5) and fluorescence was recorded using F-2500 fluorimeter (Hitachi). Non-induced cells with the same density were used as controls for scattering.

FIG. 15: Newly divided cells are not fluorescent. (a) Phase-contrast images taken at the indicated time points. (b) Corresponding fluorescent pictures.

FIG. 16: Design of fluorescent protein complementation based on binary peptide-RNA aptamer interactions. Two fragments of EGFP, α and β-, and fused with two viral peptides, HIV-1 Rex peptide and bacteriophage λN peptide. In the presence of an RNA transcript bearing two corresponding aptamers, the two peptides interact with cognate aptamer and bring together two fragments of split EGFP. Re-assembly of EGFP results in appearance of intense fluorescence.

FIG. 17: Protein complementation of split EGFP based on binary aptamer/peptide interaction detects RNA in live bacterial cells. Low concentration of IPTG (0.01 mM) allows discrimination of signal from background. A, Fluorescence distributions of cells induced by 1.00 mM IPTG. B, Fluorescence distributions of the cells induced by decreasing concentrations of IPTG.

FIG. 18: E. coli cells with different localization of fluorescent signal.

FIG. 19: Dynamics of RNA localization and concentration changes within a single bacterium. (a) Time-lapse fluorescent micrographs of E. coli cells expressing complementing complex at 30 min intervals. Phase-contrast micrographs did not change during the duration of the experiment. (b) Total fluorescence changes measured in two single cells. (c) Changes in fluorescence distributions in real-time in a single bacterial cell measured along the long axis of the cell.

FIGS. 20A and 20B: Re-assembly of functional EGFP supported by DNA duplex formation. 20A: Outline of experiments. Purified α- and β-fragments of EGFP with Cys residues at the C- and N-termini, respectively, were biotinylated with N-[6-(biotinamido)hexyl]-3′-(2′-pyridyldithio)propionamide (HPDP-biotin, Pierce), purified and incubated with equimolar amounts of streptavidin. These complexes were then separately incubated with equimolar amounts of two complementary 21-nt long oligonucleotides. The yield of complexes in each step was verified by gel shift assay and was found to be close to 100%. Then two protein-oligonucleotide chimeras were mixed in equimolar concentrations. 1B: Fluorescent emission spectra of native (top) and re-assembled EGFP with appended 21 bp DNA duplex (bottom), excitation at 480 nm. Fluorescent spectra were measured in Na-phosphate buffer, pH 7.4, 150 mM NaCl, 1 mM EDTA.

FIG. 21: Mg2+ ions affect differently fluorescence of native and reconstructed EGFP.

FIGS. 22A and 22B: FIG. 22A: Fast kinetics of fluorescence increase upon mixing together α- and β-fragments of EGFP with appended complementary 21-nt long oligonucleotides. FIG. 22B: Kinetic pathway of chromophore formationin S65T with t½ of all the steps (from Zimmer, 2002).

FIGS. 23A-4B: Folded α-fragment of EGFP may contain pre-formed chromophore. FIG. 23A: Backbone representation of ten aligned typical folded structures of α-fragment from discrete molecular dynamics (DMD) simulations (Dokholyan, unpublished). Chromophore-forming amino acids are shown in blue. Note the tight packing of the N-terminal major part of the fragment and very flexible C-terminal part of the remaining fragment due to the small number of contacts with the rest of the molecule. FIG. 23B: Alignment of the folded α-domain and full-length EGFP. The full-length EGFP is colored yellow and the α-domain is colored blue. Chromophore-forming residues (#62-70) are shown in red. FIG. 23C: The average root-mean-square-deviation (RMSD) of each residue in the α-fragment of EGFP compared to amino acids in full-size EGFP. The RMSD values are calculated from DMD simulations at low temperatures when the protein is folded, The chromophore-forming amino acids are in a shaded area and their deviation is ≦2 Á.

FIGS. 24A-24D: α-fragment of EGFP contains pre-formed chromophore. FIG. 24A: Absorbance spectrum of EGFP, FIG. 24B: Absorbance of α- and β-fragments of EGFP, FIG. 24C: Fluorescence spectra of EGFP (blue—excitation, pink—emission), FIG. 24D: Fluorescence spectra of α-fragment (blue—excitation, pink—emission). Equimolar concentrations of all proteins were subjected to spectral analyses.

FIGS. 25-25B: Absorbance of the chromophore-containing peptide isolated from GFP by partial proteolysis (FIG. 25A) and of the chemically synthesized chromophore at acidic/neutral pH (FIG. 25B) (from Niwa et al., 1996).

FIG. 26: Two possible arrangements of nucleic acid interactions as support for protein complementation assay.

FIG. 27: Purification of beta-fragment of EGFP (beta-cys) expressed in E. coli as chimera with self-splitting intein. The protein in lane 6 is pure β-subunit of EGFP obtained after self-splitting of intein.

FIG. 28: Purification of the alpha-fragment of EGFP expressed in E. coli as chimera with self-splitting intein. Protein in lane 5 is pure α-subunit of EGFP.

FIG. 29: Conjugation of the proteins with SH-containing oligonucleotides is almost 100% complete. Analysis using non-denaturing PAGE. After conjugation, the proteins are linked to oligonucleotides bearing negative charge. Therefore, modified proteins move faster than unmodified (compare lane 1 and 2, and 3 and 4). If oligonucleotide is biotinylated, it can make a complex with streptavidin. Such complex also moves faster than streptavidin alone (compare lanes 6 and 7). From this data we conclude that the efficiency of oligonucleotide coupling with protein is close to 100%.

FIG. 30: Fluorescence of the combined alpha+A and beta+B shows reconstruction of the active EGFP, while in the absence of oligonucleotides there is no active EGFP.

FIG. 31: Schematics of protein complementation with restoration of protein enzymatic activity. The principle is similar to FIG. 6. Re-assembly of the enzyme is dependent on interaction of RNA with RNA-binding protein. An active re-assembled enzyme is splitting its substrate, which results in a chromogenic or fluorogenic product. Signal is thus amplified due to the enzymatic activity of the protein.

### DETAILED DESCRIPTION OF THE INVENTION

The inventors have discovered a novel method for rapid detection of RNA within a living cell which can be detected with a high signal to background ratio that enables high sensitivity of detection. The present invention comprises compositions and methods for the sensitive detection of nucleic acids, for example RNA and DNA in vitro and in vivo in real time. In particular, the present invention comprises a method using a detector molecule comprising a split-detector protein into at least two polypeptide fragments which are attached to nucleic acid binding motifs, for example RNA-binding motifs, which may function independently or cooperate to bind a single nucleic acid binding site. Re-association of the detector protein fragments into a functional detector protein will only occur in the presence of a target nucleic acid molecule as a result of interaction of the RNA-binding motifs with their cognate binding site(s) on the nucleic acid. This interaction will bring together the complementary polypeptide fragments of the detector protein, allowing for signal detection.

This invention, provides an innovative method for visualizing nucleic acids, such as RNA, in vivo, is based on protein complementation, meaning the re-association of two or more polypeptide protein fragments into an active protein. In particular, the split polypeptides are in an active configuration, yet inactive alone and therefore their association with the complementary polypeptide fragment immediately forms an active detector protein. In this case, complementation takes place only if it is supported by additional nucleic acid/protein interactions. In one embodiment, high-affinity and high-specificity protein/RNA aptamer interactions are described. An aptamer is expressed as a recognition tag within the target RNA, while the nucleic acid binding motif is synthesized as two inactive fragments fused to the fragments of the split detector protein. Interaction of nucleic acid binding motif fragments with the aptamer brings the detector protein fragments together, which results in enzymatic activity or fluorescence of the re-assembled detector protein inside within the cell.

In one embodiment, the methods described herein can be used for the real time detection of nucleic acids. A detector construct comprising a nucleic acid sequence encoding a detector molecule, the detector molecule comprising one polypeptide fragment of a detector protein conjugated with a nucleic acid binding motif and at least one other polypeptide fragment of the detector protein conjugated to a nucleic acid binding motif is described. In addition, the assay utilizes a reporter construct, encoding a reporter molecule comprising a nucleotide of interest and a nucleic acid binding sequence. The nucleic acid binding sequence is recognized by the nucleic acid binding motifs of the detector molecule. This recognition allows for the detector protein to be reconstituted and detected in real time and immediately. The detector protein does not have activity in the absence of target nucleic acid and thus is highly sensitive and exhibits low background. In addition, the fragmentation of the detector protein is designed so that on recognition in the presence of target nucleic acid sequences the activity is immediately detectable.

In one embodiment of the present invention the nucleic acid is RNA. Any RNA can be detected, selected from a group comprising mRNA, and miRNA. Alternatively, the nucleic acid is DNA.

The assay of the present invention describes as a nucleic acid binding motif. In one embodiment, the motif may be associated with the detector protein in a way in which the nucleic acid binding motif is split into a polypeptide fragment, and one fragment is conjugated with one fragment of the detector protein and rest of the nucleic acid polypeptide fragments are conjugated with one or more other fragments of the detector protein. In this embodiment, the binding of the motif is coordinated and each fragment must be present to bind to one nucleic acid binding sequence.

In an alternative embodiment, the nucleic acid binding motif that is associated with one polypeptide fragment of the detector protein may be a full-length motif that is independent from the other nucleic acid binding motif which is conjugated with one or more other fragments of the detector protein. For example, the nucleic acid binding protein may be a small multi-domain nucleic acid binding protein, therefore each domain associated with one or more fragments of the detector protein. In this embodiment, the binding of the nucleic acid binding motifs to their cognate nucleic acid sequences in the reporter molecule (or native nucleic acid) is independent.

Expression of Constructs in Cell

In one embodiment of the present invention, the detector and reporter molecules are expressed within a cell in vivo. To accomplish this, the nucleic acid sequences encoding the detector and reporter molecules may be inserted into the cell by any suitable method known by persons skilled in the art, e.g. transformation or transfection. In one embodiment the constructs may, for example, be on a single nucleic acid sequence. In an alternative embodiment, for example, they may be on two constructs (one comprising the reporter construct and one comprising the detector construct). Such constructs may be co-transformed or co-transfected into the cell. Without being bound by theory, it is proposed that the co-transformed/co-transfected constructs may recombine during transformation/transfection with the result that both are integrated at the same site in the cell's genomic DNA.

Alternatively, the detector and reporter constructs may alone or in combination, be stably expressed within a cell. Stable clones producing high levels of recombinant protein are obtained after transfection of cells with an expression vector encoding the desired genes of interest and a dominant genetic marker. Methods for stable, integration into a variety of cell types are known to those of skill in the art.

In one embodiment, the nucleic acid may encode the split-polypeptide fragments (comprising the detector protein and nucleic acid binding motif) that reconstitute to form the active detector protein. In one such embodiment, the split-polypeptide fragments may, for example, be linked by an internal ribosome entry site (IRES). An IRES allows for the production of a single transcript from two or more separate genes which can be translated into corresponding separate products due to the presence of an additional ribosome entry site(s) on the transcript. In an alternative embodiment, the split-polypeptide fragments and/or detector proteins and/or nucleic acid binding motifs may be encoded by one nucleic acid, with splittable sites between each nucleic acid sequence to enable separation of the desired polypeptides. As a non-limiting example, a nucleic acid could comprise a sequence encoding the active detector protein comprising at least two nucleic acid binding motifs, where the sequence encoding the detector protein comprises a splittable site to enable generation of split-polypeptide fragments of the detector protein each comprising a nucleic acid binding motif. In such an embodiment, the splittable site can enable the separation of the split-polypeptide fragments by any means known to persons skilled in the art, for example but not limited to, enzymatic cleavage, chemical cleavage; heat cleavage, acid cleavage, radiation cleavage, photocleavage etc. In addition, the detector construct and reporter construct may also be encoded on a single construct, with the separate components linked by an IRES.

Methods to introduce nucleic acid sequences into cells are known by persons skilled in the art, and include for example the detector and reporter constructs may be introduced into the cell by multiple means including vectors, viral vectors, and non-viral means. Non-viral means include without limitation, fusion, electroporation, biolistics, transfection, lipofection, protoplast fusion, calcium phosphate transfection, microinjection, pressure-forced entry, naked DNA etc. or any other means known any person skilled in the art.

The term “vectors” used interchangeably with “plasmid” refer to a nucleic acid molecule capable of transporting another nucleic acid to which it has been linked. Vectors capable of directing the expression of genes and/or nucleic acid sequence to which they are operatively linked are referred to herein as “expression vectors”. In general, expression vectors of utility in recombinant DNA techniques are often in the form of “plasmids” which refer to circular double stranded DNA loops which, in their vector form are not bound to the chromosome. Other expression vectors can be used in different embodiments of the invention, for example, but are not limited to, plasmids, episomes, bacteriophages or viral vectors, and such vectors may integrate into the host's genome or replicate autonomously in the particular cell. Other forms of expression vectors known by those skilled in the art which serve the equivalent functions can also be used. Expression vectors comprise expression vectors for stable or transient expression encoding the DNA.

In one embodiment, the detector and reporter construct may be introduced by homologous recombination, where it is desired that a construct be integrated at a particular locus. For example, one can delete and/or replace an endogenous gene at the same locus or elsewhere with the nucleic acid construct of this invention. For homologous recombination, the nucleic acid is cloned into specific vectors, including but not limited to; Ω or O-vectors, see, for example, Thomas and Capecchi, cell, (1987), 51; 503-512, Mansour et al, nature, (1988) 336; 348-352; and Joyner et al, nature (1989) 338; 153-156.

Vectors comprising useful elements such as bacterial or yeast origins of replication, selectable and/amplifiable markers, promoter/enhancer elements for the expression in prokaryotes or eukaryotes, and mammalian expression control elements etc, may be used to prepare stocks of nucleic acid constructs and for carrying out transfections are well known in the art, and many are commercially available.

In some embodiments, viral vectors may be used to introduce the nucleic acid sequences of the detector construct and nucleic acid construct, and drive their expression. Viral vectors refer to the use of viruses, or virus-associated vectors as carriers of the nucleic acid construct into the cell. Constructs may be integrated and packaged into non-replicating, defective viral genomes like Adenovirus, Adeno-associated virus (AAV), or Herpes simplex virus (HSV) or others, including reteroviral and lentiviral vectors, for infection or transduction into cells. The vector may or may not be incorporated into the cells genome. The constructs may include viral sequences for transfection, if desired. Alternatively, the construct may be incorporated into vectors capable of episomal replication, e.g EPV and EBV vectors.

For split-polypeptide fragments of the detector construct which have a polypeptides as the nucleic acid binding moiety, the entire split-polypeptide fragment and nucleic acid binding moiety molecule can be encoded by a single construct, including the polypeptide portion, a linker and the nucleic acid binding moiety polypeptide. This construct can either be expressed in the cell or microinjected into the cell. These constructs can also be used for in vitro detection of a nucleic acid of interest.

Aptamers

Aptamers are relatively short RNA or DNA oligonucleotides, which bind ligands and are isolated in vitro using selection procedure called SELEX (systematic evolution of ligands by exponential enrichment) (Tuerk & Gold, 1990; Ellington & Szostak, 1990). Because selection procedure is driven by binding of ligands, aptamers bind their ligands with high affinity and fold into secondary structures which are optimized for ligand binding (Herman & Patel, 2000). In this respect aptamers resemble antibodies by selectively binding corresponding ligand from complex chemical or biological mixtures.

Aptamers are particularly useful in the methods of the present invention. For example, the detector construct may be constructed so that nucleic acid binding motifs comprise aptamer binding sequences and the reporter construct to comprise aptamers. Conversely, the detector construct may comprise aptamers and the reporter construct may comprise aptamer binding sequences.

Thus, in one embodiment of the present invention, the aptamers can function after expression in cells by using vectors that encode an RNA oligonucleotide that comprises the aptamer such that the aptamer can be expressed without flanking sequences that can substantially interfere with the aptamer function. In one such embodiment, this is achieved with an RNA oligonucleotide that comprises the aptamer flanked by two self-cleaving ribozymes. As demonstrated in U.S. Patent Publication 20050222400, when the oligonucleotide is expressed, the aptamer is released upon cleavage of the self-cleaving ribozymes, and the aptamer that is released has short flanking sequences that are remnants of the self-cleaving ribozymes, which are highly unlikely to interfere with the proper folding of the aptamer sequence, as opposed to prior art methodologies. Without being limited to any particular mechanism, it is believed that these short flanking sequences do not substantially interfere with the aptamer function.

Since it is envisioned that the RNA oligonucleotide is, in some embodiments, a transcript from a DNA template, the RNA oligonucleotide can be any length that can be produced by transcription. Other elements, such as additional aptamers, ribozymes, coding regions, leader sequences, etc., can also be part of the RNA oligonucleotide, provided those elements do not substantially interfere with the function of the aptamer or the self-cleaving ribozymes.

The aptamer oligonucleotide of such an embodiment can be any useful aptamer now known or later developed. The aptamer may be targeted to a target that is inside a cell. One type of such a target is a component (i.e., a native constituent) of the cell. However, in an alternative embodiment the aptamer is targeted to an engineered component of a cell. Any cellular component that could be usefully affected by aptamer binding is envisioned as a potential target of the aptamers of the present invention. Non-limiting examples of such cellular components include proteins such as enzymes, structural proteins, ion channel proteins, electron transport proteins, ribozyme components, lipoproteins and transcription factors; proteoglycans; glycoproteins; polysaccharides; nucleic acids; lipids; and small molecules such as steroids.

Methods to design and synthesize aptamers and aptamer binding sequences are known to those of skill in the art.

Aptamer-Nucleic Acid Binding Protein Pair.

Any combination of aptamer-protein pair can be used in the invention. For example, in one embodiment, the nucleic acid binding proteins of the detector molecule can be transcription factors or proteins involved in RNA splicing. An example of a detector molecule is eIF4A which binds to a 58 nucleotide (nt) apatamer of the reporter molecule (Oguro et al, 2003) (see Examples 1-8). In other embodiments, the nucleic acid binding proteins and aptamer pairs include binary aptamer-peptide pairs and are shown in Table 1 and Example 11. In addition, other examples of aptamer and protein pairs, in particular RNA-protein partners, which are to be encompassed in the present invention include but are not limited to; (i) MS2 coat protein-RNA stem-loop (Sawata & Taira, 2003; Valegard et al., 1997); (ii) TAR-Tat BIV-1 stem-loop (Roy et al., 1990; Comolli et al., 1998); (iii) 3 repeats of the G3/C3 stem-loop shown to work as transcriptional activators (Jarrell & Ptashne, 2003); (iv) 3 repeats of the aptamer (Yamamoto et al., 2000); (v) eIF4A-87-nt long aptamer, which can be trimmed to 58 bases, the best Kd=27 nM (Oguro et al., 2003).

Nucleic Acid Binding Motifs.

The nucleic acid binding motif can be any polypeptide or protein or peptide molecule that can be coupled to another molecule, such as a polypeptide, and are capable of binding to a target nucleic acid in close proximity.

Other embodiments of the invention provide nucleic acid binding moieties which are polypeptides. The polypeptide can be any polypeptide with a high affinity for the target nucleic acid. In this embodiment, the target nucleic acid can be a double-stranded, triple-stranded, or single-stranded DNA or RNA. In some embodiments, the polypeptides is a peptide, less than 100 amino acids, or a full length protein. The polypeptide's affinity for the target nucleic acid can in the low nanomolar to high picomolar range. Polypeptides can include polypeptides which contain zinc fingers, either natural or designed by rational or screening approaches. Examples of zinc fingers include Zif 2g8, Sp1, finger 5 of Gfi-1, finger 3 of YY1, finger 4 and 6 of CF2II, and finger 2 of TTK (PNAS (2000) 97: 1495-1500; Biol Chem (20010 276 (21): 29466-78; Nucl Acids Res (2001) 29 (24):4920-9; Nucl Acid Res (2001) 29(11): 2427-36). Other polypeptides include polypeptides, obtained by in vitro selection, that bind to specific nucleic acids sequences. Examples of such aptamers include platelet-derived growth factor (PDGF) (Nat Biotech (2002) 20:473-77) and thrombin (Nature(1992) 355: 564-6. Yet other polypeptides are polypeptides which bind to DNA triplexes in vitro; examples include members of the heteronuclear ribonucleic particles (hnRNP) proteins such as hnRNP K, L, E1, A2/B1 and I (Nucl Acids Res (2001)₂₉(11): 2427-36).

The nucleic acid binding moiety of each split-polypeptide nucleic acid motif polypeptide can be any molecule which allows binding to a target nucleic acid. In some embodiments, the nucleic acid binding moiety includes nucleic acids, nucleic acid analogues, and polypeptides. In one embodiment, the nucleic acid binding moiety is an oligonucleotide. The nucleic acid binding moiety of a given pair of activated split-polypeptide fragment can be of the same kind of molecule, for example oligonucleotides, or they can be different, for example one split-polypeptide of a pair comprise an active protein can have an oligonucleotide nucleic acid binding moiety, and the other member of the pair can have a polypeptide nucleic acid binding moiety.

Detector Proteins

The split-polypeptide fragments of the detector protein, (which is part of the detector molecule), can be any polypeptide which associate when brought in to close proximity to generate an active protein, which can be detected by any means which allows recognition of the assembled active protein but not the individual polypeptides. For example, the two polypeptides may re-associate to generate a protein with enzymatic activity, to generate a protein with chromogenic or fluorogenic activity, or which create a protein recognized by an antibody. Furthermore, they are designed so that they are in the active state and primed (i.e. in a ready-state) for reconstitution of the active protein in order to minimize any lag time that is traditionally seen with protein complementation in vitro and in vivo.

In one embodiment the activated split-polypeptide fragments of the detector molecule are fluorescent proteins. In such an embodiment, one of the split fluorescent protein fragments are active in that one of the fragments contains a mature preformed chromophore that is primed and in the ready-state for immediate fluorescence upon complementation with its cognate activated split-fluorescent fragment(s).

In a preferred embodiment of the present invention the detector protein is a fluorescent protein, as a non-limiting example, enhanced green fluorescent protein (EGFP). Alternatively, the fluorescent protein may be a yellow fluorescent protein (YFP), an enhanced yellow fluorescent protein (EYFP), a blue fluorescent protein (BFP), an enhanced blue fluorescent protein (EBFP), a cyan fluorescent protein (CFP), an enhanced cyan fluorescent protein (ECFP) or a red fluorescent protein (dsRED) or any other natural or genetically engineered fragment thereof, where one fragment in the reconstituted fluorescent protein contains a mature preformed chromophore. All of the above mentioned fluorescent proteins and fragments thereof that result in a fluorescing fluorescent protein are encompassed for use in the present invention. Also encompassed are those fluorescent proteins known to those of skill in the art, and fragments and genetically engineered proteins thereof.

Alternatively, the detector protein is an enzyme that allows for signal amplification. The enzyme may be, for example, beta-lactamase, beta-galactosidase, beta-glucosidase, beta-glucuronidase, chloramphenicol acetyl transferase. In a specific embodiment the detector enzyme is beta-lactamase.

In another embodiment of the present invention the detector protein fragments are designed so that they are activated immediately when reconstituted. Furthermore, they are designed so that they are primed for reconstitution in order to minimize any lag time that is traditional seen with fluorescent proteins.

In some embodiments, the cognate non-fluorescent polypeptide fragment which combines with the mature chromophore-containing split-fluorescent fragment can comprise of more than one active non-fluorescent fragment. Such activated non-fluorescent polypeptides are usually produced by splitting the coding nucleotide sequence of one fluorescent protein at an appropriate site and expressing each nucleotide sequence fragment independently. The activated split-fluorescent protein fragments may be expressed alone or in fusion with one or more protein fusion partners.

In one embodiment of the invention, the reconstituted active protein comprises of activated split-EGFP fragments, wherein the first fragment is an N-terminal fragment of EGFP comprising a continuous stretch of amino acids from amino acid number 1 to approximately amino acid number 158. A C-terminal cysteine may be added to this fragment to aid in the conjugation of various nucleic acid binding motifs post expression. Another activated split-EGFP fragment is a continuous stretch of amino acids from approximately amino acid number 159 to amino acid number 239. A N-terminal cysteine may also be added. Amino acid 1 is meant to indicate the first amino acid of EGFP. Amino acid 239 is meant to indicate the last amino acid of the GFP. All residues are numbered according to the numbering of wild type A. victoria GFP (GenBank accession no. M62653; SEQ ID NO 7) and the numbering also applies to equivalent positions in homologous sequences. Thus, when working with truncated GFPs (compared to wild type GFP) or when working with GFPs with additional amino acids, the numbering must be altered accordingly.

In alternative embodiments, the reassembled fluorescent protein may comprise activated split fluorescence fragments from different and spectrally distinct fluorescent proteins. The reconstituted active fluorescent protein may have a distinct and/or unique spectral characteristics depending on the activated split-fluorescent fragments used for complementation. For example, multicolor fluorescence complementation has been achieved by reconstituting fragments from different fluorescent proteins for multicolor biomolecular fluorescence complementation (multicolor BiFC) (see Hu et al, Nature Biotechnology, 2003; 21; 539-545; Keippola, 2006, 7; 449-456, Hu, et al, Protein-Protein Interactions (Ed. P. Adams and E. Golemis), Cold Spring Harbor Laboratory Press. 2005, herein incorporated by reference in its entirety) Encompassed for use in the present invention are the use of activated split-fluorescent fragments from multiple fluorescent proteins for multicolor real-time fluorescence, wherein one of the fragments contains a pre-formed mature chromophore.

In one embodiment, the fluorescent protein is detectable by flow cytometry, fluorescence plate reader, fluorometer, microscopy, fluorescence resonance energy transfer (FRET), by the naked eye or by other methods known to persons skilled in the art. In an alternative embodiment, fluorescence is detected by flow cytometry using a florescence activated cell sorter (FACS) or time-lapse microscopy.

In another example, the marker is an enzyme, giving rise to chromogenic/fluorogenic product.

In another embodiment of the invention, the activated split-polypeptide fragments associated in close proximity to form an assembled, active enzyme, which can be detected using an enzyme activity assay. Preferably, the enzyme activity is detected by a chromogenic or fluorogenic reaction. In one preferred embodiment, the enzyme is dihydrofolate reductase (DHFR) or β-lactamase.

In another embodiment, the enzyme is dihydrofolate reductase (DHFR). For example, Michnick et al. have developed a “protein complementation assay” consisting of N- and C-terminal fragments of DHFR, which lack any enzymatic activity alone, but form a functional enzyme when brought into close proximity. See e.g. U.S. Pat. Nos. 6,428,951, 6,294,330, and 6,270,964, which are hereby incorporated by reference. Methods to detect DHFR activity, including chromogenic and fluoregenic methods, are well known in the art.

In alternative embodiments, other split polypeptides can be used. For example, enzymes that catalyze the conversion of a substrate to a detectable product. Several such systems for split-polypeptide reassemblies include, but are not limited to reassembly of; β-galactosidase (Rossi et al, 1997, PNAS, 94; 8405-8410); dihyrofolate reductase (DHFR) (Pelletier et al, PNAS, 1998; 95; 12141-12146); TEM-1′-lactamase (LAC) (Galarneau at al, Nat. Biotech. 2002; 20; 619-622) and firefly luciferase (Ray et al, PNAS, 2002, 99; 3105-3110 and Paulmurugan et al, 2002; PNAS, 99; 15608-15613). For example, split β-lactamase has been used for the detection of double stranded DNA (see Ooi et al, Biochemistry, 2006; 45; 3620-3525). Encompassed for use in the present invention are the use of activated split polypeptide fragments for real-time signal detection, wherein the fragments are in a fully folded mature conformation enabling rapid signal detection upon complementation.

In one embodiment, the detector protein that allows signal amplification can be used, for example the beta-lactamase system with the cell permeable pro-fluorescent beta-lactamase substrate, cephalosporin beta-lactam (CCF2) (Zlokarnik et al., 1998; Galarneau et al., 2002; Wehrman et al., 2002) which allows for about a 100-fold signal amplification (Zlokarnik et al., 1998; Galarneau et al., 2002).

In another embodiment of the invention, association of activated split-polypeptide fragments can form an assembled protein which contains a discontinuous epitope, which may be detected by use of an antibody which specifically recognizes the discontinuous epitope on the assembled protein but not the partial epitope present on either individual polypeptide. One such example of a discontinuous epitope is found in gp120 of HIV. These and other such derivatives can readily be made by the person of ordinary skill in the art based upon well known techniques, and screened for antibodies that recognize the assembled protein by neither protein fragment on its own.

In another embodiment of the invention, the activated split-polypeptides can be molecules which interact to form an assembled protein. For example, the molecules may be protein fragments, or subunits of a dimer or multimer.

The nucleic acid sequence and codons encoding the split-polypeptide fragments of interest may be optimized, for example, converting the codons to ones which are preferentially used in a desired system. For example in mammalian cells. Optimal codons for expression of proteins in non-mammalian cells are also known in the art, and can be used when the host cell is a non-mammalian cell (for example in insect cells).

The activated split-polypeptides of the present invention can comprise any additional modifications which are desirable. For example, in one embodiment, the activated split-polypeptides can also comprise a flexible linker, which is coupled to a nucleic acid binding moiety.

The detector proteins may be conjugated to the nucleic acid binding motif by any means known to persons skilled in the art. In one non-limiting example, the detector protein is conjugated to the nucleic acid binding motif by gene fusion. The term “conjugate” used herein refers to the attachment of two or more proteins joined together to form one entity. The proteins may be attached together by linkers, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat sensitive linkers.

The term “fusion protein” refers to a recombinant protein of two or more proteins. Fusion proteins can be produced, for example, by a nucleic acid sequence encoding one protein is joined to the nucleic acid encoding another protein such that they constitute a single open-reading frame that can be translated in the cells into a single polypeptide harboring all the intended proteins. The order of arrangement of the proteins can vary. As a non-limiting example, the nucleic acid sequence encoding the split-detector polypeptide fragment is fused in frame to an end, either the 5′ or the 3′ end, of a nucleic acid encoding the nucleic acid binding motif. In this manner, on expression of the gene, the split-detector protein is functionally expressed and comprises a fused, to the N-terminal or C-terminal end, a nucleic acid binding motif. Modification of the detector and/or fluorescent protein is such that the functionality of the detector and/or fluorescent protein remains substantially unaffected by fusion of the nucleic acid binding motif to the detector and/or fluorescent protein. In one embodiment, split-polypeptide fragments of EGFP are fused in-frame at the carboxyl terminal with split-eIF4A fragments or RNA binding peptides.

The term “linker” refers to any means to join two or more proteins by means other than the production of a fusion protein. A linker can be a covalent linker or a non-covalent linker. Examples of covalent linkers include covalent bonds or a linker moiety covalently attached to one or more of the proteins to be linked. The linker can also be a non-covalent bond, e.g an organometallic bond through a metal center such as platinum atom, For covalent linkages, various functionalities can be used, such as amide groups, including carbonic acid derivatives, ethers, esters, including organic and inorganic esters, amino, urethane, urea and the like. For example, to provide for linking, the nucleic acid binding motif and/or the fluorescent protein can be modified by oxidation, hydroxylation, substitution, reduction etc. to provide a site for coupling. It will be appreciated that modification which do not significantly decrease the function of the nucleic acid binding motif and/or the detector protein, for example the fluorescent protein.

Expression Vectors

Construction of vectors for recombinant expression of the detector and reporter constructs for use in the invention may be accomplished using conventional techniques which do not require detailed explanation to one of ordinary skill in the art. For review, however, those of ordinary skill may wish to consult Maniatis et al., in Molecular Cloning: A Laboratory Manual, Cold Spring Harbor Laboratory, (NY 1982).

Briefly, construction of nucleic acid sequence encoding the detector molecule and reporter molecule employs standard ligation techniques. For analysis to confirm correct sequences in nucleic acid constructs generated, the ligation mixtures may be used to transfect/transduce a host cell and successful genetically altered cells may be selected by antibiotic resistance where appropriate.

Vectors from the transfected/transduced cells are prepared, analyzed by restriction and/or sequenced by, for example, the method of Messing, et al., (Nucleic Acids Res., 9: 309-, 1981), the method of Maxam, et al., (Methods in Enzymology, 65: 499, 1980), the Sanger dideoxy-method or other suitable methods which will be known to those skilled in the art.

Expression of a gene is controlled at the transcription, translation or post-translation levels. Transcription initiation is an early and critical event in gene expression. This depends on the promoter and enhancer sequences and is influenced by specific cellular factors that interact with these sequences. The transcriptional unit of many prokaryotic genes consists of the promoter and in some cases enhancer or regulator elements (Banerji et al., Cell 27: 299 (1981); Corden et al., Science 209: 1406 (1980); and Breathnach and Chambon, Ann. Rev. Biochem. 50: 349 (1981)). For retroviruses, control elements involved in the replication of the retroviral genome reside in the long terminal repeat (LTR) (Weiss et al., eds. The molecular biology of tumor viruses: RNA tumor viruses, Cold Spring Harbor Laboratory, (NY 1982)). Moloney mouse leukemia virus (MLV) and Rous sarcoma virus (RSV) LTRs contain promoter and enhancer sequences (Jolly et al., Nucleic Acids Res. 11: 1855 (1983); Capecchi et al., In: Enhancer and eukaryotic gene expression, Gulzman and Shenk, eds., pp. 101-102, Cold Spring Harbor Laboratories (NY 1991). Other potent promoters include those derived from cytomegalovirus (CMV) and other wild-type viral promoters.

The practice of the present invention employs, unless otherwise indicated, conventional techniques of cell biology, cell culture, molecular biology, transgenic biology, microbiology, recombinant DNA and immunology, which are within the skill of the art. Such techniques are explained fully in the literature. See, for example, Molecular Cloning A Laboratory Manual, 2^{nd} Ed., ed. By Sambrook, Fritsch and Maniatis (Cold Spring Harbor Laboratory Press: 1989); DNA Cloning, Volumes I and II (D. N. Glover ed., 1985); Oligonucleotide Synthesis (M. J. Gait ed., 1984); Mullis er al, U.S. Pat. No. 4,683,195; Nucleic Acid Hybridization (B. D. Hames & S. J. Higgins eds. 1984); Transcription and Translation (B. D. Hames & S. J. Higgins eds. 1984); Culture of Animal Cells (R. I. Freshley, Alan R. Liss, Inc., 1987); Immobilized cells and Enzymes (IRL Press, 1986); B. Perbal, A Practical Guide to Molecular Cloning (1984); the treatise, Methods in Enzymology (Academic Press, Inc., N.Y.); Gene Transfer vectors for Mammalian Cells (J. H. Miller and M. P. Calos eds., 1987, Cold Spring Harbor Laboratory); Methods in Enzymology, Vols. 154 and 155 (Wu et al, eds.), Immunochemical methods In Cell and Molecular Biology (Mayer and Walker, eds., Academic Press, London, 1987); Handbook of Experimental Immunology, Volumes I-IV (D. M Weir and C. C. Blackwell eds., 1986); Manipulating the Mouse Embryo (Cold Spring Harbor Press: Cold Spring Harbor, N.Y., 1986).

Additional background information and general guidance to the practitioner with respect to the design, assembly, incorporation into plasmid, and transfection of constructs for such protein conjugates or fusion proteins and regulatory sequences is available for the following published international patent applications: WO94/18317; WO95/02684; WO95/24419 and WO 96/41865 and the contents of which are incorporated herein by reference.

Promoter and enhancer regions of a number of non-viral promoters have also been described (Schmidt et al., Nature 314: 285 (1985); Rossi and decrombrugghe, Proc. Natl. Acad. Sci. USA 84: 5590-5594 (1987)). Methods for maintaining and increasing expression of transgenes in quiescent cells include the use of promoters including collagen type I (1 and 2) (Prockop and Kivirikko, N. Eng. J. Med. 311: 376 (1984); Smith and Niles, Biochem. 19: 1820 (1980); de Wet et al., J. Biol. Chem., 258: 14385 (1983)), SV40 and LTR promoters.

According to one embodiment of the invention, the promoter is a constitutive promoter selected from the group consisting of: ubiquitin promoter, CMV promoter, JeT promoter, SV40 promoter, Elongation Factor 1 alpha promoter (EF1-alpha), chick beta-actin, PGK, MT-1 (Metallothionin).

Also encompassed in the present invention are inducible/repressible promoters. Non-limiting examples of these constructs are the “Tet-On”, “Tet-Off”, and Rapamycin-inducible promoter, which are encompassed in the present invention.

In addition to using viral and non-viral promoters to drive transgene expression, an enhancer sequence may be used to increase the level of transgene expression. Enhancers can increase the transcriptional activity not only of their native gene but also of some foreign genes (Armelor, Proc. Natl. Acad. Sci. USA 70: 2702 (1973)). For example, in the present invention collagen enhancer sequences are used with the collagen promoter 2 (I) to increase transgene expression. In addition, the enhancer element found in SV40 viruses may be used to increase transgene expression. This enhancer sequence consists of a 72 base pair repeat as described by Gruss et al., Proc. Natl. Acad. Sci. USA 78: 943 (1981); Benoist and Chambon, Nature 290: 304 (1981), and Fromm and Berg, J. Mol. Appl. Genetics, 1: 457 (1982), all of which are incorporated by reference herein. This repeat sequence can increase the transcription of many different viral and cellular genes when it is present in series with various promoters (Moreau et al., Nucleic Acids Res. 9: 6047 (1981).

Transgene expression may also be increased for long term stable expression using cytokines to modulate promoter activity. Several cytokines have been reported to modulate the expression of transgene from collagen 2 (I) and LTR promoters (Chua et al., connective Tissue Res., 25: 161-170 (1990); Elias et al., Annals N.Y. Acad. Sci., 580: 233-244 (1990)); Seliger et al., J. Immunol. 141: 2138-2144 (1988) and Seliger et al., J. Virology 62: 619-621 (1988)). For example, transforming growth factor (TOF), interleukin (IL)-I, and interferon (INF) down regulate the expression of transgenes driven by various promoters such as LTR. Tumor necrosis factor (TNF) and TGF 1 up regulate, and may be used to control, expression of transgenes driven by a promoter. Other cytokines that may prove useful include basic fibroblast growth factor (bFGF) and epidermal growth factor (EGF).

In one embodiment, a collagen promoter with the collagen enhancer sequence (Coll (E)) can also be used to increase transgene expression by suppressing further any immune response to the vector which may be generated in a treated brain notwithstanding its immune-protected status.

In another embodiment, the vector may comprise further sequences such as a sequence coding for the Cre-recombinase protein, and LoxP sequences. Another way of ensuring temporary expression of the detector and/or reporter constructs is through the use of the Cre-LoxP system which results in the excision of part of the inserted DNA sequence either upon administration of Cre-recombinase to the cells (Daewoong et al, Nature Biotechnology 19:929-933) or by incorporating a gene coding for the recombinase into the virus construct (Pluck, Int J Exp Path, 77:269-278). Incorporating a gene for the recombinase in the virus construct together with the LoxP sites and a structural gene (a neublastin in the present case) often results in expression of the structural gene for a period of approximately five days.

Transgenic Organisms

In one embodiment of the present invention, a transgenic organism is encompassed that expresses a reporter and detector construct for the real time detection of nucleic acids in vivo. The transgenic organisms, or animals, may carry the transgene in all their cells or in some, but not all cells, i.e., mosaic animals. The transgene can be integrated as a single transgene or in tandem, e.g., head to head tandems, or head to tail, or tail to tail, or as multiple copies. Double, triple, or multimeric transgenic animals may preferably comprise at least two or more transgenes. In a preferred embodiment, the animal comprises the a detector construct comprising two halves of the EGFP transgene operably linked to a nucleic acid binding motif and a transgene encoding a nucleic acid binding sequence and a gene of interest.

Where one or more genes encoding a protein are used as transgenes, it may be desirable to operably link the gene to an appropriate regulatory element, which will allow expression of the transgene. Regulatory elements, e.g., promoters, enhancers, (e.g., inducible or constitutive), or polyadenylation signals are well known in the art. Regulatory sequences can be endogenous regulatory sequences, i.e., regulatory sequences from the same animal species as that in which it is introduced, as a transgene. The regulatory sequences can also be the natural regulatory sequence of the gene that is used as a transgene.

A transgene construct described herein may include a 3′ untranslated region downstream of the DNA sequence. Such regions can stabilize the RNA transcript of the expression system and thus increase the yield of desired protein from the expression system. Among the 3′ untranslated regions useful in the constructs of this invention are sequences that provide a polyA signal. Such sequences may be derived, e.g., from the SV40 small t antigen, or other 3′ untranslated sequences well known in the art. The length of the 3′ untranslated region is not critical but the stabilizing effect of its polyA transcript appears important in stabilizing the RNA of the expression sequence.

A transgene construct may also include a 5′ untranslated region between the promoter and the DNA sequence encoding the signal sequence. Such untranslated regions can be from the same control region from which promoter is taken or can be from a different gene, e.g., they may be derived from other synthetic, semi-synthetic, or natural sources.

Antisense nucleic acids may also be used in the transgene construct of the present invention. For example, an antisense polynucleotide sequence (complementary to the DNA coding strand) may be introduced into the cell to decrease the expression of a “normal” gene. This approach utilizes, for example, antisense nucleic acid, ribozymes, or triplex agents to block transcription or translation of a specific mRNA, either by masking that mRNA with an antisense nucleic acid or triplex agent, or by cleaving it with a ribozyme. Alternatively, the method includes administration of a reagent that mimics the action or effect of a gene product or blocks the action of the gene. The use of antisense methods to alter the in vitro translation of genes is well known in the art (see e.g., Marcus-Sekura, 172 ANAL. BIOCHEM. 289-95, 1988).

The transgene constructs described herein may be inserted into any suitable plasmid, bacteriophage, or viral vector for amplification, and may thereby be propagated using methods known in the art, such as those described by Maniatis et al. (MOLECULAR CLONING: A LABORATORY MANUAL, Cold Spring Harbor, N.Y., 1989). A construct may be prepared as part of a larger plasmid, which allows the cloning and selection of the constructions in an efficient manner as is known in the art. Constructs may be located between convenient restriction sites on the plasmid so that they may be easily isolated from the remaining plasmid sequences for incorporation into the desired mammal.

The methods of the present invention also relate to the production of transgenic animals or cells by the introduction of exogenous DNA into an oocyte using retroviral vectors. Retroviral vectors can be used to transfer genes efficiently into host cells by exploiting the viral infectious process (Kim et al., 4 ANIM. BIOTECHNOL. 53-69, 1993; Kim et al., 35 MOL. REPROD. DEV. 105-13, 1993; Haskell and Bowen, 40 MOL. REPROD. DEV. 386-90, 1995; Chan et al., 95 PROC. NATL. ACAD. SCl. USA 14028-33, 1998; Krimpenfort et al., 1991; Bowen et al., 50 BIOL. REPROD. 664-68, 1994; Tada et al., 1 TRANSGENICS 535-40, 1995).

Applications

The method of the invention can be used in multiple applications known by persons skilled in the art. One important embodiment of the present invention is a reporter assay for the real time detection of RNA in vivo. In one such embodiment, cells of interest stably express a detector molecule which does not produce a signal from the detector protein in the absence of target RNA and thus does not fluoresce or show activity. The cells can also express an inducible reporter molecule comprising a binding site for the detector molecule's nucleic acid binding motif(s) and a gene of interest, which when expressed within a cell, the detector molecule will bind the target RNA and facilitate detector protein reconstitution and production of an active protein and signal. This allows for the real time detection of RNA expression in vivo and is exemplified in FIGS. 10-11 and 18-19 and Examples 9-11.

In an alternative embodiment, the cells of interest can express the reporter construct which is operatively linked to a promoter of interest. When the promoter is activated within the cell by certain stimuli, the binding site(s) for the target nucleic acid sequence becomes available and binding of the nucleic acid binding protein of the detector molecule facilitates the association of the detector polypeptide fragments and formation of the active detector protein which can be immediately detected. This embodiment allows for promoter function to be studied in real time in vivo. For example, the regulation of a gene can be studied in real time by transfecting a plasmid expressing a promoter driving an RNA binding site and analyzing any baseline detector construct activity. In a related embodiment, the cells are contacted with i) a compound or compounds; or ii) subjected to a variety of conditions that may alter the activity of the promoter, thereby increasing, decreasing or causing no change in the transcription of the RNA. This alteration is immediately detected by the detector construct.

Alternatively, the methods of the invention provide methods to detect the presence of target nucleic acid in vivo, even in the absence of a reporter construct. In such an embodiment, for example, the nucleic acid, for example DNA and/or RNA, comprises the target nucleic acid sequence which binds the nucleic acid binding motif of the detector molecule. In such an embodiment, depending on the design of the nucleic acid components of the detector molecule, naive or non-modified nucleic acids can be detected in vivo.

In a similar embodiment, a “tet-on” or “tet-off” system may be utilized in the methods of the present invention. For example, cells of interest may be created to stably express the detector construct of the present invention. In addition, the cells may also stably express a tet-on or tet-off regulatable reporter construct enabling controlled transcription of the target binding site(s) and gene of interest for the detector molecules nucleic acid binding motifs). For example, in a tet-off system, the cells would immediately express the gene of interest and the reporter construct would be active and immediately detectable. Upon the addition of tet, the transcription of the reporter construct (comprising the gene of interest and target nucleic acid binding site) is shut off and only the transcripts that have been already transcribed can be analyzed in real time. In this tet-off example one could analyze the stability of RNA, such as for example, its half-life, in vivo in real time by detecting the diminishing reporter fluorescence or activity.

In an alternative embodiment, the use of a “tet-on” construct allows for the stable expression of a gene of interest in a living cell, whereby the addition of tet to the system turns on transcription of the reporter construct. The detector molecule of the present invention is then able to bind its target RNA and is immediately detectable. In this embodiment, RNA localization may be studied in real time as dictated by the person conducting the experiment.

In yet another embodiment of the present invention, the reporter assay is in vivo in an organism. For example but not limited to, animals, non-human animals, mouse, zebrafish, C. elegans, or frog may be created to express both a detector construct and a gene of interest with an RNA binding site that will bind to the detector construct. The expression of both constructs within the organism allows for a transcript of interest to be analyzed throughout development by taking samples from Day 1 embryo throughout adulthood. In one embodiment, the organism is a mammal. In one embodiment the mammal is a mouse, and in another embodiment the organism is a transgenic organism, for example but not limited to a transgenic mouse.

Alternatively, the compositions and methods of the present invention provide for the real time detection of RNA in an in vivo method similar to in situ hybridization, for example Fluorescence in situ Hybridization (FISH). In such an embodiment, a cell expressing a reporter construct comprising a RNA of interest with a RNA binding sequence that will bind to the nucleic acid binding motif of the detector molecule is permeablized, and the detector constructs of the invention are administered to the cell. If the RNA of interest is expressed, the detector construct will bind to the RNA binding sequences and the localization and abundance of RNA can be determined. The advantages of this system over traditional in situ hybridization techniques are the ability to detect low abundance RNAs (due to the signal amplification attainable by a detector construct which utilizes an enzyme reporter) and the ability to finish the assay quickly and without radioactivity. The immediacy of the detector construct detection is highly desirable. Currently, traditional methods of performing in situ hybridization (e.g. with radioactivity) can take weeks to months before even knowing if the experiment worked or not. In addition, the RNA probes used in traditional in situ hybridization are difficult to prepare due to the ease of RNA degradation and the use of radioactivity. In the methods of the present invention there is no need for radioactivity or RNA probes. The detector constructs are easily prepared, for example, from bacteria, as is known to those of skill in the art. Such an assay is similar to immunohisto- or immunocyto-chemistry techniques whereby antibodies are used to detect protein in vitro. Here, RNA is detected.

Furthermore, the compositions and methods of the present invention may be used in embodiments to detect DNA in vivo in real time. For example, the detector construct of the present invention may comprise a detector protein (either fluorescent or enzymatic) that is split into at least two inactivated polypeptide fragments, each fragment associated with a DNA-binding motif. The polypeptide fragments, when brought together by the presence of target nucleic acid, in this case DNA, form a fully active detector protein, and can be immediately detected. For example, the methods of the present invention allow for the real time detection of replication of various loci within the genome. The DNA-binding motifs associated with the detector proteins in the detector construct are specific for various regions of the DNA and the replication of those loci can be detected in real time in vivo.

Similarly, one could detect chromosomes in real time in vivo using the methods and compositions of the present invention. For example, the detector construct may comprise a detector protein associated with telomerase binding proteins and expressed within cells. In this way, teleomeres can be detected in real time over the lifespan of the cell. The advantage of the compositions and methods presented herein is that the specificity is increased by providing a telomerase binding protein that is bisected into two halves, each half associated with one half of the detector protein. The coordinated binding of the two halves of the telomerase binding proteins to a single binding site ensures that there is little to no detector activity prior to target recognition.

In another embodiment of the present invention, a method to detect PCR products in real time is disclosed. Using the methods and compositions of the present invention the products of a PCR reaction are detected with higher specificity than is present available and in real time. In an example of this embodiment, a PCR primer is designed so that the amplification product of the reaction incorporates an aptamer that can be specifically recognized by a nucleic acid-binding motif present on a detector construct. As amplification progresses, the detector protein can be detected in real time. The present invention provides advantages over currently used techniques, such as SYBR green, because the detector construct is specific for PCR products and would not detect template DNA. Using this embodiment, RNA can be detected in real time by converting RNA to cDNA before conducting the PCR experiment. In a related embodiment, the nucleic acid binding motifs components of the detector molecule facilitate the reassembly of the split-detector molecule in the presence of PCR products, allowing for a novel method for immunoPCR in vivo. Also, in another embodiment, the nucleic acid binding components of the detector molecule can facilitate the reassembly of the split-detector molecule, and therefore signal, in the presence of nucleic acids in immunoRCA (rolling circle amplification) methods, resulting in high signal amplification in vivo.

In another embodiment of the present invention, a method for detecting polymorphisms, mutations or aberrant gene expression in an individual is disclosed. For example, the assay of the present invention can be used to detect DNA and/or RNA from an individual for the diagnosis of certain diseases, disorders or predisposition to such diseases and disorders. Accordingly, the methods of the present invention allow for the diagnosis and prognosis of various diseases and disorders if the detector constructs can be designed with nucleic acid-binding motifs specific for certain sequences that might be present in the genome of an individual with a disease or disorder.

In a related embodiment, the present invention allows for the real-time detection of gene mutations, polymorphisms, or aberrations in an individual. As an illustrative example, an aptamer could tag or transiently attach to a specific mutation or polymorphism site, which could be detected by the split-polypeptide molecule of the present invention that is designed so that the nucleic acid binding motifs of the detector molecule recognizes the attached aptamer associated to a gene of interest comprising specific mutations, polymorphisms or aberration one is trying to detect. Alternatively, a pool of molecules may be used whereby many mutations, polymorphisms, or aberrations may be detected. If the nucleic acid binding motif recognizes the attached target nucleic acid, such as an aptamer associated with the gene perbutation and/or alteration, protein complementation of the detector protein results and allows for sensitive detection due to the immediacy of signal and/or fluorescent production.

In one important embodiment, the molecule can be used for real-time detection of pathogens. In one embodiment, the molecule of the invention can be used to detect the presence of pathogen nucleic acid sequences and/or aberration in nucleic acid sequences as a result of presence of pathogen and/or pathogen nucleic acid. The pathogen can be a virus infection, fungi infection, bacterial infection, parasitic infection and other infectious diseases. Viruses can be selected from a group of viruses comprising of Herpes simplex virus type-1, Herpes simplex virus type-2, Cytomegalovirus, Epstein-Barr virus, Varicella-zoster virus, Human herpes virus 6, Human herpes virus 7, Human herpes virus 8, Variola virus, Vesicular stomatitis virus, Hepatitis A virus, Hepatitis B virus, Hepatitis C virus, Hepatitis D virus, Hepatitis E virus, Rhinovirus, Coronavirus, Influenza virus A, Influenza virus B. Measles virus, Polyomavirus, Human Papilomavirus, Respiratory syncytial virus, Adenovirus, Coxsackie virus, Dengue virus, Mumps virus, Poliovirus, Rabies virus, Rous sarcoma virus, Yellow fever virus, Ebola virus, Marburg virus, Lassa fever virus, Eastern Equine Encephalitis virus, Japanese Encephalitis virus, St. Louis Encephalitis virus, Murray Valley fever virus, West Nile virus, Rift Valley fever virus, Rotavirus A, Rotavirus B. Rotavirus C, Sindbis virus, Simian Immunodeficiency virus, Human T-cell Leukemia virus type-1, Hantavirus, Rubella virus, Simian Immunodeficiency virus, Human Immunodeficiency virus type-1, and Human Immunodeficiency virus type-2.

The target nucleic acid may also be useful for the detection of bacteria and eukaryotes in food, beverages, water, pharmaceutical products, personal care products, dairy products or environmental samples. Preferred beverages include soda, bottled water, fruit juice, beer, wine or liquor products. Assays developed will be particularly useful for the analysis of raw materials, equipment, products or processes used to manufacture or store food, beverages, water, pharmaceutical products, personal care products, dairy products or environmental samples.

In another related embodiment of the invention, the assembly of the activated split-polypeptide fragment forms an assembled detector protein which contains a discontinuous epitope, which may be detected by use of an antibody which specifically recognizes the discontinuous epitope on the assembled protein but not the partial epitope present on either individual polypeptide. One such example of a discontinuous epitope is found in gp120 of HIV. These and other such derivatives can readily be made by the person of ordinary skill in the art based upon well known techniques, and screened for antibodies that recognize the assembled protein by neither protein fragment on its own.

The target nucleic acid can be of human origin. The target nucleic acid can be DNA or RNA. The target nucleic acid can be free in solution or immobilized to a solid support.

In one embodiment, the target nucleic acid is specific for a genetically based disease or is specific for a predisposition to a genetically based disease. Said diseases can be, for example, beta-thalassemia, sickle cell anemia or Factor-V Leiden, genetically-based diseases like cystic fibrosis (CF), cancer related targets like p53 and p10, or BRC-1 and BRC-2 for breast cancer susceptibility. In yet another embodiment, isolated chromosomal DNA may be investigated in relation to paternity testing, identity confirmation or crime investigation.

In another embodiment, the present invention provides kits suitable for the method of the invention, in particular detecting the RNA in vivo. In one embodiment, the kits comprise the detector construct and the inducible reporter construct. In one embodiment the reporter construct and detector construct are on separate nucleic acid molecules. In another embodiment, they are both encoded in a biocistronic nucleic acid molecule. In a related embodiment, the split-polypeptide fragments are separated by an IRES site. In another embodiment, the polypeptide-fragments are encoded as one nucleic acid comprising a splittable site and can be split to form separate split-polypeptide fragments by means known by persons skilled in the art, for example but not limited to enzymatic cleavage; chemical cleavage, photocleavage, acid cleavage, thermal or heat cleavage etc. In another embodiment, the reporter molecule can be modified to add the practitioner's gene of interest, and in another embodiment, the expression of the reporter molecule is operatively linked to a tet-on or tet-off promoter. In another variant embodiment, the reporter construct, comprising the target nucleic acid sequence can be modified by the practitioner to be regulated by the practitioner's promoter of interest. In each of these embodiments, the kits comprise a choice of split polypeptides for the detector protein in the detector molecule, and also comprise instructions and reagents for detecting a signal from the detector protein. In some embodiments, the kits also comprise reagents suitable for capturing and/or detecting the present or amount of target nucleic acid in a sample. The reagents for detecting the present and/or amount of target nucleic acid can include enzymatic activity reagents or an antibody specific for the assembled protein. The antibody can be labeled.

The combination of protein complementation and an enzymatic reporter allows for a substantially reduced background and signal amplification. This results in an in vivo RNA detection technique capable of analyzing nucleic acids of low, moderate and high abundance.

In another embodiment, the present invention provides kits suitable for detecting the presence and/or amount of a target nucleic acid in vivo. The kits comprise at least a first probe coupled to a first molecule and a second probe coupled to a second molecule, wherein the probes can bind to a hybridization sequence in a target nucleic acid. Preferably, the probes are in vials. The kits also comprise reagents suitable for capturing and/or detecting the present or amount of target nucleic acid in a sample. The reagents for detecting the present and/or amount of target nucleic acid can include enzymatic activity reagents or an antibody specific for the assembled protein. The antibody can be labeled. Such kits may optionally include the reagents required for performing RCA reactions, immunoRCA, immunoPCR, such as DNA polymerase, DNA polymerase cofactors, and deoxyribonucleotide-5′-triphosphates. Optionally, the kit may also include various polynucleotide molecules, DNA or RNA ligases, restriction endonucleases, reverse transcriptases, terminal transferases, various buffers and reagents, antibodies and inhibitors of RNase and nuclease activity. These components are in containers, such as vials. The kits may also include reagents necessary for performing positive and negative control reactions, as well as instructions. Optimal amounts of reagents to be used in a given reaction can be readily determined by the skilled artisan having the benefit of the current disclosure.

In another embodiment, the methods of the invention can be used for protein complementation for multiple nucleic acid targets simultaneously. For example, protein complementation of complementary split-polypeptide fragments which have associated different nucleic acid binding motifs. The presence of one target nucleic acid will facilitate protein complementation of one active split-polypeptide fragment pair, while the presence of another target will facilitate protein complementation of another pair of activated split-polypeptide fragments, resulting in a different active protein and detectable signal. In such an embodiment, multiple nucleic acid targets can be detected simultaneously. In an alternative embodiment, simultaneous detection of target nucleic acids, such as RNA and DNA can be monitored by real-time protein complementation.

In a related embodiment, the multiple protein complementation using split-fluorescent protein fragments from different fluorescent proteins. In a related embodiment, the methods of the invention enable real-time detection and identification of specific target nucleic among a variety of other putative but different nucleic acid targets (see Hu et al, Nature Biotechnology, 2003; 21; 539-545; Kerppola, 2006, 7; 449-456, Hu, et al, Protein-Protein Interactions (Ed. P. Adams and E. Golemis), Cold Spring Harbor Laboratory Press. 2005, herein incorporated by reference in its entirety).

### DEFINITIONS

Unless stated otherwise, the following terms and phrases as used herein are intended to have the following meanings:

The term “detector construct” used herein is intended to encompass the nucleic acid sequence encoding the detector protein and nucleic acid binding motif which comprise the detector molecule.

The term “reported construct” used herein is intended to encompass the nucleic acid of interest (for example DNA or RNA) and the target nucleic acid sequence (for example RNA aptamer) which comprise the reporter molecule.

The term “eukaryote” or “eukaryotic organism” is intended to encompass all organisms in the animal, plant, and protist kingdoms, including protozoa, fungi, yeasts, green algae, single celled plants, multi celled plants, and all animals, both vertebrates and invertebrates. The term does not encompass bacteria or viruses. A “eukaryotic cell” is intended to encompass a singular “eukaryotic cell” as well as plural “eukaryotic cells,” and comprises cells derived from a eukaryote.

The term “vertebrate” is intended to encompass a singular “vertebrate” as well as plural “vertebrates,” and comprises mammals and birds, as well as fish, reptiles, and amphibians.

The term “mammal” is intended to encompass a singular “mammal” and plural “mammals,” and includes, but is not limited to humans; primates such as apes, monkeys, orangutans, and chimpanzees; canids such as dogs and wolves; felids such as cats, lions, and tigers; equids such as horses, donkeys, and zebras, food animals such as cows, pigs, and sheep; ungulates such as deer and giraffes; rodents such as mice, rats, hamsters and guinea pigs; and bears. Preferably, the mammal is a human subject.

The term “in vivo” as used herein is intended to encompass a live cell, present in an organism. When discussing a live cell outside the organism, the term “ex vivo” is typically used. A live cell can be any cell, form any organism, including multi-cellular organisms and single cell organisms such as yeast and bacteria.

The term “invasive” used herein, with respect to detecting nucleic acids in live cells refers to methods that detect nucleic acids using invasive methods, such as, for example, lipofectamine and microinjection. The term “non-invasive” therefore refers to methods to detect nucleic acids in live or living cells without such invasive procedures.

The terms “tissue culture” or “cell culture” or “culture” or “culturing” refer to the maintenance or growth of plant or animal tissue or cells in vitro under conditions that allow preservation of cell architecture, preservation of cell function, further differentiation, or all three. “Primary tissue cells” are those taken directly from tissue, i.e., a population of cells of the same kind performing the same function in an organism. Treating such tissue cells with the proteolytic enzyme trypsin, for example, dissociates them into individual primary tissue cells that grow or maintain cell architecture when seeded onto culture plates. Cell cultures arising from multiplication of primary cells in tissue culture are called “secondary cell cultures.” Most secondary cells divide a finite number of times and then die. A few secondary cells, however, may pass through this “crisis period,” after which they are able to multiply indefinitely to form a continuous “cell line.” The liquid medium in which cells are cultured is referred to herein as “culture medium” or “culture media.” Culture medium into which desired molecules, e.g., immunoglobulin molecules, have been secreted during culture of the cells therein is referred to herein as “conditioned medium.”

Cells also refer not to a particular subject cell but to the progeny or potential progeny of such a cell because of certain modifications or environmental influences, for example differentiation, such that the progeny mat not, in fact be identical to the parent cell, but are still included in the scope of the invention.

The cells used in the invention can be cultured cells, e.g. in vitro or ex vivo, as shown in the Examples herein. For example, cells cultured in vitro in a culture medium and environmental stimuli can be added to the culture medium. Alternatively, for ex vivo cultured cells, cells can be obtained from a subject, where the subject is healthy and/or affected with a disease. Cells can be obtained, as a non-limiting example, by biopsy or other surgical means know to those skilled in the art.

The term “IRES” refers to internal ribosome entry sites (see Kozak (1991) J. Biol. Chem. 266′19867-70) are sequences encoding consensus ribosome binding sites, and can be inserted immediately 5′ of the start codon and/or regulatory sequences or elsewhere 5′ of nucleic acid sequences encoding genes or marker genes to enhance expression of the downstream nucleic acid sequence. The desirably of, or need for, such modification may be empirically determined.

The term “polynucleotide” refers to any one or more nucleic acid segments, or nucleic acid molecules, e.g., DNA or RNA fragments, present in a nucleic acid or construct. A “polynucleotide encoding an gene of interest” refers to a polynucleotide which comprises the coding region for such a polypeptide. In addition, a polynucleotide may encode a regulatory element such as a promoter or a transcription terminator, or may encode a specific element of a polypeptide or protein, such as a secretory signal peptide or a functional domain.

A “nucleotide” is a monomer unit in a polymeric nucleic acid, such as DNA or RNA, and is composed of three distinct subparts or moieties: sugar, phosphate, and nucleobase (Blackburn, M., 1996). When part of a duplex, nucleotides are also referred to as “base” or “base pairs”. The most common naturally-occurring nucleobases, adenine (A), guanine (G), uracil (U), cytosine (C), and thymine (T) bear the hydrogen-bonding functionality that binds one nucleic acid strand to another in a sequence specific manner. “Nucleoside” refers to a nucleotide that lacks a phosphate. In DNA and RNA, the nucleoside monomers are linked by phosphodiester linkages, where as used herein, the term “phosphodiester linkage” refers to phosphodiester bonds or bonds including phosphate analogs thereof, including associated counter-ions, e.g., IT′, NW, Na′, and the like.

“Polynucleotide” or “oligonucleotide” refer to linear polymers of natural nucleotide monomers or analogs thereof, including double and single stranded deoxyribonucleotides “DNA”, ribonucleotides “RNA”, and the like. In other words, an “oligonucleotide” is a chain of deoxyribonucleotides or ribonucleotides, that are the structural units that comprise deoxyribonucleic acid (DNA) and ribonucleic acid (RNA), respectively. Polynucleotides typically range in size from a few monomeric units, e.g. 8-40, to several thousand monomeric units. Whenever a DNA polynucleotide is represented by a sequence of letters, such as “ATGCCTG,” it will be understood that the nucleotides are in 5′→3′ order from left to right and that “A” denotes deoxyadenosine, “C” denotes deoxycytidine, “G” denotes deoxyguanosine, and “T” denotes thymidine, unless otherwise noted.

“Watson/Crick base-pairing” and “Watson/Crick complementarity” refer to the pattern of specific pairs of nucleotides, and analogs thereof, that bind together through hydrogen-bonds, e.g. A pairs with T and U, and G pairs with C. The act of specific base-pairing is “hybridization” or “hybridizing”. A hybrid forms when two, or more, complementary strands of nucleic acids or nucleic acid analogs undergo base-pairing.

“Detection” refers to detecting, observing, or measuring a construct on the basis of the properties of a detection label.

The term “nucleobase-modified” refers to base-pairing derivatives of AGC, T, U, the naturally occurring nucleobases found in DNA and RNA.

The term “promoter” refers to the minimal nucleotide sequence sufficient to direct transcription. Also included in the invention are those promoter elements that are sufficient to render promoter-dependent gene expression controllable for cell-type specific, tissue specific, or inducible by external signals or agents; such elements may be located in the 5′ or 3′ regions of the native gene, or in the introns. The term “inducible promoter” refers to a promoter where the rate of RNA polymerase binding and initiation of transcription can be modulated by external stimuli. The term “constitutive promoter” refers to a promoter where the rate of RNA polymerase binding and initiation of transcription is constant and relatively independent of external stimuli. A “temporally regulated promoter” is a promoter where the rate of RNA polymerase binding and initiation of transcription is modulated at a specific time during development. All of these promoter types are encompassed in the present invention.

As used herein, a “promoter” or “promoter region” or “promoter element” used interchangeably herein, refers to a segment of a nucleic acid sequence, typically but not limited to DNA or RNA or analogues thereof, that controls the transcription of the nucleic acid sequence to which it is operatively linked. The promoter region includes specific sequences that are sufficient for RNA polymerase recognition, binding and transcription initiation. This portion of the promoter region is referred to as the promoter. In addition, the promoter region includes sequences which modulate this recognition, binding and transcription initiation activity of RNA polymerase. These sequences may be cis-acting or may be responsive to trans-acting factors. Promoters, depending upon the nature of the regulation may be constitutive or regulated.

The term “constitutively active promoter” refers to a promoter of a gene which is expressed at all times within a given cell. Exemplary promoters for use in mammalian cells include cytomegalovirus (CMV), and for use in prokaryotic cells include the bacteriophage T7 and T3 promoters, and the like.

The term “operatively linked” or “operatively associated” are used interchangeably herein, and refer to the functional relationship of the nucleic acid sequences with regulatory sequences of nucleotides, such as promoters, enhancers, transcriptional and translational stop sites, and other signal sequences. For example, operative linkage of nucleic acid sequences, typically DNA, to a regulatory sequence or promoter region refers to the physical and functional relationship between the DNA and the regulatory sequence or promoter such that the transcription of such DNA is initiated from the regulatory sequence or promoter, by an RNA polymerase that specifically recognizes, binds and transcribes the DNA. In order to optimize expression and/or in vitro transcription, it may be necessary to modify the regulatory sequence for the expression of the nucleic acid or DNA in the cell type for which it is expressed. The desirability of, or need of, such modification may be empirically determined.

The term “conjugate” refers to the attachment of two or more proteins joined together to form one entity. The proteins may attached together by linkers, chemical modification, peptide linkers, chemical linkers, covalent or non-covalent bonds, or protein fusion or by any means known to one skilled in the art. The joining may be permanent or reversible. In some embodiments, several linkers may be included in order to take advantage of desired properties of each linker and each protein in the conjugate. Flexible linkers and linkers that increase the solubility of the conjugates are contemplated for use alone or with other linkers are incorporated herein. Peptide linkers may be linked by expressing DNA encoding the linker to one or more proteins in the conjugate. Linkers may be acid cleavable, photocleavable and heat sensitive linkers. In some embodiments, “Conjugate” or “conjugated” also encompasses covalent, ionic, or hydrophobic interaction whereby the moieties of a molecule are held together and preserved in proximity.

In the present invention, an “aptamer” refers to a nucleic acid ligand artificially engineered to bind strongly and specifically with a particular target protein. A “modulate aptamer” refers to an aptamer wherein the core portion of the aptamer is designed such that it has a low Tm value and divided in two short oligonucleotide chains which bind to form a double strand only in the presence of a target protein.

### EXAMPLES

### Example 1

### Methods of Protein-Complementation Facilitated by Nucleic Acid Interactions

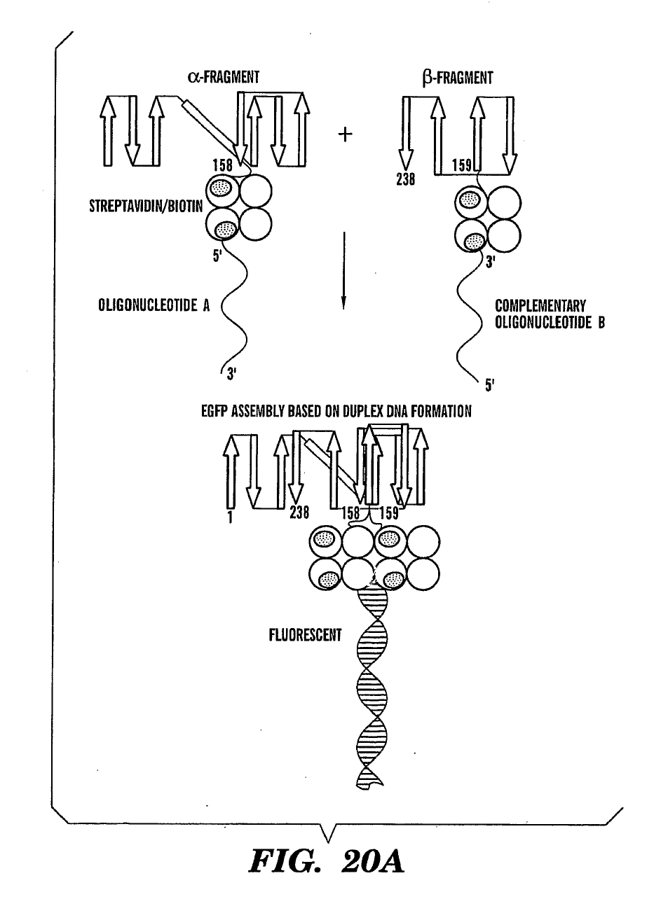

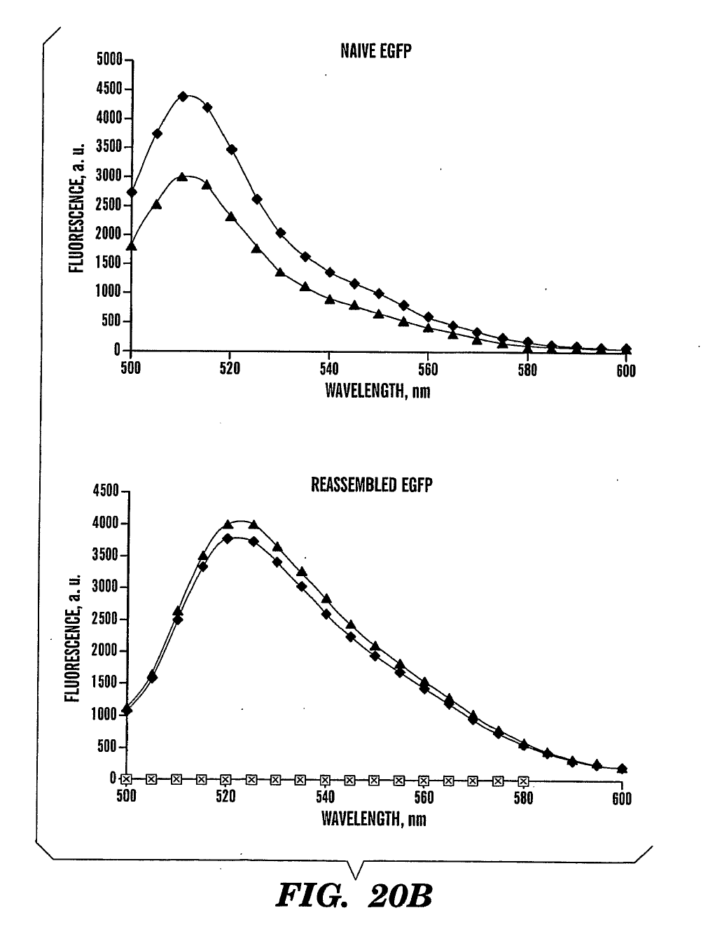

To show the workability of fast protein complementation facilitated by nucleic acid interactions, several experiments were performed in vitro. FIGS. 20a, 20b show how nucleic acid interaction discussed herein work. In these experiments, enhanced Green Fluorescent protein (EGFP) was chosen as a marker-protein for several numerous reasons. First, activity of EGFP is easily determined by the presence of characteristic fluorescence. Second, fluorescent proteins from the GFP family have already been successfully used as markers or detector proteins in several protein-protein complementation studies for example, Ozawa et al., 2000, Ozawa et al., 2001a,b; Ghosh et al., 2000; Hu et al., 2002; Hu & Kerppola, 2003; Remy & Michnick, 2004; Magliery et al., 2005 which demonstrate schemes to successfully split EGFP.

These studies showed that the loop between 153-161 amino acids in EGFP is a convenient site for splitting: protein insertions within this loop did not affect protein folding and chromophore formation in vivo (Ozawa et al., 2000, Ozawa et al., 2001 a,b; Ghosh et al., 2000; Hu et al., 2002; Hu & Kerppola, 2003; Remy & Michnick, 2004; Magliery et al., 2005). Also, importantly, it was shown that there is no spontaneous self-assembly of the fluorophore or chromophore when the two fragments were co-expressed in E. coli (Gosh et al., 2000; Hu et al., 2002; Magliery et al., 2005). Thus, to demonstrate two split polypeptide fragments of GFP could be directed to associate by nucleic acid interactions to form an active fluorescent protein, we designed two polypeptide fragments of EGFP were conjugated with complementary oligonucleotides to demonstrate that, duplex DNA formation of the coupled oligonucleotides facilitates the re-association of EGFP fragments to produce an active EGFP protein and development of strong fluorescence.

In these studies, EGFP was genetically split at position 158 into two parts, termed α- and β-fragments. For coupling with oligonucleotides, the α- and β-EGFP fragments were designed with extra cystein residues at the C- and N-terminus, respectively. They were expressed in E. coli as C-terminal fusions with intein and purified using intein self-splitting chemistry (NE Biolabs). The purified proteins were in vitro quantitatively biotinylated using convenient Cys-targeted biotin-HPDP chemistry (Pierce) to the conjugated oligonucleotides. The biotinylated proteins were tagged first with streptavidin followed by the oligonucleotides bearing biotin at the 5′- or 3′-end. In this way complementary 21 nt-long oligonucleotides with biotins on different ends were readily appended to the α- and β-fragments of EGFP through tetrameric streptavidin molecule (FIG. 20a). When these molecular chimeras were combined in equimolar amounts, the development of strong fluorescence revealed formation of the fluorophore with the emission spectrum resembling EGFP (FIG. 20b). In control experiment, biotinylated α- and β-fragments of EGFP fused with streptavidin but without complementary oligonucleotides were mixed together, and no significant fluorescence was detected (FIG. 20b). Fluorescence recovery somewhat varied from experiment to experiment with the maximal recovery close to 100% of fluorescence of the intact folded EGF, Thus, in most cases, 100% of GFP fluorescence can be restored by directed protein complementation.

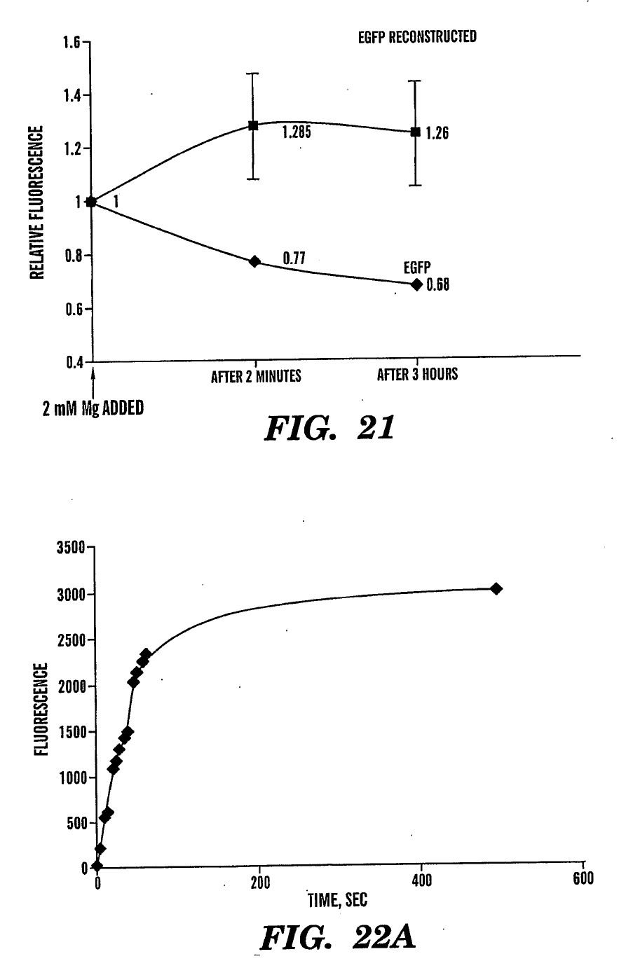

The fluorescent spectra of the reconstructed EGFP from reconstitution of the split EGFP fragments with appended oligonucleotides was somewhat different from those of the native EGFP. First, the emission and excitation maxima of the reconstructed protein (490/524) were shifted to the red compared to the native EGFP (488/507 nm, Zimmer, 2002) (FIG. 20b). This can be explained by the possible small differences in the conformation of the β-barrel structure in the reconstituted protein. Second, differences between the reconstructed complex and EGFP were apparent upon addition of Mg2+ ions. While fluorescence of the native protein was decreased about 30% upon addition of 2 mM Mg2+, fluorescence of the reconstructed EGFP was first increased about 30% and then gradually decreased (FIG. 21). The differing effect of Mg2+ can be explained by the presence of the duplex DNA appended to the reconstructed EGFP. Indeed, DNA duplex holds together two fragments of EGFP like a pincer grip, and because addition of Mg2+ ions makes DNA duplex more stable, this may result in higher stability of the re-assembled EGFP and consequently in initial increase of fluorescence. On the contrary, for native fluorescent proteins, the presence of bivalent metal cations in the vicinity of the chromophore is known to quench fluorescence (Richmond et al., 2000; Zimmer, 2002). Therefore, gradual decrease of fluorescence in the re-constructed EGFP is the result of two processes: stabilization of the complex due to higher stability of the DNA duplex in the presence of Mg2+ ions and fluorescence quenching.

### Example 2

### Detection Proteins for Directed Fast Protein Complementation

In example 1, the marker-protein or detector protein in the protein complementation is a fluorescent protein, namely, the enhanced green fluorescent protein (EGFP), which is a double mutant of the jellyfish Aequorea victoria GFP (F64L, S65T). Splitting of EGFP and other related fluorescent proteins at residues 154-158 has been successfully used in several studies designed to test protein/protein interactions in vivo (Ghosh et al., 2000; Hu & Kerppola, 2003; Remy & Michnick 2004; Magliery et al., 2005). These studies showed that the re-assembly of active EGFP from its fragments in vivo does not happen spontaneously but requires an additional protein/protein interaction (Maglieri et al., 2005). Additionally, it has been shown that EGFP re-assembly is quite tolerant to the size of interacting proteins: (Ghosh et al., 2000; Hu & Kerppola, 2003; Remy & Michnick 2004; Magliery et al., 2005).

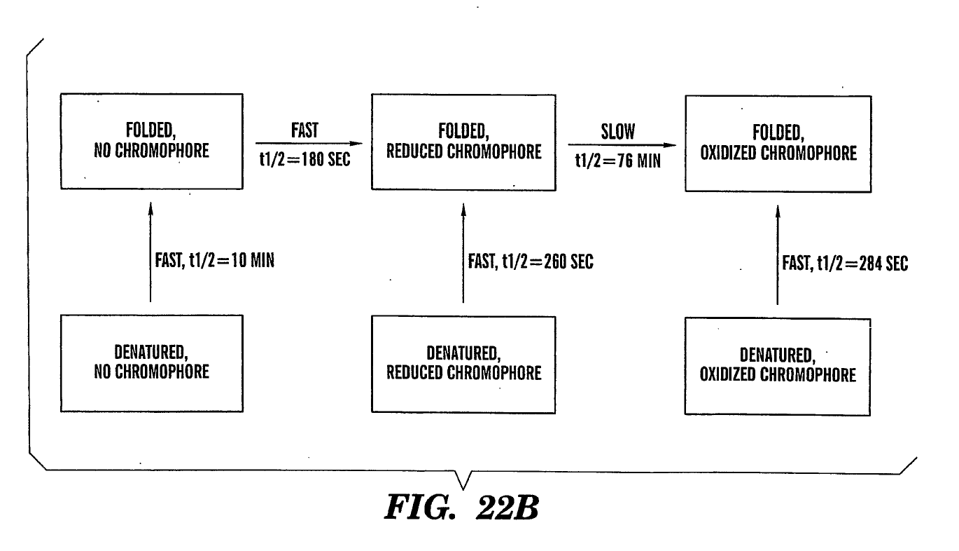

To use protein complementation for RNA localization studies, it is especially important that the kinetics of fragment re-assembly is fast enough. In our preliminary studies in vitro, an increase in fluorescence is caused by very fast complementation of activated split-EGFP fragments (FIG. 22A). This is possible because the α-fragment of EGFP contains a pre-formed mature chromophore, and therefore is in the activated conformation and can immediately form the active fluorescent protein on association with the complementary β-fragment of EGFP. This unique property of fast kinetics of EGFP re-assembly enables the monitoring of fast movements of target RNAs in vivo. To this end, we expressed two protein components of complementation system from one plasmid and after 2-3 hrs induced the synthesis of the target RNA with the aptamer-tag. In this way, we are able to monitor movements of a specific RNA.

It is also possible to use another marker-protein, such as, for example, variant of yellow fluorescent protein, Venus (F64L, M153T, V163A, S175G), since it has faster than EGFP rate of fluorophore maturation (Kₒₓ=8×10⁻³ s⁻¹ versus 1.5×10⁻⁴ s⁻¹) and higher quantum yield (Nagai et al. 2002). Additionally, this protein is more stable to photobleaching, and its spectral characteristics allow for better discrimination from autofluorescence of cellular proteins.

In another example, the marker is an enzyme, giving rise to chromogenic/fluorogenic product.

To develop a protein complementation system with signal amplification, we took advantage of the beta-lactamase system with a pro-fluorescent beta-lactamase substrate, cephalosporin beta-lactam (CCF2) (Zlokarnik et al., 1998; Galarneau et al., 2002; Wehrman et al., 2002) that is cell-permeable. It was shown that this enzymatic system allows for about 100-fold signal amplification (Zlokarnik et al., 1998; Galarneau et al., 2002). Quantitative in vivo analysis showed that as low as 50 molecules of beta-lactamase per Jurkat cell can be detected after 16 hr above the background. By decreasing time of exposure to the substrate, 20,000 molecules of beta-lactamase were quantitated. This sensitivity is substantially higher than the sensitivity achievable with fluorescent proteins, where 10⁵-10⁶ target mol/cell are necessary to be detected above background autofluorescence (Zlokarnik et al., 1998).

Therefore, a marker protein with enzymatic activity will generate higher signal than a fluorescent protein. However, spatial resolution may be partly lost in the enzymatic approach due to the diffusion of the small molecular weight colored reaction products. Thus, depending on the need of the particular experiment either one of those two systems (fluorescent or enzymatic) can be used.

To prove the possibility of protein complementation assisted by complementary nucleic acid interactions we choose the molecule of Enhanced Green Fluorescent Protein (EGFP) as a model and dissected it into two fragments, 1-158 and 159-234 aa. The corresponding genes were obtained by PCR using a plasmid containing full EGFP-1 gene (Clontech, PaloAlto, Calif.). Both fragments were engineered to have additional Cys residues at the C-terminus (alphaCys-fragment, 1-158) and at the N-terminus (betaCys-fragment, 159-234 aa). PCR products were cloned in the TWIN-1 vector (New England Biolabs, MA), as C-terminal fusions of Ssp DNAB intein. The structure of all the constructs was verified by sequencing.

The α-fragment of EGFP comprises a pre-formed chromophore. Though with some small spectral differences, up to 100% fluorescence can be restored by nucleic acid-based protein complementation. The kinetics of the DNA-templated EGFP re-assembly was surprisingly fast with the t½˜100 sec (see FIG. 22), being close to kinetics of renaturation of EGFP from denatured protein with mature chromophore (Reid & Flynn, 1997, Zimmer, 2000). Of note, typical chromophore maturation in fluorescent proteins requires hours (Zimmer, 2000, FIG. 22b). Based on the fast kinetic data, the methods of the invention enable the production of an EGFP α-fragment containing a mature chromophore. Since α-fragment is large enough containing 158 amino acids from the N-terminus (out of 239 amino acids total in EGFP), it has the potential for full chromophore formation.

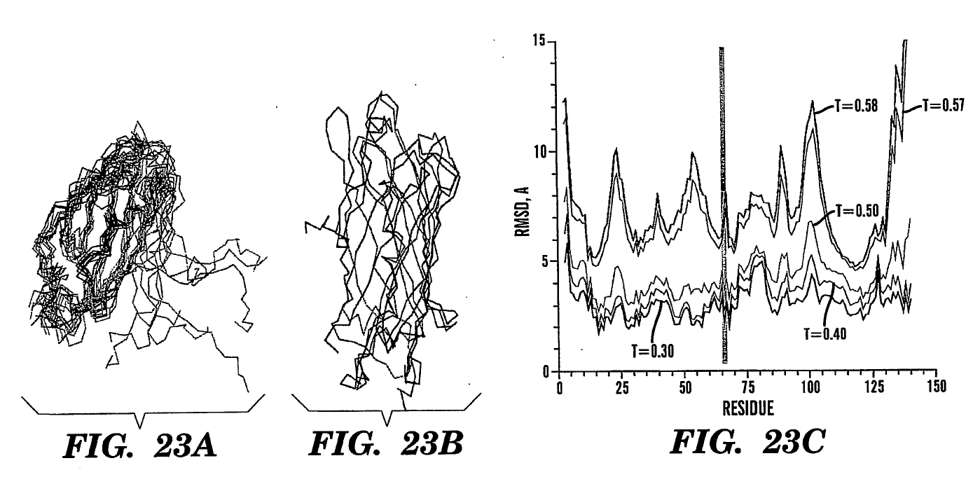

Molecular modeling supports the presence of a fully formed mature chromophores in the α-fragment of EGFP. In FIG. 23 a schematic representation of the structural conformation of the α-fragment indicates the α-fragment folds into very compact structure (except for its dangling C-terminal part), and that spatial coordinates of the chromophore-forming amino acids in α-fragment and in the full-size EGFP are very close (FIG. 23 b, c). This is also in line with the specific architecture of the polypeptide chain of fluorescent proteins having a strong ˜800 bend in the central helix, which brings in close proximity fluorophore-forming amino acids T66, Y67 and G68 (Barondeau et al., 2003; Donnely et al., 2001). The chromophore in the α-fragment, although fully formed lacks many important contacts with amino acids in the C-terminal β-fragment, which are present in the full-size EGFP, and it is exposed to solvent, thus lacking ability to develop strong fluorescence featured by the chromophore in the complete protein.

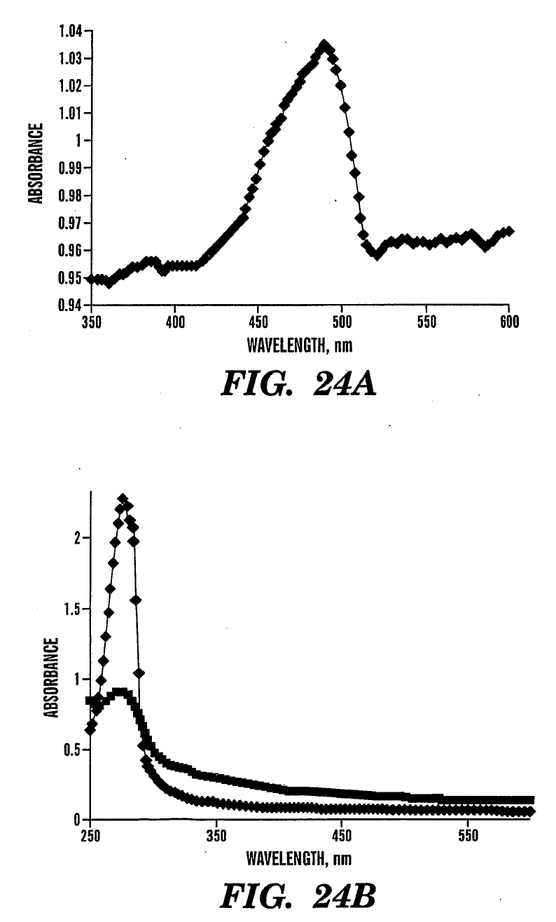

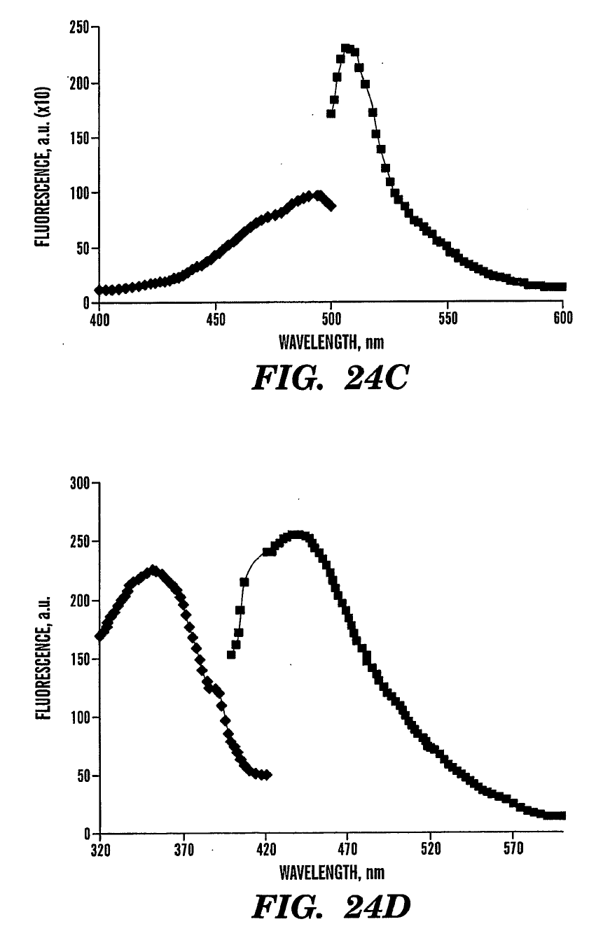

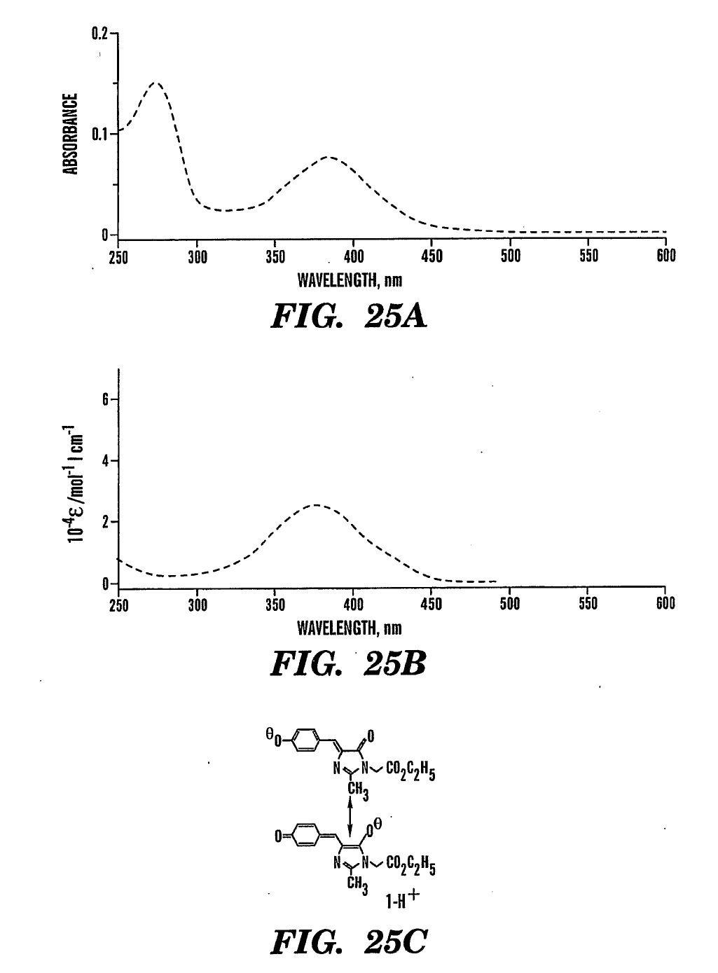

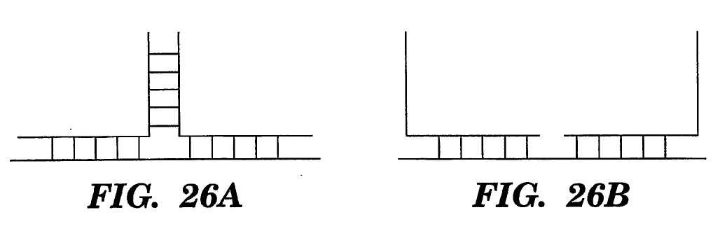

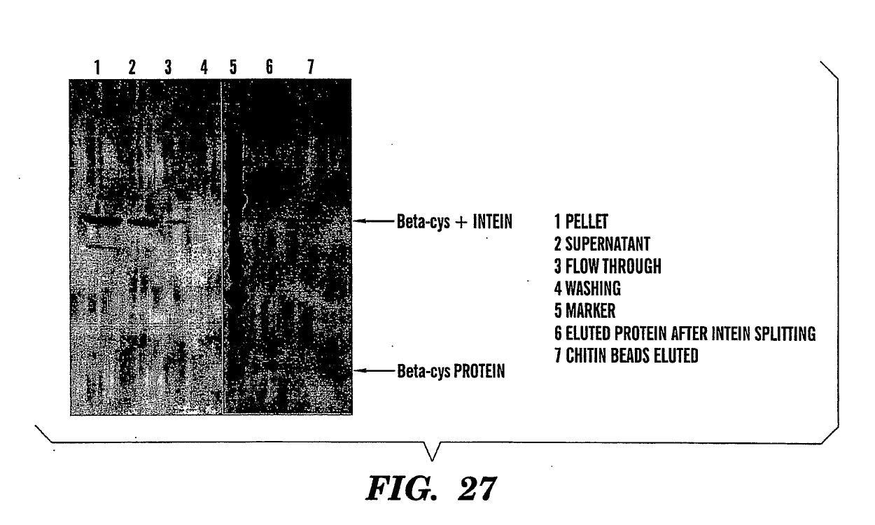

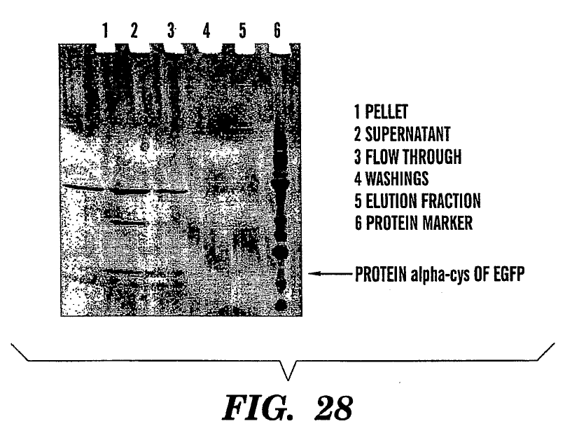

To directly establish the presence of the pro-fluorescent chromophore in the α-fragment, we analyzed absorbtion and fluorescent spectra of the α-fragment alone and compared them with the spectra of the full-size EGFP (FIG. 24). As a control, spectra of the β-fragment of EGFP and of two non-fluorescent proteins (streptavidin and chymotrypsinogen) were also recorded. Absorbance spectra of both α- and β-EGFP fragments are shown in FIG. 24b. As one can see, none of them has a characteristic maximum at 490 nm, which is visible in native EGFP (FIG. 24a). However, the α-fragment features rather higher absorbance in the region 300-400 nm that is absent in the β-fragment (FIG. 24b, insert), or in any non-fluorescent protein (not shown). Fluorescence spectra of the α-fragment though (FIG. 24d) clearly show characteristic maxima at 360 nm in excitation spectrum and at 460 nm in weak emission spectrum. These spectra are quite different from those of the full-length EGFP (FIG. 24c), however, they correspond with the spectra of the synthetic chromophore and the spectra of the short chromophore-containing peptide isolated from the GFP by partial proteolysis (FIG. 25) (Niwa et al., 1996). Note that the solvent-exposed chromophore (as it is in the α-fragment) should absorb light at 300-400 nm, but not at 450-500 nm (as in the full-size EGFP). In a separate experiment, the split-EGFP fragments produce a different spectra to fully formed naive structure EGFP when expressed in living E. coli in vivo. (FIG. 25). Thus produced in this way, the α- and β-fragment of EGFP are in an activated form, although non-fluorescent or inactive alone, they can re-associate to form an active fluorescent protein.

This, reassociation of the activated complementary EGFP fragments can in vitro and in vivo, and that nucleic acid interactions can facilitate the complementation of the EGFP fragments. The fast kinetics of fluorescent recovery or reconstitution is consistent with the fragments being in an activated state, which are only active on reconstitution with the corresponding complementary fragment. In particular, the α-fragment of EGFP contains a mature chromophore and therefore is in an activated state yet is inactive alone due to quenching of the mature chromophore. Addition of the EGFP β-fragment containing 81 C-terminal amino acids leads to the formation of the compact bi-partite protein structure, shielding the chromophore from the environment, resulting in fast and strong fluorescence development (provided that the two protein fragments are kept together by associated DNA-DNA or other nucleic acid complementary interactions).

These rather unexpected results are very important for the development of methods for fast-protein complementation in vivo. Using an α-fragment of EGFP already containing a mature chromophore and re-association of the α-fragment with the β-fragment results in the formation of an active EGFP protein that occurs within seconds. Expressing these split-EGFP or split-fluorescent fragments, where one fragment containing a pre-formed mature chromophores can be done prior to induction of the RNA component. The induction of RNA synthesis of the RNA component will facilitate fast-protein complementation of the fragments and the increase in fluorescence. This should permit one to follow the fate of particular RNA from the early moments of its expression.

The results showed that the incubation of the two fragments of EGFP bearing complementary oligonucleotides resulted in fluorescence emission spectrum (excitation max 490 nm) increase with the maximum at 524 nm, which is characteristic for EGFP spectrum. No changes in fluorescence spectra were found when EGFP fragments were mixed together without complementary nucleotides.

### Example 3

### Conjugation of Detector Protein and Nucleic Acid Binding Protein

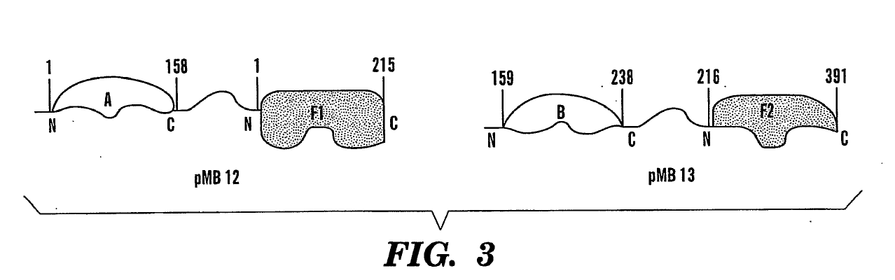

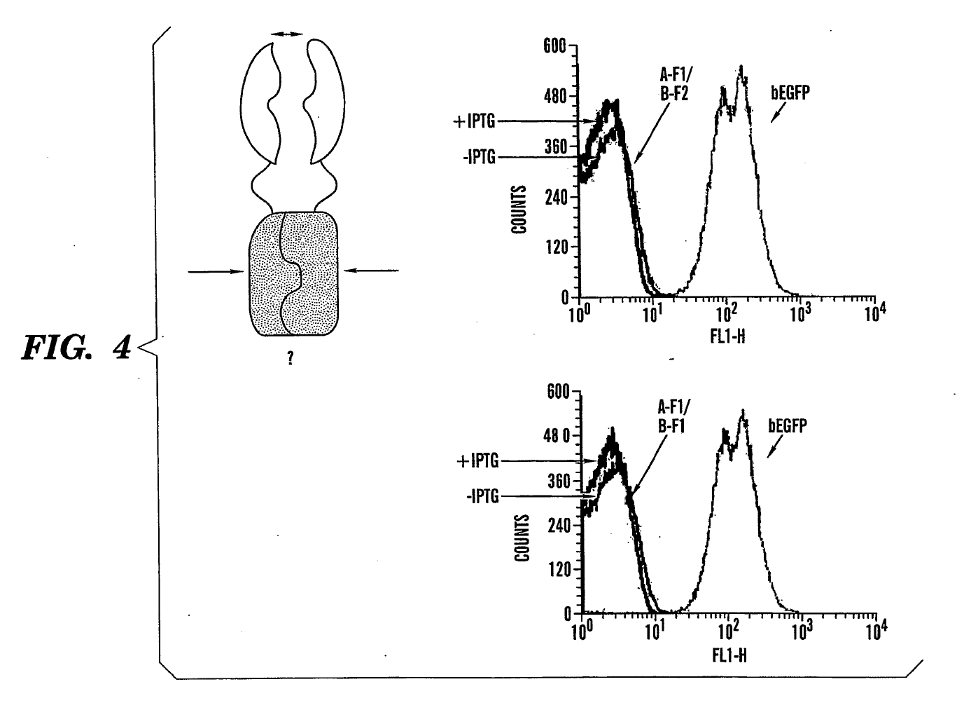

In this experiment we verified that expression of the two dissected protein chimeras will not result in reconstituted fluorescence in the absence of the interacting aptamer sequence. This experiment was performed in E. coli. Fragments of EGFP gene were obtained by PCR from the plasmid pEGFP (Clontech). Splitting of EGFP gene was in the same position as in experiments in vitro (1-158, 159-239). The plasmid containing full-size eIF4A (pGEX-4A1) was utilized. The F1 and F2 fragments of eIF4A were obtained by PCR, splitting was performed according to Oguro et al. (see FIG. 3). Two fusion proteins with an SG linker were inserted into two plasmids pETDuet 1 and pACYDuet-1 (Novagen) constructed for co-expression of two messages. These two plasmids have different origins of replication and different selective markers. The resulting plasmids pMB12 and pMB13 were co-expressed in E. coli strain BL21 (DE3) (Novagen). In pMB12, the first 158 amino acids of EGFP, termed A, were linked to the first 215 amino acids of the eukaryotic initiation factor protein 4A (eIF-4A), termed F1, through a (Ser-Gly)₅ peptide linker. Similarly, the peptide containing C-terminal 92 amino acids of EGFP, termed B, was linked to the second-half of eIF-4A, termed F2 in pMB13 (FIG. 3). Both constructs were under the control of the T7 promoter and were inducted with 1 mM IPTG. Fluorescence levels were measured by FACS and they were comparable to those in the non-induced cultures. (FIG. 4). As a positive control in this experiment we expressed full-length EGFP in vector pTWIN (NEB), and as a negative control-fragments of EGFP fused to the same F1 fragment of the eIF4A (A-F1, B-F1) (FIG. 4).

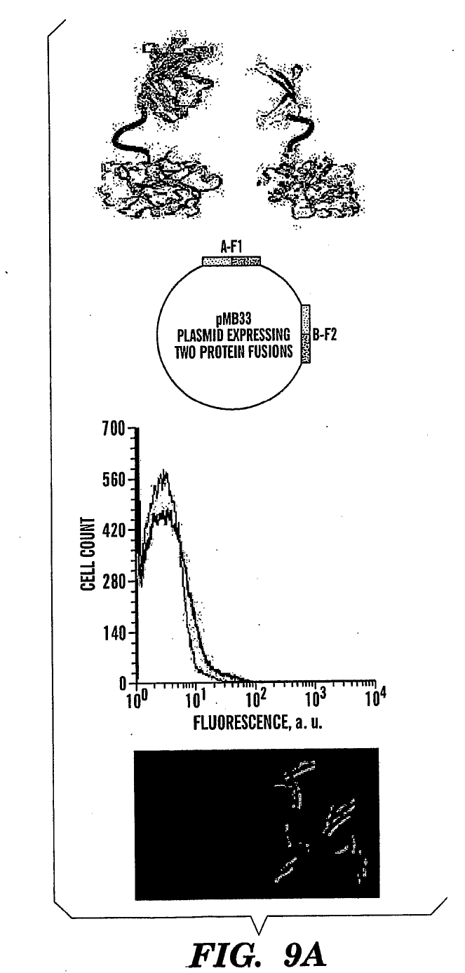

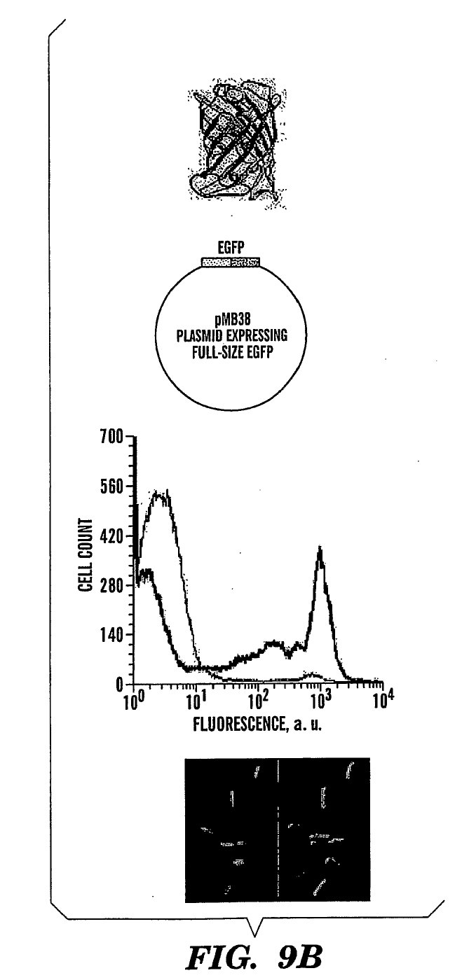

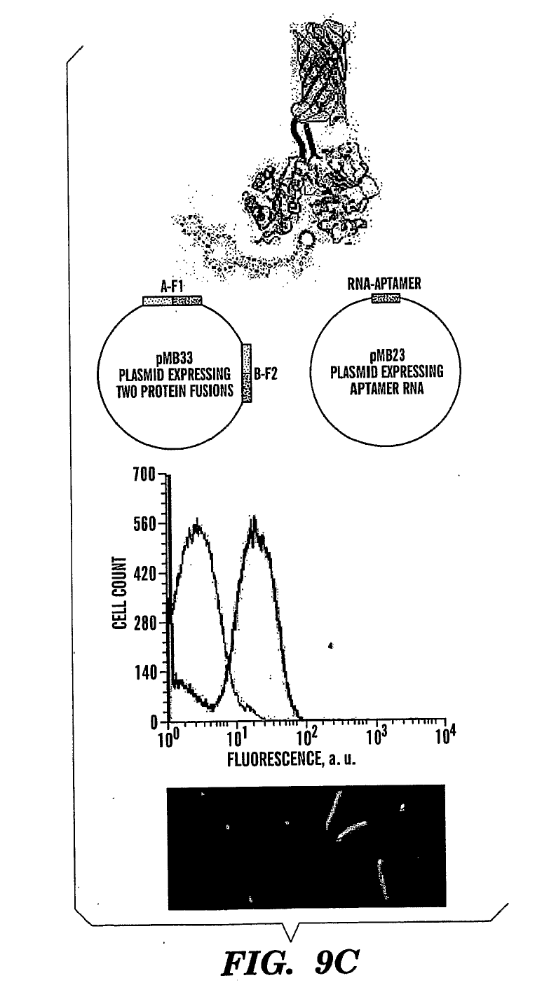

In another experiment, the A-EGFP-F1-eIF-4A and B-EGFP-F2-eIF-4A nucleic acids was expressed in the same plasmid termed pMP33, which was expressed in E. coli. The pMB33 plasmid is a derivative of pACYCDuet1 (Novagen), and is a bicistronic plasmid expressing both chimeric split-EGFP-eIF-4A fragment proteins, comprising the fragment of the marker protein (e.g., EGFP or enzyme) and the fragment of RNA-binding protein (e.g., MS2 coat protein, or eIF4a). The expressed chimeric split-fragments do not reassociate in absence of target RNA (FIG. 9a), but reassociate rapidly in the presence of target RNA to produce a functional EGFP marker protein (FIG. 9c).

Thus, nucleic acid interaction of the RNA-binding protein with the target RNA can facilitate fast protein complementation in vitro and in vivo and recovery of fluorescence with fast kinetics from the split EGFP fragments. This can be facilitated by either complementary binding of associated 21-bp oligonucleotide or RNA-binding protein with the nucleic acid.

### Example 4

### Choice of the Aptamer-Nucleic Acid Binding Protein Pair

In one embodiment of the invention, protein complementation is based on the detection of an aptamer tag to a nucleic acid sequence of interest, for example RNA. The aptamer can be detected by a nucleic acid binding protein conjugated to a detector protein. In particular, the nucleic acid binding protein is fragmented and individual fragments are conjugated ti the polypeptide fragments of the detector proteins. Several parameters of the aptamer/protein interaction are important for successful development of nucleic acid-based protein complementation. (i) Binding affinity between RNA aptamer and RNA-binding protein should be high enough to provide energy for the re-assembly of the marker-protein. (ii) The length of the aptamer should not be too long, otherwise introduction of the aptamer into RNA may result in changes in its expression and behavior. (iii) RNA-binding protein should be monomeric, small polypeptide, preferably consisting of two domains that are inactive when separated, but able to bind aptamer, when expressed together.

eIF4A is a eukaryotic initiation factor, and thus is a ubiquitous component of the eukaryotic translation system. Its natural concentration within yeast is high (50 mM) and is comparable to that of another abundant cellular protein, actin (Duncan et al., 1987). It is a 29 kD small protein that acts as an ATP-dependent RNA helicase working in a complex with other initiation factors (4B, 4H, 4G, 4F) (Kapp & Lorsch, 2004). eIF4A consists of two domains and its crystal structure resembles structure of a dumbbell: compact N-terminal and C-terminal domains are connected by a flexible 11 aa-long linker (Johnson & McKay, 1999; Benz et al., 1999; Caruthers et al., 2000). These structural features make this protein a convenient and useful tool for domain dissection. Furthermore, recent studies have isolated strong binding oligonucleotides or aptamers that bind eIF4A with affinity in nanomolar range (Oguro et al., 2003). Oguro et al. showed that a strongly binding aptamer sequence can be as short as 56 nt, and found that dissected domains of eIF4A cannot bind aptamer, while the full-size protein can bind the aptamer with high affinity (Oguro et al., 2003),

Due to these characteristics, eIF4A is a good candidate for protein complementation and was used in our system to drive protein complementation. We used eIF4A which was split into two fragments and fused each of them to fragments of the EGFP detector protein. Re-assembly of eIF4A in the presence of the RNA aptamer therefore brings together the two fragments of the EGFP protein. The affinity of aptamer-eIF4A interaction is in the same range as the MS2 coat protein/MS2 RNA interaction used to monitor RNA within the cell (Bertrand et al., 1998; Beach et al., 1999; Beach & Bloom, 2001; Rook et al., 2000).

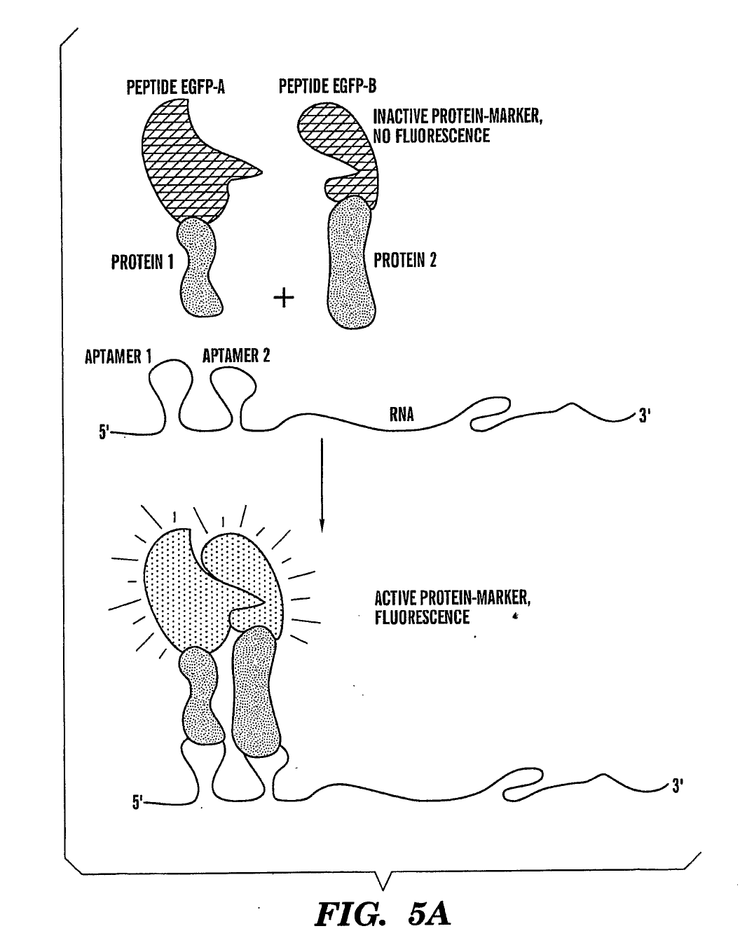

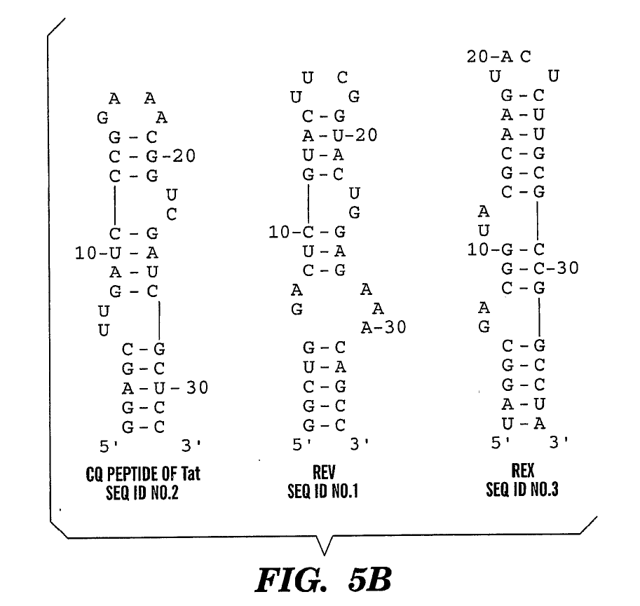

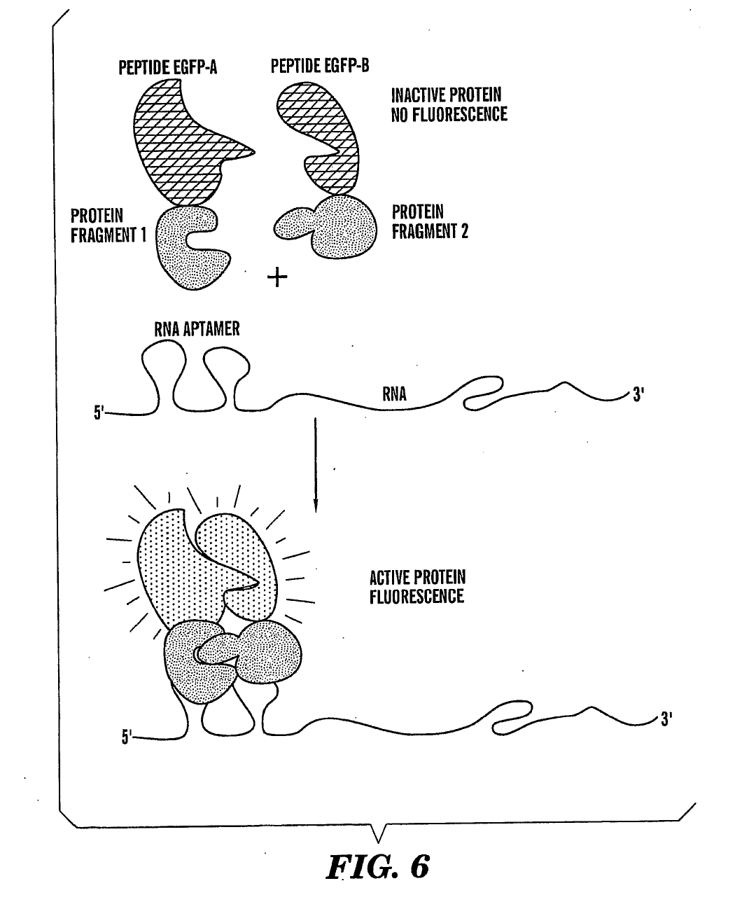

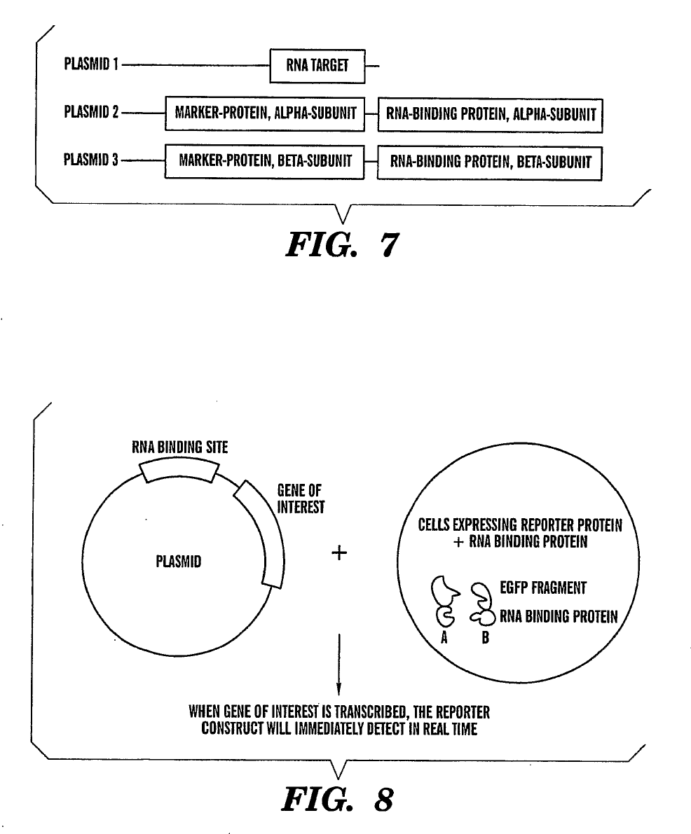

Alternatively, design of the complementing pairs may include two different RNA aptamers and two different proteins or peptides appended to the fragments of the marker protein (FIG. 5). Proteins or peptides in this case will be chosen from a list of viral proteins, for which aptamers with high binding affinity have been isolated in vitro (see Table 1).

TABLE 1
RNA aptamer/peptide pairs with strong affinity binding

| | | | |
|---|---|---|---|
| Peptide | Peptide sequence | Aptamer sequence | Reference |

| | | | |
|---|---|---|---|
| HIV-1 Rex | MPKTRRRPRRSQRKRP | UAGGCGACGGUACGCAAGUA | Baskerville |
| | (SEQ ID NO.4) | CU CUUGCGCCGGCCUA | et al. 1999 |
| | | (SEQ ID NO.3) | |
| Bacterio- | MDAQTRRRERRAEKQA | GGATCCGGGCCCUGAAGAAG | Baron- |
| phage λN | QWKAAN | GGC CCUUUCCUUU | Benhamou |
| | (SEQ ID NO.5) | (SEQ ID NO.8) | et al, 2004 |
| HTLV-1 Rev | TRQARRNRRRRWRERQR | GGCUGGACUCGUACUUCGGU | Ye et al. |
| | (SEQ ID NO.6) | AC UGGAGAAACAGCC | 1996 |
| | | (SEQ ID NO.1) | |
| CQ-peptide | CFLKKGLGISYGRKKRRQ | GGAGCUUGAUCCCGGAAACG | Yamamot et |
| of HIV-1 | RRTAPYDSKNHQDPTPEQ | GUCGAUCGCUCC | al, 2000 |
| Tat protein | (SEQ ID NO.7) | (SEQ ID NO.2) | |

The advantage of this alternative design is that two different proteins or peptides will less likely interact with each other than if one protein is split into two fragments. In case of a split protein, it is still possible that the fragments of dissected protein will spontaneously re-assemble. The major parameter for choosing aptamer/protein pair is stability of the complex (Kd): the stronger the interaction the higher probability of the marker-protein complementation. We reason that similar sizes of the proteins (or peptides) are advantageous for formation of the active complementation complex because of the symmetrical geometry of components.

In vitro selection of artificial aptamers allows finding RNA structures that are highly specific in binding short peptides and bind only the cognate peptide. At the same time, the larger proteins, containing those short peptides, do cross-react with different aptamers (Herman & Patel, 2000). For example, it is known that the Rex protein binds to a portion of the Rev-responsive element and can functionally substitute for Rev (Bogerd et al., 1991; Rimsky et al., 1988); however, in vitro selection lead to isolation of the anti-Rex specific aptamer which does not interact with the Rev peptide (Baskerville et al., 1999). The target RNA can also be designed to contain structural domains for the binding protein. This target RNA will be also expressed from a corresponding plasmid.

Table 1 shows several aptamer and peptide pairs with strong affinity binding that can be used as RNA-tags in protein complementation. Alternatively, additional examples of potential RNA-protein partners, which are to be encompassed in the present invention can be used, selected from a list comprising but not limited to:
(i) MS2 coat protein-RNA stem-loop (Sawata & Taira, 2003; Valegard et al., 1997).
(ii) TAR-Tat BIV-1 stem-loop (Roy et al., 1990; Comolli et al., 1998).
(iii) 3 repeats of the G3/C3 stem-loop shown to work as transcriptional activators (Jarrell & Ptashne, 2003).
(iv) 3 repeats of the aptamer (Yamamoto et al., 2000).
(v) eIF4A—87-nt long aptamer, which can be trimmed to 58 bases, the best Kd=27 nM (Oguro et al., 2003).

### Example 5

### Protein Complementation Directed by Aptamer-Nucleic Acid Binding Protein Interaction

In one embodiment of the present invention, the real time protein-complementation method is based on incorporation of an aptamer-tag into the RNA of interest, which will be recognized by the protein complex consisting of two protein chimeras each containing fragment of the marker-protein and the RNA-binding protein. To reliably use this system, the incorporation of the aptamer-tag should not interfere with RNA synthesis and behavior.

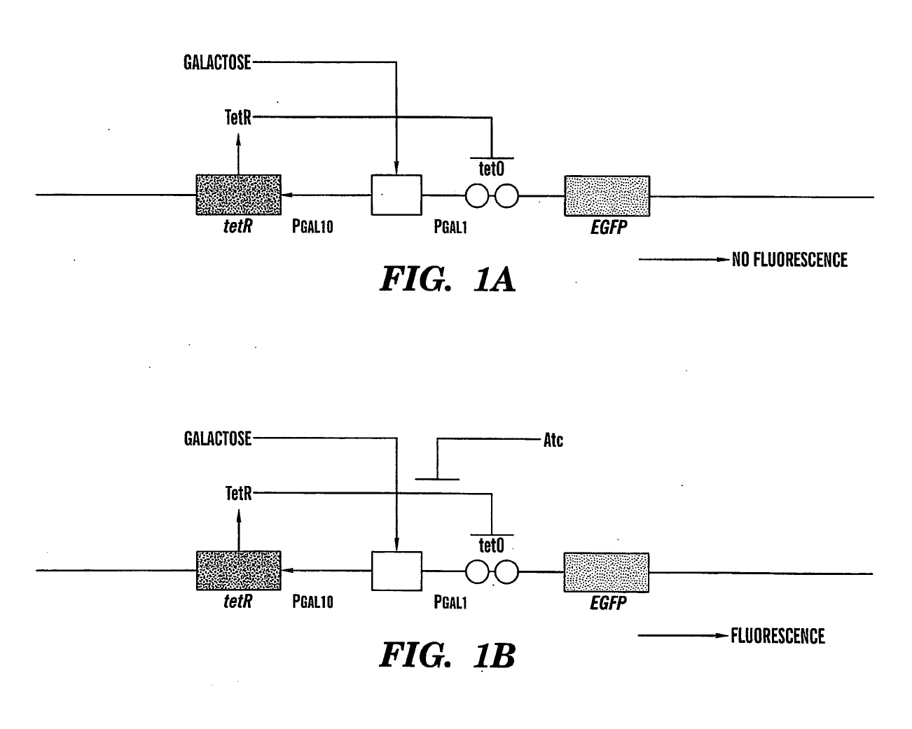

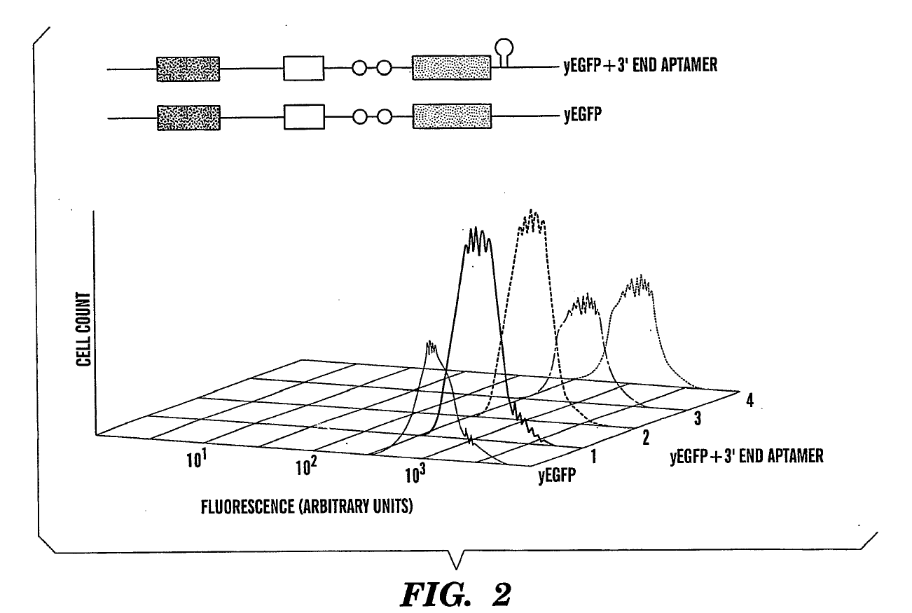

To show the use of protein complementation to monitor RNA of interest, an aptamer was added to a gene of interest. To show the aptamer did not affect eukaryotic gene expression, we introduced the 64-nt oligomer (Oguro et al., 2003) right after the stop codon of a reporter gene in Saccharomyces cerevisiae strain YPH500 (MATα, ura3-52, lys2-801amber, ade2-101ochre trp1-Δ63, his3-Δ200, leu2-Δ1). We used an inducible system where the reporter gene, e.g. EGFP, is under an artificial Tet-responsive GAL1 promoter (Blake et al., 2003). In this system, a TetR gene is expressed from the GAL10 promoter so that in the presence of galactose, TetR mediates the repression of EGFP (FIG. 1A). This repression can be relieved by the addition of the inducer anhydrotetracycline (ATc), which binds directly to TetR (FIG. 1B). Expression of EGFP in the presence of galactose and ATc was then quantified by flow cytometric analysis (FACS) in cells containing a chromosomal integration of the inducible transcriptional system. We observed no difference in fluorescence between cells expressing the unmodified EGFP and cells where aptamer was inserted at the 3′ end of this gene (FIG. 2). Thus, 3′-tagging of a gene with an aptamer sequence does not seem to affect gene expression at the protein level. We reasonably assumed that RNA level is also unaffected by the presence of this aptamer-tag.

Analysis of literature shows that aptamer sequences can be incorporated within 5′- or 3′-untranslated RNA regions without interfering with RNA synthesis (Hanson et al., 2003; Nickens et al., 2003), while it can affect efficiency of translation, especially if the incorporated aptamer is interacting with some translational regulator, e.g., tetracycline (Hanson et al., 2003). Our preliminary results corroborate these data and show that incorporation of the aptamer against eIF4A into 3′-untranslated region does not affect expression of the marker-protein EGFP in yeast, which indicates that EGFP mRNA level was also unaffected.

To make the system more flexible, one can expand these experiments by testing whether 5′-untranslated region can also be used for aptamer incorporation. For example, using EGFP as a marker protein, and its expression will be measured in vivo depending on the position of the aptamer-tag. The impact of the aptamer will be followed by measuring total cell fluorescence using a fluorescent cell sorter (FACS) and by Northern blot analysis, which will directly estimate RNA transcription level. Still, the most likely site for incorporation of the aptamer-tag is the 3′-untranslated region.

The impact of tag incorporation on RNA movement and localization is also a consideration. The 3′-UTRs of mRNAs contain regulatory elements determining mRNA localization (for review, see Hesketh, 2004). For example, 3′ UTR of the ASH1 mRNA encodes signals sufficient for mRNA transport and anchorage within the bud. However, in many cases sequences within other parts of the message are also necessary for proper RNA localization (Chartrand et al., 1999, Gonzalez et al., 1999). For example, the shortest possible aptamer-tag is used without removing any parts of the message. In doing so, all regulatory RNA sequences are maintained to preserve all RNA/protein interactions and result in intact localization of RNA. Positioning of the re-assembled protein complex on the RNA template is quite tolerable given small dimensions of fluorescent proteins (or beta-lactamase) and small peptides interacting with aptamers within RNA. Nevertheless, proper localization of the modified RNA are directly checked in control experiments by in situ hybridization with the probes specific for the given mRNA.

### Example 6

### Development of a System for Equimolar Co-Expression of the Two Chimeric Proteins Each Containing a Fragment of the Fluorescent Marker-Protein and of the RNA-Binding Protein

For successful real-time protein complementation method of this invention the coordinated synthesis of the two polypeptide protein fragments, each fragment comprising the activated marker-protein conjugated to a RNA-binding protein and produced in equimolar amounts and ready to re-associate. Previous studies of protein complementation showed that if the two protein fusions were synthesized from plasmids with different copy number, no fluorescent recovery of GFP was detected (Magliery et al., 2005). Thus, we developed a system in which equimolar synthesis of protein fragments occurs and an inducible system for the independent synthesis of the RNA component.

To express two chimeric proteins in equimolar amounts we used expression vectors allowing co-expression of several messages (pETDuet or pACYCDuet vectors, Novagen). These plasmids have two T7 promoters and two multiple cloning sites, therefore each plasmid can support expression of two messages. At the same time origins of replication in these plasmids are different allowing their co-expression. We placed two chimeric proteins in one plasmid and RNA with a tag in a different plasmid; so that RNA expression can be induced independently. pBAD plasmid for independent induction of RNA expression will be used. The expression level of chimeric proteins is tested by Western blot analysis with anti-EGFP antibodies (Clontech), while RNA synthesis is tested by Northern blotting.

These experiments are performed in E. coli and protein complementation is monitored by measuring total cell fluorescence using FACS. As controls, the background of the cells expressing two protein chimeras in the absence of the RNA component did not show fluorescence (FIG. 9A), whereas their expression in the presence of aptamers resulted in fluorescence (FIG. 9C) verifies the specificity of the aptamer/RNA-binding protein/peptide interaction.

### Example 7

### Use of Beta-Lactamase as a Split-Polypeptide Detector Protein

The class A beta-lactamases have been used as split marker-proteins in protein complementation assay by two independent groups (Wehrman et al., 2002; Galarneau et al., 2002), which can be explained by several attractive features of this class of enzymes. First, these proteins are relatively small monomeric enzymes, and their crystal structure is well known (Jelsch et al., 1993). beta-lactamase can be expressed both in bacterial and eukaryotic cells; importantly eukaryotic cells do not have endogenous lactamase activity. Another significant factor that makes using beta-lactamases in vivo a powerful tool is the availability of cell-permeable fluorescent substrate developed by the Tsien group (Zlokarnik et al., 1997).

It has been shown by both groups mentioned above that beta-lactamase can be dissected at residues 196-198, and that these fragments can complement one another in the presence of the appended mutually interacting proteins (Wehrman et al., 2002; Galameau et al., 2002). This site (196-198 amino acids) is located on the opposite site from the catalytic center and does not show periodic secondary structure. Blau's group show that incorporation of the Asp-Gly-Arg tripeptide into the C-terminus of the N-terminal fragment of beta-lactamase increased activity of the enzyme up to 10,000-fold (Wehrman et al., 2002). As a result, protein complementation based on beta-lactamase activity allows signal amplification about two orders of magnitude and the signal can be detected within minutes after induction of the protein (Wehrman et al., 2002).

It should be emphasized that protein complementation based on reconstitution of the enzymatic activity is promising in terms of sensitivity; however there may be a loss in spatial resolution because of the diffusion of the signal. Thus, this format of the assay should preferably be applied in cases when spatial resolution is not as important and where, for example, detection of RNA's of low abundance is important.

In Methods using β-lactamase as the detector protein for in vivo RNA detection assay in prokaryotic and eukaryotic cells, beta-lactamase can be split into activated polypeptide fragments as shown by Blau's group (Wehrman et al., 2002). Two fragments, one consisting of amino acid residues 24 to 197 (alpha-fragment lacking periplasmic secretory signal system to keep the enzyme within the cell) and the second one consisting of residues 198 to 240 (beta-fragment) are cloned in E. coli. The alpha- and of beta-fragments will be first PCR amplified from pUC19, and the tripeptide NGR will be added to the C-terminus of the alpha-fragment by PCR. PCR product coding for the alpha-fragment will be cloned downstream of the F1 fragment of eIF4A with a polypeptide linker, while the beta-fragment of beta-lactamase will be cloned downstream of the F2 fragment of the eIF4A.

For prokaryotic expression, the chimeric split β-lactamase-eIF4A conjugated proteins are introduced into pCDF-duet vector (Novagen), while the RNA target with the eIF4A binding aptamer will be expressed from a pACYCD plasmid. The resulting plasmids will be co-expressed in E. coli strain BL21 (DE3) (Novagen).

The expression of beta-lactamase conjugated to eIF4A polypeptide fragments in eukaryotic cells will be done in Saccharomyces cerevisiae. Plasmids used to express chimeric proteins containing fragments of beta-lactamase and eIF4A [beta-lactamase(A)-(F1) and beta-lactamase(B)-(F2)] in yeast will be derivatives of the expression vector pDB20 (Becker et al., 1991) modified to express proteins fused to an HA or myc epitope tag at their N-termini. This will allow constitutive protein expression from the strong ADH1 promoter and analysis of gene expression by Western blots. Plasmid pRS4D1 (Blake et al. 2003) will be modified to express the target RNA (in place of EGFP) with the 64 nt-long aptamer tag placed at the 3′ end of the gene. Integration of this plasmid into the genome of strain YPH500 will allow the regulated expression of the tagged RNA upon addition of galactose and anhydrotetracycline to the culture medium (Blale et al. 2003).

When no beta-lactamase activity is present, excitation of the cumarin at 409 nm will lead to FRET and emission at 520 will take place (green fluorescence). beta-lactamase activity occurs due to the interaction between the aptamer and split-eIF4A fragments, it will open the beta-lactam ring and fluorescein will split off, no FRET will take place in this case and emission at 447 nm will be observed (blue fluorescence) (Zlokarnik et al. 1998).

### Example 8

### Detection of RNA Molecules by Fast Protein Complementation In Vitro and In Vivo

In the following example we developed a robust method of RNA detection within the living cell that has sensitivity exceeding current levels of detection. The combination of real time fast kinetic protein complementation using activated split-fluorescent fragments (see example 2), which allows substantially quick reconstitution of the active detector protein and reduced background, together with signal amplification, introduced by the use of enzymatic step, results in a detection technique capable of analyzing nucleic acids of moderate abundance.

A protein with enzymatic activity or fluorogenic properties is split into two parts (termed α- and β-subunits). These parts are expressed in vivo as chimeras with a nucleic acid binding protein which has an aptamer-binding domain. An ideal nucleic acid-binding protein would consist of two parts, which are inactive by itself but are active together. In the presence of an RNA, which contains a motif recognizable by the nucleic acid-binding protein, the activity of the detector protein is immediately restored. In case the protein detector is an enzyme, the signal is amplified. In case the protein is fluorogenic, there is no signal amplification, but there is also no background, because fluorescence is completely dependent on the presence of target RNA.

We made constructs for the co-expression of protein fusions and aptamer-containing RNA transcripts in E. coli BL21 (DE)₃ cells (FIG. 9). We fused the C-terminus of EGFP fragment (residues 1-158) to the N-terminus of eIF4A fragment containing residues 1-215 via a flexible polypeptide linker consisting of Serine and Glycine residues. We cloned this fusion in the first multiple cloning site (MCS) of vector pACYCDuet-1 (Novagen) designed for the expression of two open reading frames from two T7 promoters. Similarly, we fused the C-terminus of EGFP fragment (residues 159-238) to the N-terminus of eIF4A fragment containing amino acids 216-406 via a flexible polypeptide linker. This fusion was cloned in the second MCS of the vector pACYCDuet-1 to create a construct able to express the two fusion proteins in approximately equimolar amounts. The vector, pETDuet-1 (Novagen) was used for the expression of a 360 nt-long T7-transcript containing two copies of the eIF4A-interacting aptamer sequence in tandem. This small message consisted of a 33-nt leader sequence, followed by two copies of aptamer sequence, and about 200 nt of the nuclease-resistant T7 termination sequence.

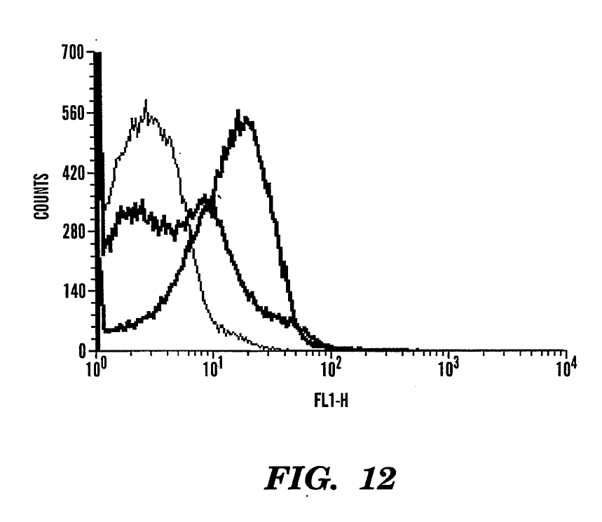

E. coli cells expressing the entire complementation complex and appropriate controls were grown at room temperature in the presence of the inducer, isopropyl-β-D-thiogalactopyranoside (IPTG) for co-expression of proteins and RNA. When cultures reached an optical density of approximately 0.5 (OD₆₀₀=0.5), they were analyzed by fluorescence activated cell sorting (FACS) (FIG. 21). Co-expression of the complementary fusion proteins along with the aptamer-containing RNA transcript resulted in a 10-20 fold increase in average fluorescence (FIG. 9C). However, in the absence of aptamer-containing transcript, E. coli cells bearing the complementary fusion proteins did not display fluorescence above background levels (FIG. 9A). More importantly, we did not see a significant increase in fluorescence when cells were expressing the protein components of the complementing complex along with an untagged transcript (FIG. 12). There was no difference in fluorescence yield when the T7 transcript had or lacked ribosome binding site. This suggests that translation of a message does not interfere with its detection.

### Example 9

### Quantification of RNA Molecules In Vivo

Fluorescence of cells expressing intact EGFP was about 30-50 fold higher than that of cells with the complementation system (FIGS. 9B and 9C). This difference is not surprising since there should be a greater amount of full-length EGFP compared to the re-assembled EGFP, whose concentration is determined by RNA concentration. It is known that each RNA molecule is recycled by ribosomes several times, which results in a molar ratio of protein to RNA larger than one.

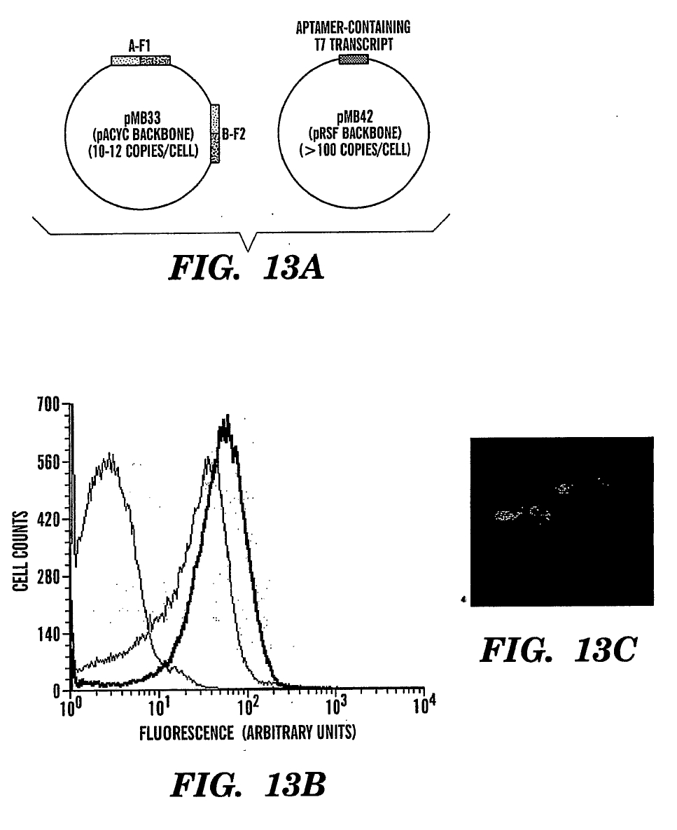

We next made use of EGFP calibration beads (BD Biosciences Clontech) to evaluate the absolute average number of the re-assembled EGFP molecules per cell and thus the efficiency of our detection system. We found that cells expressing RNA from a pET vector with a copy number of about 40, produced 500-600 molecules of reassembled EGFP which corresponds to 1-2 μM RNA, if the cell volume is 1.41×10⁻¹⁵ L (Lee, et al, 2005). This RNA concentration correlates well with the experimental results obtained in E. coli from a vector with 50-70 copies/cell (Lee, et al, 2005). The concordance between expected and experimental results suggests that practically all molecules of aptamer-containing RNA are detected by the re-assembled protein complex. We tested this assumption by increasing the copy number of the plasmid bearing the RNA aptamer (from 40-100 copies/cell) and observed that bacterial cells displayed higher fluorescence when compared to the original construct (FIG. 13). This result supports our conclusion that nearly all RNA molecules are detected by our complementation system.

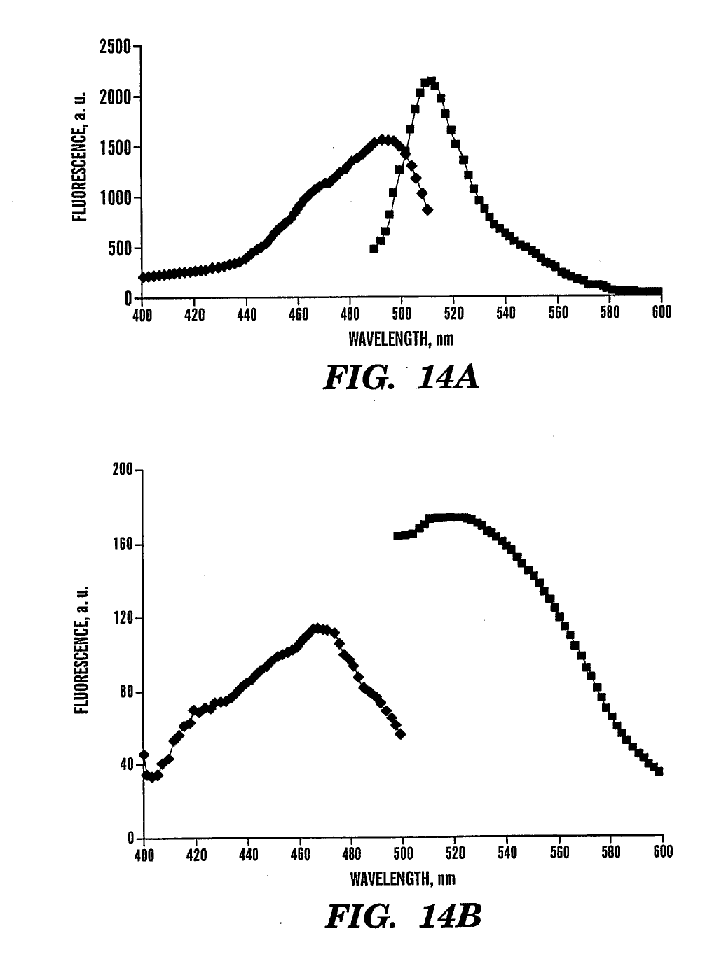

We then compared the fluorescence spectra of cells bearing the re-assembled EGFP with that of cells expressing the full-length protein. We found in these experiments that a maximum excitation at 470 nm for the re-assembled complex (versus 490 nm for native EGFP), and a maximum emission about 520 μm (versus 508 nm for the native EGFP) (FIG. 14). This difference can be explained by possible changes in protein conformation between the native EGFP and the re-assembled EGFP-RNA complex. This result also supports the notion that fluorescent signal within the cell is due to the formation of a nucleoprotein complex, since in our in vitro experiments we also found a similar red-shifted emission spectrum (max 524 nm) for split EGFP re-assembled by appended duplex oligonucleotides (Demidov et al, 2006).

### Example 10

### Spatial and Temporal Oscillations of Fluorescence in Bacterial Cells

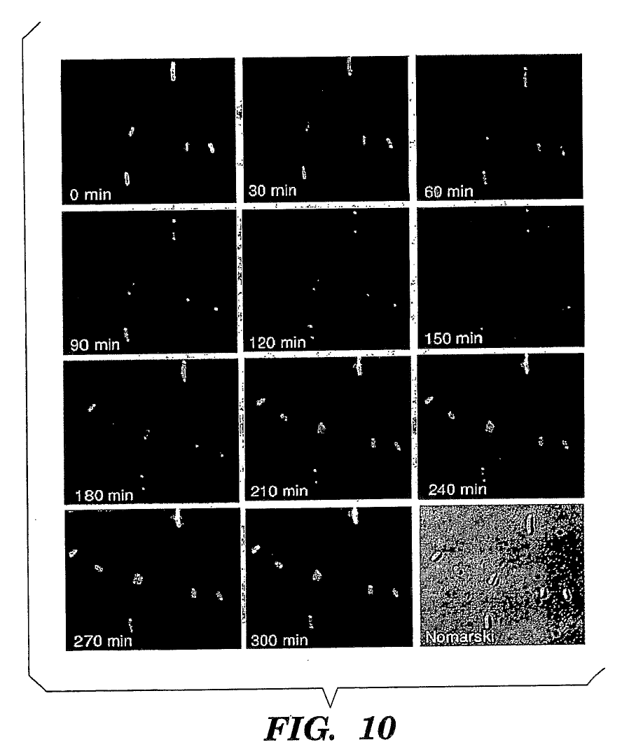

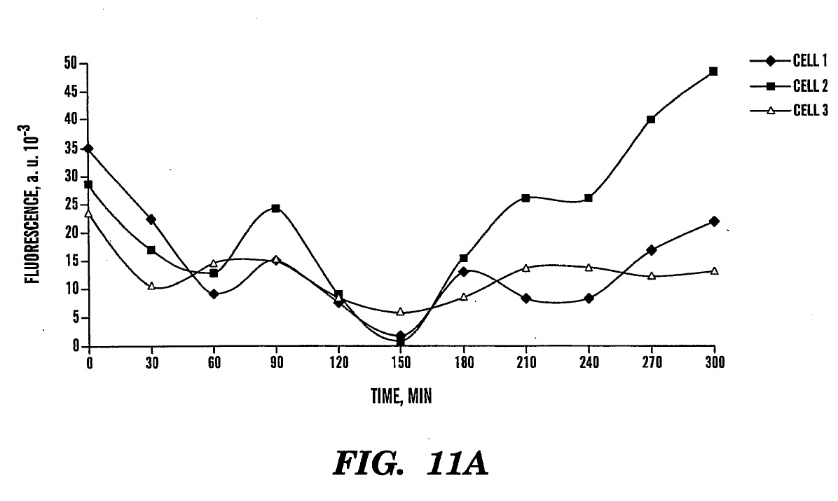

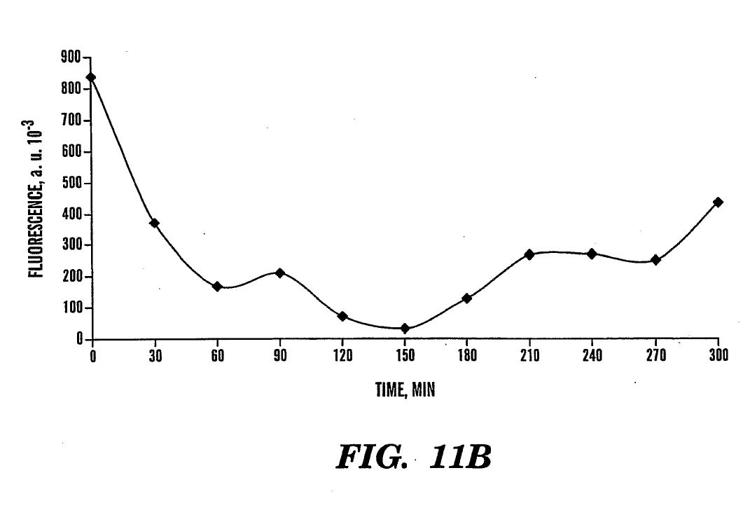

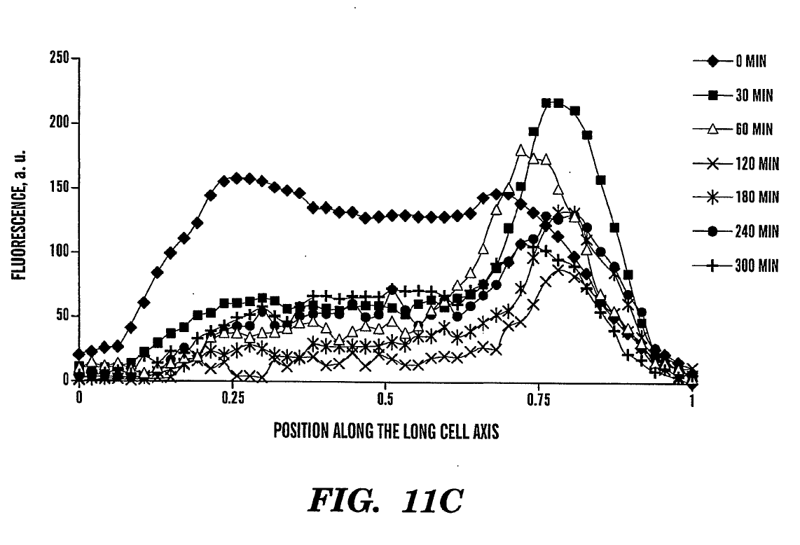

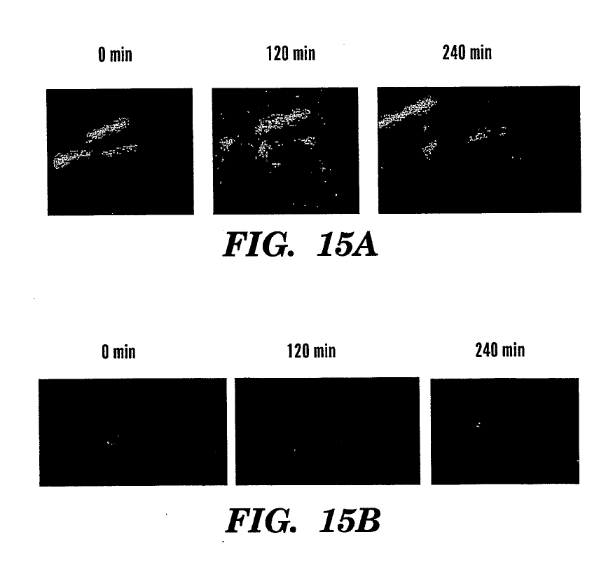

An epifluorescence microscope and B&W camera were used to observe the cells expressing the RNA labeling system (FIGS. 9c and 11). In parallel, differential interference contrast images were recorded to analyze cell numbers and shapes. To allow enough time for complex formation and fluorescence development, cells were grown overnight at room temperature Therefore, the majority of cells were in stationary phase when little or no division occurs. In some instances, we did observe cell division, and in all cases the newly divided cells were not fluorescent (FIG. 15).

Co-expression of the protein fusions and RNA transcript containing aptamer resulted in fluorescent cells with bright fluorescent spots located at one or both poles of the cell (FIG. 10). In contrast, expression of the full-size EGFP produced strongly fluorescent cells with a uniform distribution of fluorescence throughout the cell (FIG. 9b). At the same time, expression of protein fusions in the absence of the aptamer expression did not lead to significant fluorescence development (FIG. 9a), which confirms our previous flow cytometry results.

Time-lapse microscopy revealed several remarkable features of the fluorescent particles as well as changes in total fluorescence of the cells. FIG. 10 shows a sequence of images taken with 30 min intervals in one representative experiment. Cells in the same field showed synchronous oscillations of different amplitude (FIG. 11c). Total fluorescence in each cell gradually dropped during the first two hours, but later increased again. Simultaneously, a decrease in total cell fluorescence resulted in the appearance of the high-fluorescence particles at the cell poles. Interestingly, several cells became fluorescent during the course of the experiment (FIG. 11b, 180 min and thereafter). The kinetics of these changes varied from experiment to experiment, but the overall pattern of increase and decrease of fluorescence over time was always the same.

To show that changes in cell fluorescence actually reveal RNA dynamics we extracted total RNA from cell culture and used real competitive PCR (rcPCR) coupled with MALDI-TOF MS detection to determine the absolute concentration of the aptamer and mreB RNA, a housekeeping gene used for normalization. Real competitive PCR utilizes a serially diluted DNA competitor to act as an internal standard for the gene of interest prior to amplification. A single base difference between the competitor and the gene of interest is exploited in a 1 or 2 nt base extension reaction using MALDI-TOF MS to quantify the abundance of the extension products.

Analysis of the aptamer mRNA levels shows statistically significant oscillations while the control gene, mreB, remains constant (Table 2). The data show that at the 1 hour mark the aptamer transcript reaches a peak then promptly decreases back to the time-zero baseline at 120 to 150 minutes. This is followed by another transcript peak at 170 minutes and finally an abrupt drop and then recovery back to initial time-zero aptamer mRNA levels.

TABLE 2
Aptamer mRNA transcript levels and TITAN
(Elvidge et al, 2006) statistics

| | |
|---|---|
| Time | Equivilance Point (M) |

| (Min) | mreB | Aptamer | Fold | P-value | R² |
|---|---|---|---|---|---|
| 0 | 2.67E−14 | 4.52E−11 | 1.00 | — | 0.975 |
| 60 | 2.45E−14 | 5.49E−11 | 1.32 | 0.033 | 0.986 |
| 120 | 2.57E−14 | 4.76E−11 | 1.09 | 0.260 | 0.990 |
| 150 | 2.42E−14 | 4.47E−11 | 1.09 | 0.243 | 0.969 |
| 170 | 2.54E−14 | 5.33E−11 | 1.24 | 0.052 | 0.989 |
| 210 | 2.69E−14 | 3.73E−11 | 0.82 | 0.081 | 0.988 |
| 240 | 2.06E−14 | 3.51E−11 | 1.01 | 0.496 | 0.987 |
| 270 | 2.48E−14 | 4.14E−11 | 0.98 | 0.452 | 0.984 |

Concentration values are for the sample of RNA extracted from each cell culture plate. Fold values adjusted for housekeeping control gene mreB and calculated relative to the time-zero baseline. The bootstrap P value is a measure of the confidence that the gene is differently expressed from baseline. All analyzed samples are <0.05 for residual and lack-of-fit values; indications of a good quality assay.

Application of the techniques used in the Example 9 and this example can be used to detect RNA localization and movement. One example of such an application of this new technology one can study ASH1 mRNA. This RNA codes for a cell-fate determinant that inhibits transcription of HO endonuclease and blocks mating-type interconversion in the daughter-cell. Its localization to the bud tip of the budded yeast cells has been shown by different methods. ASH1 mRNA is well studied; therefore it can be used as a model experiment to verify sensitivity and specificity of the analysis of RNA localization and movement in living cells.

### Example 11

### Detection of RNA In Vivo Using Binary Aptamer-Peptide Interactions

In experiments 9 and 10 we showed monitoring of RNA in vivo, which uses combination of fluorescent protein complementation and high affinity interaction of the RNA-binding protein with the RNA-aptamer. In these experiments, the RNA-binding protein is the eukaryotic initiation factor 4A (eIF4A) that consists of a dumbbell-shaped structure. eIF4A is dissected into two fragments, and each fragment is fused to split fragments of the enhanced green fluorescent protein (EGFP). Co-expression of the two complementing protein fusions and of a transcript containing eIF4A-specific aptamer resulted in the restoration of EGFP fluorescence in bacteria. The major advantage of this approach is that the fluorescent signal is solely determined by the target RNA, in other words in the absence of RNA there is no detectable signal. Additionally, a relatively small protein complex is assembled on the target RNA, which should not interfere with RNA function and localization.

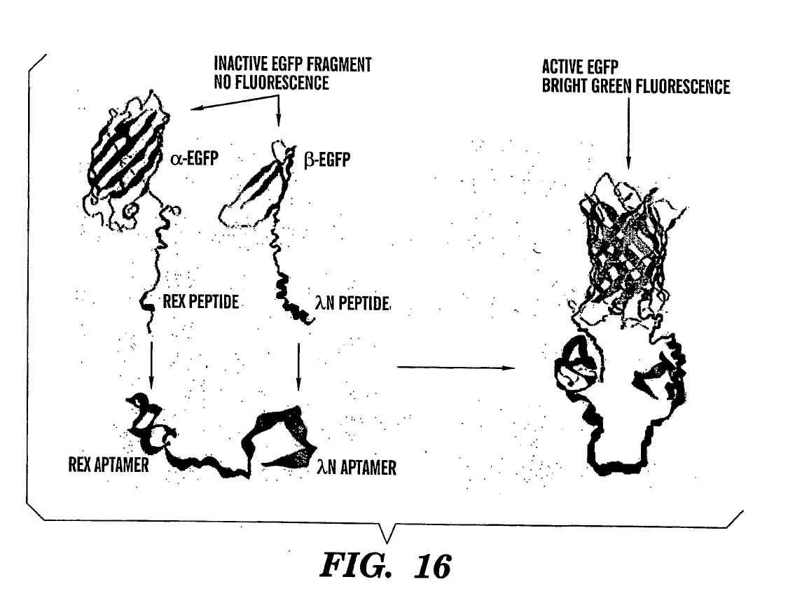

In experiment 11, we present the data on development of an alternative embodiment for RNA visualization, where the aptamer-binding moieties are two short peptides. In this approach two different RNA aptamers are added as tags to RNA of interest and are recognized by two short viral peptides fused with the fragments of a split EGFP (FIG. 16). There are several reasons for these modifications. First, the protein complex which is assembled on the target RNA is substantially smaller as compared with that containing re-assembled eIF4A. In general, the smaller the detection tool, the lower the probability of interference with function. Second, eIF4A is a component of the translational machinery in eukaryotic cells and it also has close homologs among bacterial proteins. Therefore, there is some probability that its over-expression may interfere with normal cell functioning. At the same time, short viral peptides do not have homologous proteins in bacterial or eukaryotic cells and therefore their expression in the cell is more likely to be neutral to the cell functions. Finally, an alternative design of the RNA recognizing complex adds more flexibility to the new approach and shows its universality.

Design of the complementation complex for RNA detection in vivo based on binary aptamer-peptide interactions. According to our scheme, for detecting RNA in the live cell, RNA should be supplied with two aptamer sequences capable of interaction with the two peptides. Each peptide should be expressed in the cell as a fusion with one of the two fragments of a split enhanced green fluorescent protein (EGFP) (FIG. 16). In order to make such a system work, several issues were considered; first, the affinity of each interacting peptide/aptamer pair selected should be high and bind with comparable affinity for both pairs. Second, there should be no cross-reactivity between the two aptamer/peptide pairs, in other words specificity of each interaction should be high enough. Third, there should be no interaction between the peptides, which could otherwise bring fragments of the marker-protein together and thus increase the non-specific background. Next, the length of the peptides should be of comparable length to allow symmetrical assembly of the complementing complex. Finally, the placing of the two aptamers should allow independent interaction of each of them with the corresponding peptide. This can be accomplished using a flexible linker placed between the aptamers.

Peptides with arginine-rich motifs (ARM) are the fragments that determine high affinity and high specificity of interaction of many RNA-binding proteins with corresponding RNA targets (22). The common feature of these peptides is a preponderance of arginines in the absence of other similarity. Recent studies aimed at understanding the mechanism of ARM-peptides interaction with the corresponding RNAs concluded that specific pattern of arginine position and flexibility of the peptide backbone are responsible for the specific binding of a corresponding RNA ligand (23). However, many ARM-peptides display ‘chameleon-like’ behavior and bind many RNA targets, although with lesser affinity (24). Keeping this in mind, we chose RNA-binding peptides from the ARM viral peptides (25-27), but tested their cross-reactivity with the corresponding RNAs before applying them in protein complementation.

Experiments in vitro to choose aptamer-peptide pairs. Based on available data we chose three peptide/aptamer pairs; (i) HIV Rex; (ii) Bacterophage λN; and (iii) HTLV-1 Rev (see Table 1) and tested their binding affinity with cognate partner and cross-reactivity with two other RNA aptamers in vitro. We used non-radioactive gel-shift assay under conditions when fixed concentrations of RNA aptamer were allowed to form complexes with increasing ARM-peptide concentrations. We quantified the amount of the unbound RNA aptamer and the amount of the shifted complex (Table 1). The results showed that the λN peptide and HIV-1 Rex peptide displayed high specificity and did not cross-react with the non-matched RNA aptamers. At the same time, HTLV-1 Rev peptide did show some cross-reactivity with the two non-matched RNA aptamers. Based on these results, we concluded that the λN and Rex peptide of HTLV-1 with their corresponding aptamers provided the optimal pairs for using in our protein complementation complex (see Table 3).

TABLE 3
Cross-reactivity within three RNA aptamer/peptide pairs

| | Aptamer | | |
|---|---|---|---|
| Peptide | HIV-1 Rex apt | λN apt | HTLV-1 Rev |
| HIV-1 Rex peptide | + | − | − |
| Bacteriophage λN peptide | − | + | − |
| HTLV-1 Rev peptide | + | + | + |

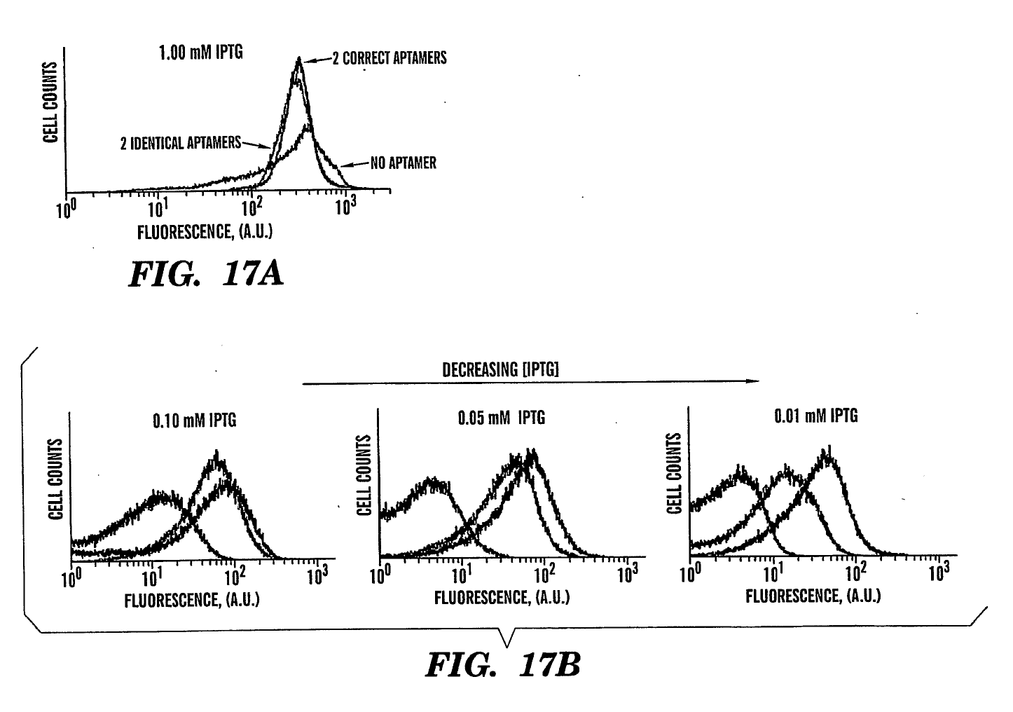

Detection of RNA transcripts in live bacterial cells using binary peptide/aptamer interactions. Protein fusions and aptamer-containing RNA transcripts were cloned and expressed in E. coli BL21 (DE)3 cells (FIG. 17). The C-terminus of EGFP fragment (residues 1-158) was fused to the N-terminus of the Rex peptide (16 aa) via a flexible linker consisting of serine and glycine residues. Similarly, the N-terminus of the second EGFP fragment (residues 159-238) was fused to the C-terminus of % N peptide (22 aa) also via a polypeptide linker. The entire DNA insert coding for both protein fusions and the T7 promoter region in between was synthesized by multi-step PCR and cloned into the vector pACYCDuet-1 between the NcoI and AvrII sites to create a construct able to co-express two fusion proteins. The vector, pETDuet-1 (Novagen) was used for the expression of a 230 nt-long T7-transcript containing two aptamer sequences linked by 5 or 10 dT residues. This message consisted of a leader sequence, followed by two aptamer sequences, and about 200 nt of the nuclease-resistant T7 termination sequence.

E. coli cells expressing the entire complementation complex and appropriate controls were grown overnight at room temperature in the presence of the inducer, isopropyl-β-D-thiogalactopyranoside (IPTG) for co-expression of proteins and RNA. When cultures reached an optical density of approximately 0.5 (OD₆₀₀=0.5), they were analyzed by fluorescence activated cell sorting (FACS) (FIG. 17). Fluorescence of E. coli cells expressing the entire complementation complex was compared with fluorescence of the cells expressing two protein fusions only and two protein fusions plus an incorrect combination of the two aptamers (two identical Rex peptide-binding aptamers linked with 10 dT nucleotides).

The results showed that induction with 1 mM IPTG resulted in high cell fluorescence in the absence of RNA expression (FIG. 17A), and there also was no difference in fluorescence distribution of the cells expressing correct and incorrect aptamer sequences (FIG. 17A). We suggested that T7 promoters induced with 1 mM IPTG expressed too high concentration of the protein fusions, which re-assembled even in the absence of the RNA target. If this suggestion was correct, reducing IPTG concentration would lead to reduced background. Indeed, decreasing concentration of IPTG 10-fold resulted in separation of the fluorescence distributions for the cells expressing protein fusions only and those expressing proteins with two correct RNA aptamers. However, specificity was still not high enough to discriminate correct versus incorrect aptamer sequences. Finally, decreasing concentration of IPTG to 0.01 mM allowed separating fluorescence distributions for the cells with the correct and incorrect aptamer sequences. Under these optimized conditions, average fluorescence of the cells expressing the entire complementation complex exceeded background fluorescence (no RNA component) 10-15 fold, and the cells with correct aptamer sequences displayed 4-5 times higher fluorescence than the cells with the incorrect ones.

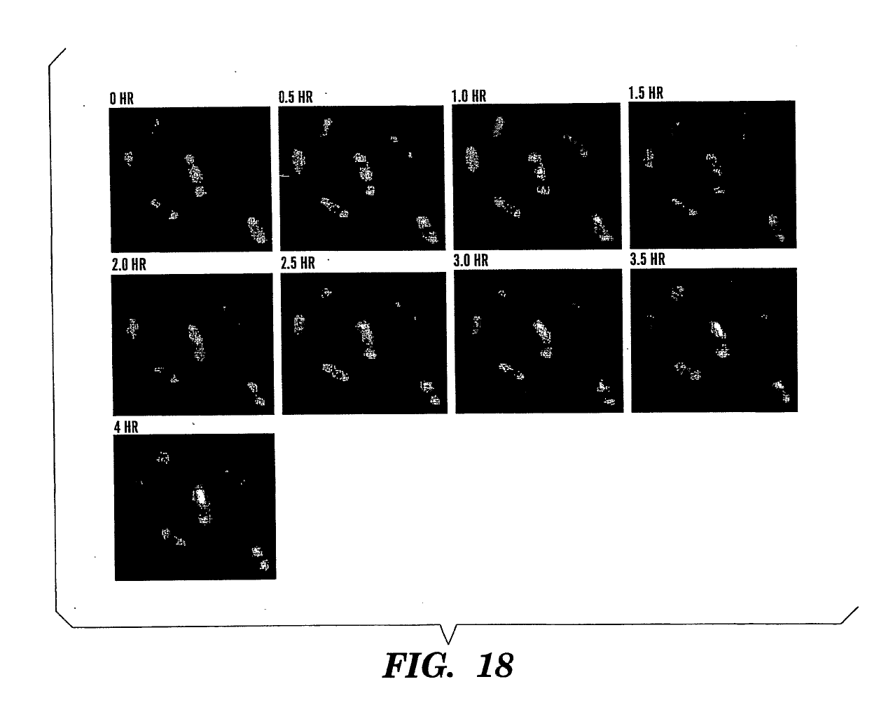

Dynamics of fluorescence changes. Bacterial cells expressing the entire complementation complex in optimized conditions were analyzed using fluorescent microscopy. Different types of fluorescence distribution were observed (FIG. 18). In most cells fluorescence was highest at the poles similar to the results obtained in the experiments when protein complementation was triggered by eIF4A-aptamer interactions. Some cells (about 10%) had fluorescent particles localized in the middle of the cell analogous to the results reported by Golding and Cox (18).

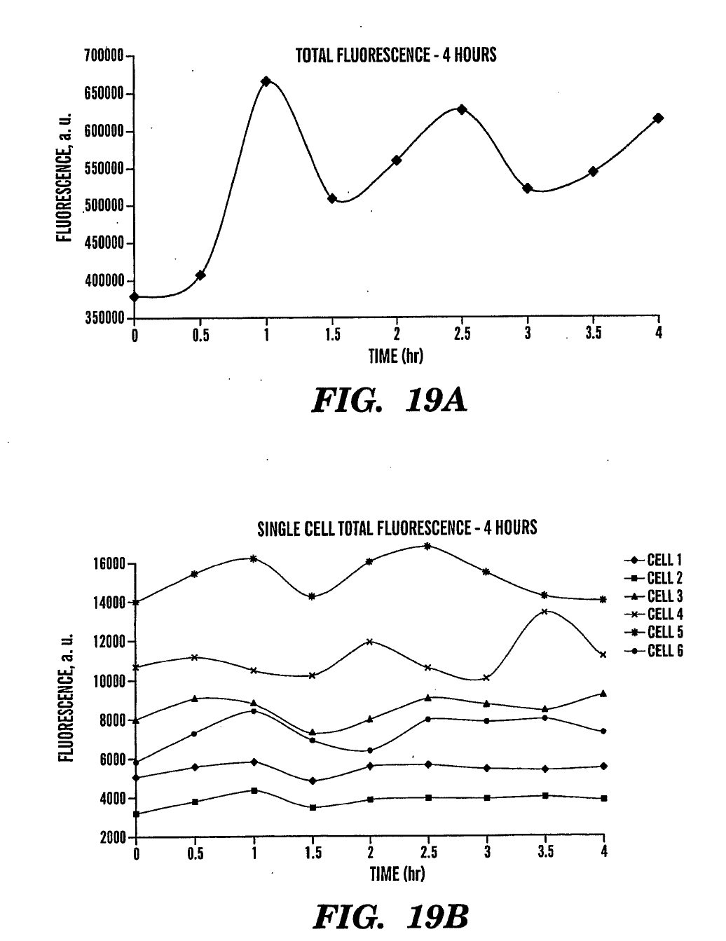

Time-lapse imaging revealed temporal and spatial changes in fluorescence which were also similar to the results with eIF4A-based complementation system. FIG. 18 shows a sequence of images taken with 30 min intervals for 4 hours in one representative experiment. Cell fluorescence showed oscillatory changes and oscillations were synchronous in different cells (FIG. 19b).

The results obtained with the binary aptamer-peptide interactions and protein complementation system are very similar in several respects to those obtained with the system using split initiation factor 4A (eIF4A) interactions with the corresponding aptamer. First, the localization of fluorescent particles in vast majority of the cells at the poles is characteristic for both methods. Second, the dynamics of fluorescence changes in time and in cellular space is also similar to that in eIF4A-based system. Finally, synchronization in fluorescence changes between the cells is visible in both systems. These results imply that the nature of the RNA-protein binding protein or peptide used in the protein complementation complex is not important for the system to work and other split proteins or short peptides can be used in similar applications. The published results on using MS2 coat protein-based system for RNA visualization in live cells also supports this conclusion (18, 20).

It is interesting to note at the same time, comparison of two complementation designs revealed differences in the strength of fluorescent signal and signal/background ratio. For reasons, which we do not fully understand, fluorescence of the cells expressing short peptides fused to split EGFP was much higher than fluorescence of the cells expressing fragments of eIF4A fused with EGFP. We assume that the ARM-peptides due to their positive charges and their capacity to interact with negatively charged proteins and DNA are more prone to association through the third party molecules than neutral eIF4A fragments. This results in EGFP reassembly and higher background. In attempt to reduce the background we reduced concentration of IPTG and found conditions under which the signal/background ratio was 10-20 fold, which is in the same range as in the case of eIF4A system. Moreover, discrimination between the correct and wrong aptamer sequences was also achieved under these conditions.

Materials and Methods.

Constructs and Strains.

pMB33 and pMB38 are derivatives of pACYCDuet-1 (Novagen) into which the interacting protein fragments and the ORF of EGFP were cloned, respectively. Cloning of eIF4A fragments 1 (F1: 1-215 aa) into pACYCDuet-1 (pMB09); and 2 (F2: 216-406 aa) into pETDuet-1 (Novagen) (pMB11) was done by PCR-amplification of the mouse eIF4A protein from plasmid pGEX-4AI (kindly donated by Dr. Chris Proud). Similarly, EGFP fragments, Alpha (A: 1-158 aa) and Beta (B: 159-238 aa) were PCR-amplified from pEGFP (Clonetech) and cloned into pACYCDuet-1 (pMB08) and pETDuet-1 (pMB10), respectively. A chimeric gene (pMB12) where the C-terminal end of EGFP fragment A is fused to the N-terminus of eIF4A fragment F1 via a 10-aa flexible polypeptide linker (Gly-Ser-Ser-Gly-Ser-Ser-Gly-Ser-Gly-Ser) was generated according to Vasl et al., 2004. (See Supplementary methods for details). A similar protocol was used to create the fusion B-F2 (pMB13). Cloning of B-F2 into pMB12, a derivative of pACYCDuet-1 (pMB33), was done as described previously (Geiser et al., 2001). pMB23 expressing a T7-transcript containing the eIF4A-interacting aptamer sequence (58 nt-long) was a derivative of pETDuet-1 (Novagen). pMB42 was a derivative of pRSFDuet (Novagen) that also expressed the sequence for the eIF4A-interacting aptamer (See Supplementary methods for more details). E. coli strains XL10-Gold (Tet^{r} Δ(mcrA)183 Δ(mcrCB-hsdSMR-mrr)173 endA1 supE44 thi-1 recA1 gyrA96 relA1 lacHte [F′ proAB lacIqZΔM15 Tn10 (Tetr) Amy Camr]) and XL10-Gold Kan^{r} (Tetr Δ(mcrA)183 Δ(mcrCB-hsdSMR-mrr)173 endA1 supE44 thi-1 recA1 gyrA96 relA1 lac Hte [F′ proAB lacIqZΔM15 Tn10 (Tetr) Tn5 (Kanr) Amy) (Stratagene) were used for cloning purposes. BL21(DE)3 (E. coli B F⁻ dcm ompT hsdS(r_{B}⁻ m_{B}⁻) gal λ(DE3)) (Stratagene) was used for expression of fusion proteins and the target RNA.

Preparation of protein fusions containing fragments of EGFP and eIF4A was according to the method described by Vasi et al, (Vasi et al, 2004). Two sets of 5′-phosphorylated oligonucleotides were designed and purchased from Integrated DNA Technology (Coraliville, Iowa): 1. 5′-pCGAAGA TCCAGAGGA TCCCTGCTTGTCGGCCATGATATAG-3′ (SEQ ID NO.9) 2. 5′-pGGTTCTGGTAGCATGGAGCCGGAAGGCGTCATCGA-3′ (SEQ ID NO. 10), 3. 5′-pCGAAGA TCCAGAGGA TCCCTTGTACAGCTCGTCCATGCC-3′ (SEQ ID NO. 11) 4. 5′-pGGTTCTGGTAGCATTCGGATTCTTGTCAAGAAGGA-3′ (SEQ ID NO. 12). The underlined region in the oligonucleotides corresponds to the 3′ end of the A fragment (SEQ ID NO.9); the start of the F1 sequence (SEQ ID NO.10); the 3′ end of fragment B (SEQ ID NO.11); and the beginning of the F2 fragment (SEQ ID NO.12). The rest of the oligonucleotide sequence corresponds to the coding sequence for the peptide linker GSSGSS (SEQ ID NOS. 9 and 11) and GSGS (SEQ ID NOS. 10 and 12). Plasmids pMBO8 (carrying fragment A of EGFP) and pMBO9 (carrying fragment F1 of eIF4A) were cleaved with Xho1 and EcoN1 restriction enzymes; these restriction sites were located 5′ to the site where the oligonucleotides annealed. PCR was performed with these linearized plasmids and oligonucleotides 1 and 2 using the Expand Long Template FOR system (Roche Diagnostics) with the following program: 94° C. for 3 min.; 30 cycles of 94° C. for 30 s, 61° C. for 305, and 72° C. for 10 min, ending with a final 11-min elongation step at 72° C. The PCR mix was then treated with the enzyme Dpn1 to remove methylated template DNA. The PCR product of approximately 5-5 kbp was gel purified and ligated using 1 U of T4 DNA ligase (New England Biolabs). The ligated product was transformed into XL-10 competent cells (Stratagene). New chimeric plasmids carrying A-F1 (pMB12) were isolated and confirmed by sequencing. A similar protocol using oligonucleotides #3 and #4 was followed to create the chimeric B-F2 gene in vector pETDuet-1 (pMB13).

Cloning of two chimeric proteins into two MOSs of pACYCDuet-1. In order to maintain similar levels of two chimeric gene expression, the B-F2 fragment was next integrated into the second MCS of pMB12 already carrying A-F11, according to the described protocol (Geiser et al, 2001). Briefly, B-F2 was PCR-amplified from the plasmid pMB13 with flanking 5′ and 3′ vector sequences. The flanking sequences corresponded to 20 bp of DNA immediately upstream and downstream from the point of insertion in pMB12. A PCR reaction was performed with the recipient vector and the B-F2 fragment which would anneal to the uncut vector via the 20 bp flanking homology. The PCR program consisted of a denaturation step at 95° C. for 30 s followed by 18 cycles of 30 s at 95° C., 30 s at 55° C. and 8-10 mins at 68° C. using Pfu turbo DNA polymerase. The amplified product was treated with the enzyme Dpn1 for 3 hours to remove the original methylated template DNA. XL-10 competent cells were transformed with an aliquot of the purified product. Plasmids carrying both protein fragments (A-F1+B-F2) were isolated and confirmed by sequencing (pMB33). The construct A-F1+B-F2 was also generated as a negative control (pMB54). Finally, full-length EGFP was cloned into vector pACYC to serve as a positive control for EGFP expression (pMB38).

Cloning for binary aptamer-peptide chimerias. EGFP was split between amino acid residues 158 and 159 into two non-fluorescent fragments, termed α-EGFP and β-EGFP, respectively. HTLV-1 Rex peptide was fused to the C-terminus of the α-EGFP via a 10-aa flexible polypeptide linker (Gly-Ser-Ser-Gly-Ser-Ser-Gly-Ser-Gly-Ser); bacteriophage λN peptide was fused to the N-terminus of the β-EGFP (FIG. 18) via the same 10-aa linker. Multi-step PCR was performed to create a DNA fragment that encodes two fusion proteins plus T7 promoter in between. The DNA construct was inserted into the pACYCDuet-1 vector (Novagen) between the restriction sites NcoI and AvrII, which placed the insert after the first T7 promoter region and before the T7 terminator region (FIG. 17). Thus, two protein chimeras were expressed from one pACYCDuet-1 plasmid to ensure expressing equimolar amounts of both fusions. E. coli strain XL10-Gold Kan^{r} (Tetr Δ(mcrA)183 Δ(mcrCB-hsdSMR-mmr)173 endA1 supE44 thi-1 recA1 gyrA96 relA1 lac Hte [F′ proAB lacIqZΔM15 Tn10 (Tetr) Tn5 (Kanr) Amy) (Stratagene) was used for cloning. All constructs were verified by sequencing.

Aptamer cloning. A DNA sequence was designed which encodes two RNA aptamers, one of which binds to % N peptide and the other to HTLV-1 Rex peptide. The aptamers were separated by 10 thymines and restriction sites for XbaI and AvrII were added at the ends. Corresponding DNA template was custom synthesized, PCR-amplified and inserted between the XbaI and AvrII restriction sites in the pETDuet-1 vector (Novagen) under control of the T7 promoter. pETDuet-1 and pACYCDuet-1 vectors are compatible in co-expression. As a negative control, DNA sequence encoding two identical RNA aptamer sequences was synthesized similarly. E. coli strain XL10-Gold was used for this cloning. All constructs were verified by sequencing.

Growth conditions and induction. BL21(DE)₃ cells were co-transformed with pMB33 (expressing the protein chimeras) and pMB23 (expressing the target RNA). Two plasmids encoding protein and RNA components of EGFP complementation complexes were co-expressed in E. coli BL21 (DE) 3 (B F⁻ dcm ompT hsdS (r_{B}⁻ m_{B}⁻) gal λ (DE3)) (Stratagene). As a negative control, the cells were transformed with a plasmid containing two identical aptamers. As another negative control, the cells were transformed with a pETDuet-1 plasmid containing no aptamer insert. Single colony of transformed cells were grown first at 37° C. in LB media supplemented with antibiotics for 3-4 hours. Following incubation at 37° C., the cultures were diluted 300-fold into fresh medium containing the inducer isopropyl-β-D-thiogalactopyranoside (IPTG; 1 mM, or 0.01 mM for binary aptamer-peptide interactions), and grown overnight at room temperature. The optical density of the cultures was between 0.4 and 0.6 (OD₆₀₀=0.4 to 0.6) at the time of examination. The proteins were expressed in BL21(DE3)pLys competent cells (Stratagene, CA). Induction was done with 0.35 mM IPTG and cells were allowed to grow for 4 hrs at 37° C. Cells were harvested, washed and broken by sonication (3 times, 30 sec each) in a buffer containing 50 mM Tris-HCl pH 8.0, 25% sucrose, 1 mM EDTA, 10 mM DTT and 0.1% sodium azide. The inclusion bodies were washed 1 time with the same buffer and 3 times with the buffer containing 50 mM Tris-HCl pH 8.5, 100 mM NaCl, 0.5% triton X100, 1 mM EDTA, 1 mM DTT, 0.1% sodium azide and then dissolved in the buffer containing 8 M urea, 25 mM MES pH 8.5, 10 mM EDTA and 0.1 mM DTT. Proteins were refolded by droplet dilution into the same buffer containing no urea and applied to a chitin affinity chromatography resin (New England Biolabs, MA) equilibrated with the buffer (50 mM tris-HCl, pH 8.5, 0.15 M NaCl, 1 mM EDTA, 0.1% Triton X-100, 1 mM PMSF). The column was washed extensively with the same buffer. Then the column was equilibrated in a buffer containing 50 mM tris-HCl pH 7.0, 150 mM NaCl, 1 mM EDTA, 1 mM DTT, Triton X-100 0.1%, 1 mM PMSF) and left at 40° C. for 24-48 hrs for the intein cleavage to take place. Eluates were collected and protein concentration and purity were analyzed by SDS-PAGE and Coomasie Plus Protein staining (Pierce, Ill.) (see FIGS. 15 and 16). All proteins were purified in a similar way except for one occasion: for isolation of alpha-subunit of EGFP in chitin chromatography step Tris-HCl buffer was substituted by PBS buffer.

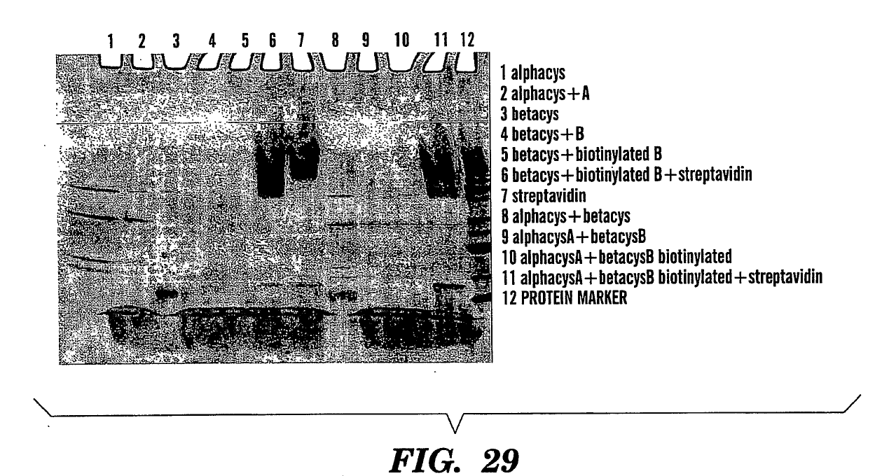

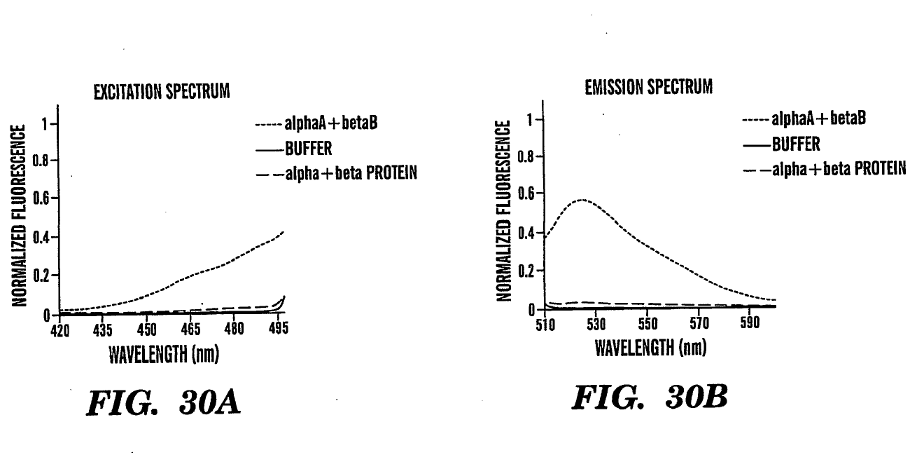

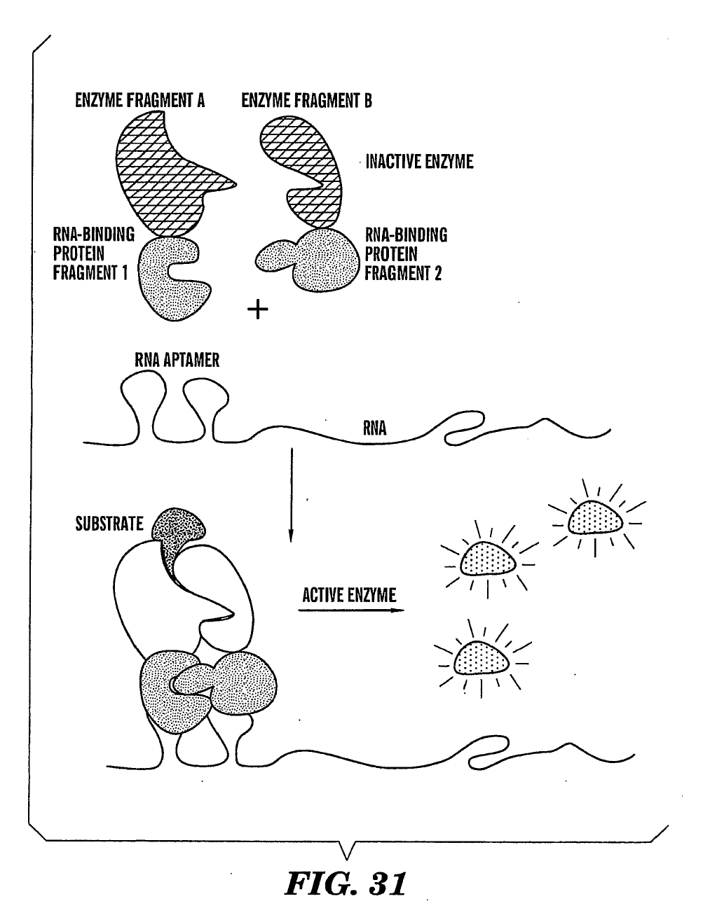

In some examples (Examples 1 and 2) proteins were refolded by droplet dilution into the same buffer containing no urea and applied to a chitin affinity chromatography resin (New England Biolabs, MA) equilibrated with the buffer (50 mM tris-HCl, pH 8.5, 0.15 M NaCl, 1 mM EDTA, 0.1% Triton X-100, 1 mM PMSF). The column was washed extensively with the same buffer. Then the column was equilibrated in a buffer containing 50 mM tris-HCl pH 7.0, 150 mM NaCl, 1 mM EDTA, 1 mM DTT, Triton X-100 0.1%, 1 mM PMSF) and left at 40° C. for 24-48 hrs for the intein cleavage to take place. Eluates were collected and protein concentration and purity were analyzed by SDS-PAGE and Coomasie Plus Protein staining (Pierce, Ill.) (see FIGS. 15 and 16). All proteins were purified in a similar way except for one occasion: for isolation of alpha-subunit of EGFP in chitin chromatography step Tris-HCl buffer was substituted by PBS buffer. In these examples, two 20-nt long complementary oligonucleotides with SH-groups at the 5′ and 3′ ends were purchased from IDT DNA Technologies. They were de-protected from the cap by incubation with a buffer containing 10 mM DTT, desalted by gel filtration through the G25 column and coupled with equimolar amounts of protein fragments in the presence of catalysts, 3 mM Cu sulfate and 9 mM 1,10-phenanthroline for 30 min. at 370° C. in the dark (Lee et al., 1994). The efficiency of coupling was close to 100% (see FIG. 29). The coupled chimeras containing two halves of EGFP and complementary oligonucleotides were mixed in equimolar amounts in a dialysis tube and dialyzed against a buffer containing 50 mM tris-HCl, pH 8.5, 0.5 M NaCl, 5 mM MgCl2 and 20 μM PEG (6000 MW). Fluorescence spectra were taken in 4 hours using Hitachi fluorescent spectrophotometer F-2500 (FIG. 30).

Cells will be grown in LB medium with appropriate antibiotic (chloramphenicol and/or streptomycin) and induced with 0.2 mM IPTG in the presence of chromogenic substrate nitrocefin. The negative controls will express no aptamer sequence, corrupted aptamer sequence and protein fusions without one of the necessary components for binding to the aptamer. The absorbance at 490 nm of the cell-free supernatant will be measured using NanoDrop ND-1000 spectrophotometer

Flow cytometry. Fluorescence measurements were obtained with a Becton-Dickinson FACSCalibur flow cytometer with a 488-nm argon excitation laser and a 515-545 nm emission filter (FL1). Cells were washed once with 1×PBS prior to assaying. Measurements were taken until 100,000 cells had been collected.

Fluorescence microscopy, imaging and data analysis. Bacterial cells in culture were immobilized between a cover slip and a thin slab of 0.8% agarose in 1×PBS. Microscopy was performed at room temperature with a Nikon Eclipse 80i inverted microscope equipped with an epifluorescence system X-Cite 120. Images were taken with exposure times of 150-300 ms using a digital B&W camera (12 bit; 20 mHz) with 100× magnification objective controlled by IPLab v.3.7 software (Scanalytics, Inc). ND4 filter was used to reduce cell photodamage. Image processing was performed using ImageJ 1.36 B software (Wayne Rasband, NIH). Fluorescent images obtained through microscopy were read into ImageJ in JPEG format and converted into 8-bit type. The threshold level was adjusted manually for each image. The upper threshold bound was automatically set to the maximum observed value and the lower threshold boundary was empirically set so that there were no pixels in the background identified as objects. To obtain total cell fluorescence the option ‘Analyze Particles’ was selected. The output contained Area of pixels, Mean, Minimum, and Maximum Grayscale of identified objects. The total fluorescence of the cells was obtained by integrating the product of the Mean minus Minimum (grayscale/pixel) by Area (of pixels). This calculation method subtracted background. Kinetics of total fluorescence changes in a single cell was calculated in a similar way, this time by applying a rectangular segment tool to the cell of interest. For quantification of fluorescence distribution along the cell, fluorescence profiles along the long axis of the bacterial cell were measured and peak surface values calculated in Microsoft Excell. Each cell was measured 3 to 4 times, and the results were averaged. Background fluorescence around each cell was quantified similarly and subtracted from the cell fluorescence profile.

Fluorescent measurements and cell imaging: Total cell fluorescence will be measured with Becton-Dickinson fluorescence activated cell sorter (FACSCalibur, with the 488 nm excitation argon laser) and will be analyzed using Excel software. Cell imaging will be performed using a confocal microscope system with automated switching between fluorescence and differential interference contrast (DIC). The magnified specimen image ×150 will be directly transmitted onto the cooled, slow-scan CCD imaging device (C4880; Hamamatsu Photonics, Bridgewater, N.J.). The computer-controlled (MetaMorph 2.5 software; Universal Imaging Corp., West Chester, Pa.) microscope will be set up to execute an acquisition protocol taking fluorescence images at 1-μm axial steps and a single DIC image corresponding to the central fluorescence image. For EGFP monitoring, an Argon laser with the 488 nm excitation line will be used and the emission window will be set between 500-540 nm. To generate a composite image of several time points, we will use 3D reconstruction feature of the Metamorph software package (Universal Imaging Corporation). RNA velocity will be measured by taking images every 1-2 min, or less if necessary, and following fluorescent RNA spot point-by-point.

Real competitive PCR design, amplification and extension. Total RNA samples from cell culture were reverse transcribed for one hour at 42° C. with 0.5 μg of random hexanucleotides and an AMV reverse transcriptase (Promega) in 20 μL total volume. Primers and competitors were designed using Sequenom's Assay Designer software and obtained from Integrated DNA Technology (Coralville, Iowa). Amplification of cDNA was performed using PCR primers at 100 nM, competitors at varying concentrations, MgCl₂ at 2.75 mM, and 200 μM dNTP using 0.1 U HotStar Taq DNA polymerase (Qiagen) in five μL with the following PCR conditions: 95° C. hot start for 15 min, followed by 45 cycles of 95° C. for 30 seconds, 56° C. for one minute, then 72° C. for 1 minute, with a final hold of 72° C. of seven minutes. After the PCR amplification, the products were treated with 0.04 U shrimp alkaline phosphatase, SAP (Sequenom), which inactivates unused dNTPs from the amplification cycles, for 20 minutes at 37° C. followed by heat inactivation at 85° C. for five minutes. For the extension cycle, 1.2 μM final concentration of extension primer and 0.6 U of ThermoSequenase (Sequenom) were added to a total reaction of nine μL with the termination mix containing specific dideoxynucleotides and deoxynucleotides for each reaction at 50 μM for each base. The extension conditions include a 94° C. hold for two minutes with 75 cycles of the following: 94° C. for five seconds, 52° C. for five seconds, and 72° C. for five seconds.

MALDI-TOF MS and Quantitative Analysis. Prior to MALDI-TOF MS analysis, salts from the reactions were removed using SpectroCLEAN resin and 16 μL of water. ASV analysis was performed using the MassARRAY system (Sequenom) by dispensing approximately 10 nL of final product onto a 384-plate format MALDI-TOF MS SpectroCHIP using a SpectroPOINT nanodispenser (Sequenom). Mass spectrometric data were analyzed using TITAN (Elvidge et al, 2005) software set at the default values.

Media: Yeast wild-type cells will be grown in YPD (2% glucose, 1% yeast extract, 2% peptone). Cells transformed with plasmids will be grown on selective synthetic dropout media (0.67% yeast nitrogen base, 2% glucose) lacking tryptophan, uracil, or leucine. Strains to be used in this study are derivatives of YPH500 (MATα, ura3-52, lys2-801amber, ade2-101ochre trp1-Δ63, his3-Δ200, leu2-Δ1) (Johnsson & Varshavsky, 1994).

Expression of conjugated detector-nucleic acid binding protein in Yeast (Eukaryotes). Plasmids used to express chimeric proteins EGFP(A)-eIF4A(F1) and EGFP(B)-eIF4A(F2) in yeast will be derivatives of the expression vector pDB20 (Becker et al., 1991) modified to express proteins fused to an HA or myc epitope tag at their N-termini. This will allow constitutive protein expression from the strong ADH1 promoter and analysis of gene expression by Western blots. Plasmid pRS4D1 (Blake et al. 2003) will be modified to express the entire message of ASH1 (in place of EGFP) with the 64 nt-long aptamer tag placed at the 3′ end of the gene (after the fourth zip-code). Integration of this plasmid into the genome of strain YPH500 will allow the regulated expression of the tagged ASH1 gene upon addition of galactose and anhydrotetracycline to the culture medium. In this way, we will follow localization of the entire ASH1 message as opposed to a reporter transcript with ASH1 3′-localization sequences (Bertrand et al., 1998; and Breach and Bloom, 2001). This might give us new insights into the mechanisms of RNA localization.

Yeast cells will be treated with zymolase and then incubated in the presence of 2 μM CCF2/AM in DMEM for 1 hr at the concentration 3×10⁵ cells/ml. The cells will be washed with PBS buffer and visualized using fluorescent microscope Nikon Eclipse 80i with a filter set for excitation at 405 nm, and 440-450 nm emission. Cell fluorescence will be also measured with fluorescence activated cell sorter (FACSaria, with the laser with excitation at 407 nm, Becton-Dickinson).

In Vitro Gel-Shift Assay. To perform the in vitro gel-shift assay, we purchased the custom-synthesized peptides and RNA aptamers listed in Table 1. RNAs were first denatured by heating at 95° C. in buffer (50 mM pH 8.0 Tris-HCl, 50 mM KCl) for 3 minutes and then slowly cooled to room temperature to renature the RNAs. Renatured RNAs and peptides were mixed in a common buffer (50 mM Tris-HCl, pH 8.0, 50 mM KCl) at 30° C. for 15 minutes. Peptide-RNA aptamer complexes were analyzed using gel electrophoresis with 15% TBE gel. RNA aptamers and peptide-RNA aptamer complexes were stained with ethidium bromide.

The examples are put forth so as to provide those of ordinary skill in the art with a complete disclosure and description of how to make and use the present invention, and are not intended to limit the scope of what the inventors regard as their invention, nor are they intended to represent the only experiments and examples that can be performed by the invention. It will be appreciated by those skilled in the art that, in light of the present disclosure, numerous modifications and changes can be made in the particular embodiments and examples exemplified without departing from the intended scope of the invention. All such modifications are intended to be included within the scope of the appended claims, and it is understood that the methods can be applied to many systems for in vivo visualization of RNA.

### REFERENCES

The references cited herein and throughout the application are incorporated herein in their entirety by reference.
1. Comolli L R, Pelton J G, Tinoco I Jr. (1998) Mapping of a protein-RNA kissing hairpin interface: Rom and Tar-Tar*. Nucleic Acids Res. 26(20):4688-95.
2. Lee, G. F., Burrows G. G., Lebert M. R., Dutton D. P. & Hazelbauer G. L. (1994) J. Biol. Chem. 269, 29920-29927.
3. Oguro A, Ohtsu T, Svitkin Y V, Sonenberg N, Nakamura Y. (2003) RNA aptamers to initiation factor 4A helicase hinder cap-dependent translation by blocking ATP hydrolysis. RNA. 9(4):394-407.
4. Roy S, Delling U, Chen C H, Rosen C A, Sonenberg N. (1990) A bulge structure in HIV-1 TAR RNA is required for Tat binding and Tat-mediated trans-activation, Genes Dev. 4(8):1365-73.
5. Saha S, Ansari A Z, Jarrell K A, Ptashne M, Jarell K A (2003) RNA sequences that work as transcriptional activating regions. Nucleic Acids Res. 31(5):1565-70.
6. Sawata S Y, Taira K. (2003) Modified peptide selection in vitro by introduction of a protein-RNA interaction. Protein Eng. 16(12):1115-24.
7. Valegard K, Murray J B, Stonehouse N J, van den Worm S, Stockley P G, Liljas L. (1997) The three-dimensional structures of two complexes between recombinant MS2 capsids and RNA operator fragments reveal sequence-specific protein. J Mol Biol. 270(5):724-38.
8. Yamamoto R, Katahira M, Nishikawa S, Baba T, Taira K, Kumar P K (2000) A novel RNA motif that binds efficiently and specifically to the Ttat protein of HIV and inhibits the trans-activation by Tat of transcription in vitro and in vivo. Genes Cells. 5(5):371-88.
9. Le, T. T., et al. Real-time RNA profiling within a single bacterium. Proc Natl, Acad Sci USA. 102, 9160-9164 (2005).
10. Demidov, V. V. et al. Fast complementation of split fluorescent protein triggered by DNA hybridization. Proc Natl Acad Sci USA. 103, 2052-2056 (2006).
11. Elvidge, G. P., Price, T. S., Glenny, L., Ragoussis, J. Development and evaluation of real competitive PCR for high-throughput quantitative applications. Anal Biochem. 339, 231-241, (2005).
12. Vasl, J., Panter, G., Bencina, M. & Jerala, R. Preparation of chimeric genes without subcloning. Biotechniques. 37, 726, 728, 730 (2004).
13. Geiser, M., Cebe, R., Dreweiio, D. & Schmitz, R. Integration of PCR fragments at any specific site within cloning vectors without the use of restriction enzymes and DNA ligase, Biotechniques. 31, 88-90, 92 (2001).
14. Pederson, T. The molecular cytology of gene expression: fluorescent RNA as both a stain and tracer in vivo. Eur J Histochem. 48, 57-64 (2004).
15. Singer R H. RNA localization: visualization in real-time. Curr Biol. 2003, 13(17):R673-675.
16. Bratu, D. P., Cha, B. J., Mhlanga, M. M., Kamer, F. R. & Tyagi, S. Visualizing the distribution and transport of mRNAs in living cells. Proc Natl Acad Sci US A. 100, 13308-13313 (2003).
17. Bertrand, E. et al. Localization of ASH1 mRNA particles in living yeast. Mol Cell. 2, 437-445 (1998).
18. Golding, I. & Cox, E. C. RNA dynamics in live Escherichia coli cells. Proc Natl Acad Sci USA. 101, 11310-11315 (2004).
19. Golding, I, Paulsson, J., Zawilski, S. M. & Cox, E. C. Real-time kinetics of gene activity in individual bacteria. Cell. 123, 1025-1036 (2005).
20. Chubb J R, Trcek T, Shenoy S M, Singer R H. (2006) Transcriptional pulsing of a developmental gene. Curr Biol. 16, 1018-1025.
21. Shav-Tal Y, Darzacq X, Singer R H. Gene expression within a dynamic nuclear landscape. EMBO J. 2006, 25(15):3469-3479.
22. Smith C A, Chen L, Frankel A D. Using peptides as models of RNA-protein interactions. Methods Enzymology 318, 423-428, 2000.
23. Bayer T S, Booth L N, Knudsen S M, Ellington A D. Arginine-rich motifs present multiple interfaces for specific binding by RNA. RNA. 2005, 11(12):1848-1857.
24. Smith C A, Calabro V, Frankel A D. An RNA-binding chameleon Mol. Cell, 2000, 6, 1067-1076.
25. Baskerville, S., Zapp, M., and Ellington, A. D. 1999. Anti-Rex aptamers as mimics of the Rex-binding element. J. Virol. 73(6):4962-4971.
26. Ye, X., Gorin, A., Ellington, A. D., and Patel, D. J. 1996. Deep penetration of an alpha-helix into a widened RNA major groove in the HIV-1 rev peptide-RNA aptamer complex. Nat Struct Biol. 3(12):1026-1033.
27. Baron-Benhamou J, Gehring N H, Kulozik A E, Hentze M W. Using the lambda N peptide to tether proteins to RNAs. Methods Mol. Biol. 2004; 257:135-154.
28. Michnick, S. W. Protein fragment complementation strategies for biochemical network mapping. Curr Opin Biotechnol. 14, 610-617, (2003).

We claim:

1. A method for the detection of nucleic acids in real time comprising:
a. expressing a nucleic acid sequence encoding a detector construct, wherein the detector construct comprises a first polypeptide fragment conjugated to a nucleic acid binding motif, and at least one other polypeptide fragment conjugated to a nucleic acid binding motif, wherein the two polypeptide fragments combine to form a detector protein in its activated state, wherein the two fragments are in an active and conformationally correct form when compared to an active wild type protein; and
b. expressing nucleic acid sequence encoding a reporter construct, wherein the reporter construct comprises a nucleotide of interest and a nucleic acid binding sequence, wherein the nucleic acid binding sequence is recognized by two or more of the nucleic acid binding motifs in the detector construct; and wherein the binding of two or more nucleic acid binding motifs of the polypeptide fragments to the nucleic acid binding sequence reconstitutes the active detector protein in real time; and
c. means for detecting the reconstituted detector protein.

2. The method of claim 1, wherein detection is in vitro or in vivo.

3. The method of claim 1, wherein the detection is in vivo.

4. The method of claim 1, wherein the nucleic acid is RNA.

5. The method of claim 1, wherein the nucleic acid is DNA.

6. The method of claim 1, 3 or 4, wherein the nucleic acid binding motif associated with the first polypeptide fragment of the detector protein is part of a full length motif, the remaining of the motif is associated with at least one other polypeptide fragment of the detector protein.

7. The method of claim 1, 3, 4 or 6, wherein the nucleic acid binding motif associated with the first polypeptide fragment of the detector protein is a full length motif that is independent from the nucleic acid binding motif associated with at least one other polypeptide fragment of the detector protein.

8. The method of claim 7, wherein the nucleic acid binding motif comprises domains of a multi-domain nucleic acid binding molecule.

9. The method of claim 1, 3 or 4, wherein the detector protein is a fluorescent protein.

10. The method of claim 9, wherein the fluorescent protein is selected from the group comprising; green fluorescent protein (GFP); enhanced green fluorescent protein (EGFP); green fluorescent protein like proteins (GFP-like); yellow fluorescent protein (YFP); enhanced yellow fluorescent protein (EYFP); blue fluorescent protein (BFP); enhanced blue fluorescent protein (EBFP); cyan fluorescent protein (CFP); enhanced cyan fluorescent protein (ECFP); a red fluorescent protein (dsRED); and modifications and fragments thereof.

11. The method of claim 10, wherein the fluorescent protein is EGFP.

12. The method of claim 11, further comprising a cleavage product located between the first and second EGFP fragments.

13. The method of claim 11, wherein the first fragment of the EGFP is amino acid 1 to approximately amino acid 158 and wherein the second fragment of the EGFP is approximately amino acid 159 to amino acid 239.

14. The method of claim 1, wherein the detector protein is an enzyme.

15. The method of claim 13, wherein the enzyme is selected from the group comprising; beta-galactosidase, beta-lactamase, beta-glucosidase, beta-glucuronidase, chloramphenicol acetyl transferase, dihydrofolate reductase (DHFR).

16. The method of claim 14, wherein the enzyme is beta-lactamase.

17. The method of claim 15, wherein the first fragment of the beta-lactamase is approximately amino acid 24 to amino acid 197 and wherein a second fragment of the beta-lactamase is approximately amino acid 198 to amino acid 240.

18. The method of claim 1, wherein the nucleic acid binding motif is a protein.

19. The method of claim 1, 3 or 4, wherein the nucleic acid binding motif protein is fragmented into two or more fragments, wherein one fragment is conjugated to the first detector polypeptide fragment and wherein the remaining fragment(s) are conjugated to one or more complementary polypeptide fragments and wherein the fragments: are (a) in the activated conformation; (b) are not active by themselves; (c) complement to reconstitute the active detector protein in real time by binding to the nucleic acid binding sequence in the reporter construct.

20. The method of claim 1, 3 or 4, wherein the nucleic acid binding motif is a MS2 coat protein and the nucleic acid binding sequence is a RNA stem-loop, or the nucleic acid binding motif is TAR and the nucleic acid binding sequence is a Tat BIV-1 stem loop, or the nucleic acid binding motif is 3 repeats of the G3/C3 stem-loop and the nucleic acid binding sequence is a transcriptional activator, or the nucleic acid binding motif is an aptamer specific for eIF4A and the nucleic acid binding sequence is an eIF4a, or the nucleic acid binding motif is an aptamer that is specific for a nucleic acid binding sequence present in the reporter construct, or the nucleic acid binding sequence present in the reporter construct is an aptamer-tag.

21. The method of claim 1, 3 or 4, wherein the means for detecting the reporter protein comprise quantitative or qualitative means.

22. The method in claim 1, wherein the method further comprises; (a) detecting a baseline signal of the detector protein in a biological sample; (b) altering the assay conditions such that there is an alteration in the contacting the nucleic acid binding sequence of the reported construct with the nucleic acid binding motifs conjugated to the polypeptide fragments of the detector protein, (c) immediately detecting a change in the activity detector protein from the biological sample, wherein a change in signal is indicative of a change in the nucleotide of interest.

23. The method of claim 1, wherein the means is selected from the group consisting of a fluorescent microscope, confocal microscope, electron microscope, Fluorescence Activated Cell Sorter (FACS), and visual inspection.

24. The method according to claim 1 wherein the detector construct and reporter construct are expressed in a cell by transformation or transfection of the cell with the constructs.

25. The method according to claim 1, wherein the detector protein is conjugated to the nucleic acid binding motif by an in frame fusion of two nucleic acids encoding the detector protein and the nucleic acid binding motif such that a fusion product is produced.

26. The method according to claim 25, wherein the fusion product includes a protease site or a tag to aid purification.

27. The method according to claim 26, wherein the tag is a 6-His tag or a glutathione-S-transferase tag or a peptide epitope.

28. A plasmid comprising DNA sequences that encode at least one of,
(i) the detector construct of claim 1;
(ii) the reporter construct of claim 1; or
(iii) both the detector construct of (i) and the reporter construct of (ii).

29. A transgenic organism having competently integrated in its genome at least one of the DNA sequences of claim 28.

30. A transgenic organism having competently integrated in its genome a functional genomic expression of;
a. a nucleic acid sequence encoding a detector construct, wherein the detector construct comprises a first polypeptide fragment conjugated to a nucleic acid binding motif, and at least one other polypeptide fragment conjugated to a nucleic acid binding motif, wherein the two polypeptide fragments combine to form a detector protein in its activated state, wherein the two fragments are in an active and conformationally correct form when compared to an active wild type protein; and/or
b. a nucleic acid sequence encoding a reporter construct, wherein the reporter construct comprises a nucleotide of interest and a nucleic acid binding sequence, wherein the nucleic acid binding sequence is recognized by two or more of the nucleic acid binding motifs in the detector construct; and wherein the binding of two or more nucleic acid binding motifs of the polypeptide fragments to the nucleic acid binding sequence reconstitutes the active detector protein in real time.

31. The transgenic organism of claim 29 or 30, wherein the transgenic organism is selected from the group consisting of a mouse, zebrafish, C. elegans, yeast, bacteria, mammalian cell, primary cell, and secondary cell.

32. A method for the detection of diseases or disorders in an individual comprising:
a. contacting DNA or RNA from an individual with a detector construct as described in claim 1, wherein the nucleic acid binding motif is specific for a particular disease or disorder; and
b. detecting a change in the signal of the detector construct, wherein the detection of a change in signal from the detector construct is indicative of the presence of a disease or disorder.

33. The method of claim 32, wherein the disease is a pathogen.

34. The method of claim 33, wherein the pathogen is selected from a group comprising a virus; influenza, bacteria, fungus, parasite, and yeast.

35. The method of claim 32, wherein the DNA or RNA is a genetic disposition to a disease.

36. The method of claim 1 or 3, wherein the detector protein fragments are designed so that they are activated immediately when reconstituted.

37. The method of claim 1, wherein the detector polypeptide protein fragments are in an active and conformationally correct form when compared to an active wild type protein, wherein complementary detector polypeptide protein fragments reconstitute the detector protein and signal phenotype in real time in the presence of the target nucleic acid.

38. The use of nucleic acid segments encoding a detector construct and a reporter construct to detect nucleic acids in real time, wherein;
(i) the detector construct comprises fragments of a detector protein which are in an active and conformationally correct form when compared to an active wild type protein; and wherein they are conjugated to a nucleic acid binding motif, and at least one other polypeptide fragment conjugated to a nucleic acid binding motif; and
(ii) the reporter construct comprises a nucleotide of interest and a nucleic acid binding sequence, wherein the nucleic acid binding sequence is recognized by two or more of the nucleic acid binding motifs in the detector construct; and wherein the binding of two or more nucleic acid binding motifs of the polypeptide fragments to the nucleic acid binding sequence reconstitutes the active detector protein in real time.

39. The use of the nucleic acid segments of claim 38, wherein the nucleic acid detected in real time is detected in vivo.

40. A kit comprising plasmids comprising DNA sequences that encode at least one of;
(i) a detector construct of claim 1;
(ii) a reporter construct of claim 1; or
(iii) both the detector construct of (i) and the reporter construct of (ii).

41. The kit of claim 40, wherein the detector construct comprises the fluorescent protein of claim 9 or 10 or an enzyme of claim 15.

42. The kit of claim 40, wherein the reporter construct comprises an apatamer of claim 20.

43. The method of claim 1, wherein the detection is in a cell selected from a group consisting of; fibroblasts, neurons, oocytes, tumor cells, virally infected mammalian cells, epidermal cells, bacterial cells and yeast cells.

44. The method of claim 43, wherein the cell is a genetically modified cell.


SEQUENCE LISTING (14 sequences)

SEQ ID NO: 1 - RNA, 35 nt, Human immunodeficiency virus
ggcuggacucguacuucgguacuggagaaacagcc

SEQ ID NO: 2 - RNA, 32 nt, Human immunodeficiency virus
ggagcuugaucccggaaacggucgaucgcucc

SEQ ID NO: 3 - RNA, 36 nt, Human T-cell lymphotropic virus
uaggcgacgguacgcaaguacucuugcgccggccua

SEQ ID NO: 4 - protein, 16 aa, Human T-cell lymphotropic virus
MPKTRRRPRRSQRKRP

SEQ ID NO: 5 - protein, 22 aa, Bacteriophage lambda
MDAQTRRRERRAEKQAQWKAAN

SEQ ID NO: 6 - protein, 17 aa, Human immunodeficiency virus
TRQARRNRRRRWRERQR

SEQ ID NO: 7 - protein, 36 aa, Human immunodeficiency virus
CFLKKGLGISYGRKKRRQRRTAPYDSKNHQDPTPEQ

SEQ ID NO: 8 - RNA, 33 nt, Bacteriophage lambda
ggauccgggcccugaagaagggcccuuuccuuu

SEQ ID NO: 9 - DNA, 40 bp, Artificial Sequence
  Description of Artificial Sequence Synthetic Oligonucleotide
cgaagatccagaggatccctgcttgtcggccatgatatag

SEQ ID NO: 10 - DNA, 35 bp, Artificial Sequence
  Description of Artificial Sequence Synthetic Oligonucleotide
ggttctggtagcatggagccggaaggcgtcatcga

SEQ ID NO: 11 - DNA, 39 bp, Artificial Sequence
  Description of Artificial Sequence Synthetic Oligonucleotide
cgaagatccagaggatcccttgtacagctcgtccatgcc

SEQ ID NO: 12 - DNA, 35 bp, Artificial Sequence
  Description of Artificial Sequence Synthetic Oligonucleotide
ggttctggtagcattcggattcttgtcaagaagga

SEQ ID NO: 13 - protein, 10 aa, Artificial Sequence
  Description of Artificial Sequence Synthetic Peptide
GSSGSSGSGS

SEQ ID NO: 14 - protein, 10 aa, Artificial Sequence
  Description of Artificial Sequence Synthetic Peptide
SGSGSGSGSG